SEQUENCE LISTING (50 sequences)

SEQ ID NO: 1 - RNA, 4395 nt, Homo sapiens
aaaauaagcagaugaacaagauaagcguuccccugggaccugaaagacgcagaagaacaaguaggaggaacauucggaggagaagaccuagcugcagcuggguccuggcaaaguuaucggguucugcucccucugccugggugagguacuggcaaaguucuccgguucugcgcccucugcccagguuugaccaaggagcuuauguuuuuaagucauggaauaaaacauggauuacuauuguggguugaacugcaucccccaaaaagaucaguggagguccuagaaacacaggacuucagaaugugacugugucugaagacaaggucauugcagauguaauuguuaagaugaaaucauacuggagcaggcugggcccacuccaguacgaccgaugucaucauaagagaagagacacagagacuccacguggagacagaggcggagaugggagcaauguggccacaaacccaggguugccacaaccagcagaaacuaggaggaggcgaggcaggagccuccccugcgccuccagagagagccuggcccugccaauaccgugauuucggacuucuuuuaaccuccagaauugugcaagaauaaauguugcuuguuuugagccacccagcuugugguccucaguuacaacagucccaggaaacuaauacaucuaguaaauaaaaugagagcgguuucucugcaugaacugaaaugauggcgggcucugcagggacaguucgcagacaucugcaaacaaauacuguccaaaccgugguaaacagccgucuuuuauuucggcgaaagccuuugggccaguuuucguauguuguuggaagcacuaaaucucucgcuggcagagaucugcaagcagcuacaagccagcggcucaccugcagcaaaacgccuggaggcagguccaugcaaugcccacgucuugcaugucauugugcaguuaaacagcuccugaaccagagagcagcugcgcccagacagcuucccgggugguaagaacuguauuugaccuccuggcagcacaaacgacugaauaacaauuaaacaaaacaggacagucuccguguuucaaaaugcgcauuuaaaccuaaaagaaacacgguguguuuacuuucugagccuggccuucccugggugcacagugagccacuucugaaagcguaugggcacacccgcuacagaaaccagcuuacacuggcucgguugccaguccuggggcauuucagaagaauugagucagaaagguguuuucugugggugauucaaaggcuccaggucuccagaaauuuguugcaugaaauggagaauaaguaggcucgggcucggacuuggccgcgugcagcuucaggggugaagcugcuuggaggccaggggccagccccagugcuuggaugcuggggugcagauccgggaaugggaugauagccagguggaaagggggagguggcuccugggucuuggagaagagggugagaauauaaccaaagauuugucugaaagccagacaguggagagucagcuuggcugugaaucauaccuaguuugagcauaaagaugagaagugaaaacagagauggggagguaaaaaaggagggggaggugaacagagaggcuuagguccccaucgauaacaguagcuaaugacacgugaacacgggaucucuuugcuugcuuuucuuuuuuuuuuuuuuuuuaagacagggucuugcucugucaccaaagcuggagugcaguggcacaaucauggcucacugcagccucaaccucccggguuccagugauccacccaucucagccuccagaguagcugggacuacaggggugcaccaccauaccuggcuauuuuuaguauuuuuuguagagacggguuuuuuugccauguugcccaggcuggucucgaacuccuggguuuaagugaucugccugccucagccucccaaagugcugggggauuacaggugugagccacugcaucuggccaagggauaucuuuugagaucuucuugccccagagcuuaauuucagaauccagcgugauuguuaaagaagaaaauaaacgggcuuuuucuauuuuaccuggagcaugucaccaggacaauuuucauccaaagaccuaguucaauggggcuccuuccccaagcuccccacacacgcuccgggaaucgauuuuuuuuacaguugucugaagugcaagucaagcuuuugcacacacacucucucucuuucucuuuuucucugucuuaaauugccagaaauguacaauuuuccuaauggagaguuuauuucaggcucaucaagauaaaauaaauuccgcuggugugcagccggccaggcgcagggcagagcggcggcaagcggcccgcgggcagagggggcagcgaggguccaugcucagcgucugaacguuacauuggcaucuggggagggggggggcuccauguguggucacgugaacuccgagaacgaaccuggcugagcacagucgaccuugucacugucaugacccaagguguccucugugacuggacauggggcggggugcguuacguagaugacucguuaagcucuaguggcuaacucagugcguccaugaaggcugugcggagaugcggccggcagaaggcaaacauagaugacgcuggggauaucgguguaaauacggguauggccaguuggucgauacagaaggguuuguuugaaggccuucgcucacacgauuguggcugcuggcaaauccaaaaucuauaaggcaggcuggcggcuggagccucaggcaggauuucuacgaggcagaauuucuccuccuucgggaaaccugcuuccccucggaaggccuucugugggcugcgugaugugcucccacaugcucaggggcaagcuccuuuauuggcuguaggcaucggccacauccccaaaacaccugcccagcaacccugagaugacugcuuggguucgucuccagguacgcuaagccugcccacauaaaacugacugccacacacugacguucuccggugagagaugggagacucagagaggccacuuccuuugccaggguuguagagcaauggaagggcuugaguggucaggucuuugggucucauugccugugguucuaaacuagcagaguguggaaugccucggcuuccacugacccuguggccccggcagggccuccaccaucaggaccgccgagcacgcccuccugccaggagcgacguccuccuuccugagaaguuucucgugggugggagggaugggaaccggacucuugacaccucccuggugccccagccccacccucgcaaggcggaccacccagaagguucuuccggccccuuuuccaaagcccuccugccucacuuaacccagcccagguuaacugaugacugugggaagagauguuuccagcagcacacugucuuguaauccaaggggcccuggaaaaagggguuucgcagcggcacuguggucucagaguccaugcgagaggaaacacaucugucccuguauuuuccgcagacacccccccucgccucccccacguccccacaggcugucauauuguuuccaguuguguuuaugguaaacucaggccauaacacgccucugucguuguaaaagauggaaagagucuuaggagagaaauuuucaucccaggccuuucaagcggcauuuucccggugguagaggugaaugggaaacaugaagcagcugccugcaggaacguuuuucccucuguggcaaggauaaacagugaaaggcguuugcugaagauggaaagagacacugugcaaaccaauaugcacagcuccugaggccugugcugggggccguggcuugcugacgucucuccaaauacgcaagucagagaagggcugacucacaggucauggauugcaaaaaauauggcacaagccugacuucgaacuuucaaagggacaaggacgugcacgccacagccacagccccagaccaggguguaggccguggggcuccagcucccuucugggcaucacugcuguguguccuugggagagugucuccgccucucuggcaccucccuucacucuggaaaccuguccaucaggccaggggacugggguggcgugcggccuaccuccucccaccuuccgagcccaaccccaaaacuggauaagcagucagcuugcacucugaacccagaacugaaacgaggauuaagcuucgcuuugugcgugccacaaaccagacaccacauccaaccuccggcucuagggccaggaacuccaaucaccaagcauuuuuggaaaaccaaaucaaagcuagauggcuuuucuaacauguuauauuguauuaaagauugaaaaugacauugagaaguaaaaauaaauuggaaauaacugg

SEQ ID NO: 2 - RNA, 1868 nt, Homo sapiens
gacccccagcccugcucccugaaucaccaccccucgagaugacacgcagugaccaauuuccucuuaaaacucccuucuguucaaaaacccuaugauccaugcacccucugucuaauccucugcgauggggccagucugcagauccgucugggcgggcugcgguagcauucuccaccugccaacacucaguugcuggugaguaaccagcauacggacucucacuuccuagaugacgccuugcuguguugcccaggguggucucuaauuccuggccuuuagcaauucucccgccuuggccuccuaaagcacugggguuacaggcaugaaccacugugcccagccugcaugauguaaucuccaaaggcaggagagagaguggucucacaacggaagggcaccagccacugagaaguccuaaggccguguuugaguucuggcucaucuuuuuugcuaauuugcucuuaugcauaucacccaaccucgcuaagccaguuucaucaucagcgagaacagcacacugacaugcagggccagcucaggcgcuaccacaagcuacacauccguccauucguuuaaucuucacagcagcccuggggucacccaggcauggaaugcacagcucugccagacagaaagcgcuuuggaugccugagaaacaacagcuuguccgggcuacugaugcgaagagaggccaugguuuccuuuuucucugcucugaauggcagccuugacgucacaacagcuaggaaccacuggagggacaaauaacaaggcgcaagccagaagcgcccugcucugucgcaggggcagggccauuuucauccaguucugaugucuucuguggccgcugcgagucggcgggaguagagacaaaugacuggagcuuuaggcguaaaggcggucucucucuaggcugaucaaacagcccuuugcccagugcuaauugccuucacggaaccuggauaaacuacaguucccgaauuaaacuuguuucugaugaacuagacuucccagggaauggguguagugauuuugcugaugguucagacaucggcacuuucccugaagggcucaacuuccuguguuuaaaagcagaccuaacaggggcuggcgcauaagcaucccgggaaauacaagaagcucaugaaagcaggcagccuuugcaagagaccugacuuauuuucuaggagaaaaacauccuuuuagggauuuuauaauuuaaguuuaagcaauuaaacaugugcaucaguacccaacugcagguguuugaacaaaugaggggaaaacgcucagaggcugccaggcuuaacucaccauuaucuaaaaugaauugucuguguaagucaucacagcaguuugaaaagcaugaucuuucauguuucaaccucacaucaaaauucuaaauuauauuuagcuaugugagugucagaucauuuguuuccauuaucaaugcgauguugggcaauuugcagacaggaucugaaagcaucauccaugacuuucauggaacagaaaagagcuuucuguugccacagauuugcccuaaguucaguccucugccuuuggagcacugacagcugucaccaaacccaaaccccagucugaacccauagugcucaagguacuuugggaaagcuaauucaggaaaaccacaggcuggagggguugucuuugagguguguauguguguguguguguaugcacauuuguuggcaguguguggacugagcacaacauauagagaggcaguguucucaggacuucugugcuccuauuuggaaaaauucggcagaagaaacaacagcucugagacacucagccccuuucaucagcucuuucuuugcuuaauuaaauacacaguucccccc

SEQ ID NO: 3 - RNA, 2007 nt, Homo sapiens
gacccccagcccugcucccugaaucaccaccccucgagaugacacgcagugaccaauuuccucuuaaaacucccuucuguucaaaaacccuaugauccaugcacccucugucuaauccucugcgauggggccagucugcagauccgucugggcgggcugcgguagcauucuccaccugccaacacucaguugcuggugaguaaccagcauacggacucucacuuccuaggcagggucucacuucaucucagccuccagaguagcugaaacuacagggcaggagagagaguggucucacaacggaagggcaccagccacugagaaguccuaaggccguguuugaguucuggcucaucuuuuuugcuaauuugcucuuaugcauaucacccaaccucgcuaagccaguuucaucaucagcgagaacagcacacugacaugcagggccagcucaggcgcuaccacaagcuacacauccguccauucguuuaaucuucacagcagcccugggacccccagcccugcucccugaaucaccaccccucgagaugacacgcaggaccaauuuccucuuaaaacucccuucuguucaaaaacccuaugauccaugcacccucugucuaauccucugcgauggggccagucugcagauccgucugggcgggcugcgguagcauucuccaccugccaacacucaguugcuggugaguaaccagcauacggacucucacuuccuggucacccaggcauggaaugcacagcucugccagacagaaagcgcuuuggaugccugagaaacaacagcuuguccgggcuacugaugcgaagagaggccaugguuuccuuuuucucugcucugaauggcagccuugacgucacaacagcuaggaaccacuggagggacaaauaacaaggcgcaagccagaagcgcccugcucugucgcaggggcagggccauuuucauccaguucugaugucuucuguggccgcugcgagucggcgggaguagagacaaaugacuggagcuuuaggcguaaaggcggucucucucuaggcugaucaaacagcccuuugcccagugcuaauugccuucacggaaccuggauaaacuacaguucccgaauuaaacuuguuucugaugaacuagacuucccagggaauggguguagugauuuugcugaugguucagacaucggcacuuucccugaagggcucaacuuccuguguuuaaaagcagaccuaacaggggcuggcgcauaagcaucccgggaaauacaagaagcucaugaaagcaggcagccuuugcaagagaccugacuuauuuucuaggagaaaaacauccuuuuagggauuuuauaauuuaaguuuaagcaauuaaacaugugcaucaguacccaacugcagguguuugaacaaaugaggggaaaacgcucagaggcugccaggcuuaacucaccauuaucuaaaaugaauugucuguguaagucaucacagcaguuugaaaagcaugaucuuucauguuucaaccucacaucaaaauucuaaauuauauuuagcuaugugagugucagaucauuuguuuccauuaucaaugcgauguugggcaauuugcagacaggaucugaaagcaucauccaugacuuucauggaacagaaaagagcuuucuguugccacagauuugcccuaaguucaguccucugccuuuggagcacugacagcugucaccaaacccaaaccccagucugaacccauagugcucaagguacuuugggaaagcuaauucaggaaaaccacaggcuggagggguugucuuugagguguguauguguguguguguguaugcacauuuguuggcaguguguggacugagcacaacauauagagaggcaguguucucaggacuucugugcuccuauuuggaaaaauucggcagaagaaacaacagcucugagacacucagccccuuucaucagcucuuucuuugcuuaauuaaauacacaguucccccc

SEQ ID NO: 4 - RNA, 1636 nt, Homo sapiens
gacccccagcccugcucccugaaucaccaccccucgagaugacacgcagugaccaauuuccucuuaaaacucccuucuguucaaaaacccuaugauccaugcacccucugucuaauccucugcgauggggccagucugcagauccgucugggcgggcugcgguagcauucuccaccugccaacacucaguugcuggugaguaaccagcauacggacucucacuuccuagaugacgccuugcuguguugcccaggguggucucuaauuccuggccuuuagcaauucucccgccuuggccuccuaaagcacugggguuacaggcaugaaccacugugcccagccugcaugauguaaucuccaaaggucacccaggcauggaaugcacagcucugccagacagaaagcgcuuuggaugccugagaaacaacagcuuguccgggcuacugaugcgaagagaggccaugguuuccuuuuucucugcucugaauggcagccuugacgucacaacagcuaggaaccacuggagggacaaauaacaaggcgcaagccagaagcgcccugcucugucgcaggggcagggccauuuucauccaguucugaugucuucuguggccgcugcgagucggcgggaguagagacaaaugacuggagcuuuaggcguaaaggcggucucucucuaggcugaucaaacagcccuuugcccagugcuaauugccuucacggaaccuggauaaacuacaguucccgaauuaaacuuguuucugaugaacuagacuucccagggaauggguguagugauuuugcugaugguucagacaucggcacuuucccugaagggcucaacuuccuguguuuaaaagcagaccuaacaggggcuggcgcauaagcaucccgggaaauacaagaagcucaugaaagcaggcagccuuugcaagagaccugacuuauuuucuaggagaaaaacauccuuuuagggauuuuauaauuuaaguuuaagcaauuaaacaugugcaucaguacccaacugcagguguuugaacaaaugaggggaaaacgcucagaggcugccaggcuuaacucaccauuaucuaaaaugaauugucuguguaagucaucacagcaguuugaaaagcaugaucuuucauguuucaaccucacaucaaaauucuaaauuauauuuagcuaugugagugucagaucauuuguuuccauuaucaaugcgauguugggcaauuugcagacaggaucugaaagcaucauccaugacuuucauggaacagaaaagagcuuucuguugccacagauuugcccuaaguucaguccucugccuuuggagcacugacagcugucaccaaacccaaaccccagucugaacccauagugcucaagguacuuugggaaagcuaauucaggaaaaccacaggcuggagggguugucuuugagguguguauguguguguguguguaugcacauuuguuggcaguguguggacugagcacaacauauagagaggcaguguucucaggacuucugugcuccuauuuggaaaaauucggcagaagaaacaacagcucugagacacucagccccuuucaucagcucuuucuuugcuuaauuaaauacacaguucccccc

SEQ ID NO: 5 - RNA, 1548 nt, Homo sapiens
gacccccagcccugcucccugaaucaccaccccucgagaugacacgcagugaccaauuuccucuuaaaacucccuucuguucaaaaacccuaugauccaugcacccucugucuaauccucugcgauggggccagucugcagauccgucugggcgggcugcgguagcauucuccaccugccaacacucaguugcuggugaguaaccagcauacggacucucacuuccaggcagggucucacuucaucucagccuccagaguagcugaaacuacagggucacccaggcauggaaugcacagcucugccagacagaaagcgcuuuggaugccugagaaacaacagcuuguccgggcuacugaugcgaagagaggccaugguuuccuuuuucucugcucugaauggcagccuugacgucacaacagcuaggaaccacuggagggacaaauaacaaggcgcaagccagaagcgcccugcucugucgcaggggcagggccauuuucauccaguucugaugucuucuguggccgcugcgagucggcgggaguagagacaaaugacuggagcuuuaggcguaaaggcggucucucucuaggcugaucaaacagcccuuugcccagugcuaauugccuucacggaaccuggauaaacuacaguucccgaauuaaacuuguuucugaugaacuagacuucccagggaauggguguagugauuuugcugaugguucagacaucggcacuuucccugaagggcucaacuuccuguguuuaaaagcagaccuaacaggggcuggcgcauaagcaucccgggaaauacaagaagcucaugaaagcaggcagccuuugcaagagaccugacuuauuuucuaggagaaaaacauccuuuuagggauuuuauaauuuaaguuuaagcaauuaaacaugugcaucaguacccaacugcagguguuugaacaaaugaggggaaaacgcucagaggcugccaggcuuaacucaccauuaucuaaaaugaauugucuguguaagucaucacagcaguuugaaaagcaugaucuuucauguuucaaccucacaucaaaauucuaaauuauauuuagcuaugugagugucagaucauuuguuuccauuaucaaugcgauguugggcaauuugcagacaggaucugaaagcaucauccaugacuuucauggaacagaaaagagcuuucuguugccacagauuugcccuaaguucaguccucugccuuuggagcacugacagcugucaccaaacccaaaccccagucugaacccauagugcucaagguacuuugggaaagcuaauucaggaaaaccacaggcuggagggguugucuuugagguguguauguguguguguguguaugcacauuuguuggcaguguguggacugagcacaacauauagagaggcaguguucucaggacuucugugcuccuauuuggaaaaauucggcagaagaaacaacagcucugagacacucagccccuuucaucagcucuuucuuugcuuaauuaaauacacaguucccccc

SEQ ID NO: 6 - RNA, 1839 nt, Homo sapiens
caggagagccguccacgccugcugugggaggaagacgcuuuccugaggcaggggccaggcagcuucuguuggcgccuggaacagcuuccgcucaucgcuucuggcggcacacacugcggaggugagacugcaaaugccgcccgccccacguggugcaggcugagugcucugguguuuguccaagcuguuccaagcccaggaggucggacucucaccgucgucuuggugauggcaacgcuguguccugccccaccucguggcugcccccaccuguaccggugagaccugaggguacgggugggguggauccugccauucccaagggcuaaguccugagacccuuuugaaaccccguuugaaaaggugacaauagcguauggcuucccgcaccaagugcacacaguacacuagcgacaggcacaaaggcaacaugaaacgggcugcucugugcucgggugucgcagaagcacgggccagagagccgguggcugcccaggaccaaagggacccacaucgaccugcugggcacuggggccuagagagugaccagagcgugauuagaagaggccggaucacagggagccccaguccacugaaccacauccagaagguagaaaggaccgauuucagaaacagucuagggcacaccaggguugagaggagccacccagagugaugcagacugucccagcucucuaaacugccacccagacacaccgguccugucccuaaagaugaaugaaacucugcaaccaacugauguuuuccccacaaucggcaauaaaucgcagccagggcgcgaacaguguucacauucgcuccuauuaaugcuuccugucucuggacgcugggccggguccagauucgcugcacuacaaggcuuccugcuggaggagugaggccucuaggcugcguuccaguuacaacuggcagcuaaugccuucacacauucgcaaaucccaggcccgccggggcaggaggcggcgagguuugccuggggucagcaacugcggcacuggcggaagccccugggaggugguucuggcccccaggcggggugaguggagcgggguccgcacggccucgcaggcgcagguuguuucucuccccuggaugccuguggggguggggacggcagagccucggacacugcccggggaucacucucaggcuacuuuucccacccugccaagcugcucgagacgucaaaggagaccaggccggucagcacacaggcuucugguaagugccgcccuccggucugggagcucagcucuccccuucugacucaccgcucggcgcugacucaucgugacggcgaguggggcuccagcuccgcggcauuucauagacagcgcggucacauuccuuuccaacagucgcucuggccucgaugucuaaggcuccgugggcagccggccuguggaugaggcggcggaaaccacauccucuucaucauccucucacgaggaaggacacugacagcuucggagaaggcugcccagugucugcgggugcccuugggagauggccuugggacacggugccugugccuguguguguacaccacgugugaacgccaacugggaacacggccccuucguaggcaaacccaggccuccuggaagucacaaguugauagcuguacucgccccuuuuggaagacgcacgcccaaggcacccgugugugcacacguauccguguguggaccucuguccacacguguguguuuugaaaagccuuucucaccacauguuaggaucacaaguguucaaaaucuaggcacacuuuucagaaguuauuucuggaaaaaauguugaaucucauaacagcaauaa

SEQ ID NO: 7 - RNA, 1362 nt, Homo sapiens
caggagagccguccacgccugcugugggaggaagacgcuuuccugaggcaggggccaggcagcuucuguuggcgccuggaacagcuuccgcucaucgcuucuggcggcacacacugcggaggugagacugcaaaugccgcccgccccacguggugcaggcugagugcucugguguuuguccaagcuguuccaagcccaggaggucggacucucaccgucgucuuggugauggcaacgcuguguccugccccaccucguggcugcccccaccuguaccggugagaccugaggguacgggugggguggauccugccauucccaagggcuaaguccugagacccuuuugaaaccccguuugaaaaggugacaauagcguauggcuucccgcaccaagugcacacaguacacuagcgacaggcacaaaggcaacaugaaacgggcugcucugugcucgggugucgcagaagcacgggccagagagccgguggcugcccaggaccaaagggacccacaucgaccugcugggcacuggggccuagagagugaccagagcgugauuagaagaggccggaucacagggagccccaguccacugaaccacauccagaagguagaaagguuguuucucuccccuggaugccuguggggguggggacggcagagccucggacacugcccggggaucacucucaggcuacuuuucccacccugccaagcugcucgagacgucaaaggagaccaggccggucagcacacaggcuucugguaagugccgcccuccggucugggagcucagcucuccccuucugacucaccgcucggcgcugacucaucgugacggcgaguggggcuccagcuccgcggcauuucauagacagcgcggucacauuccuuuccaacagucgcucuggccucgaugucuaaggcuccgugggcagccggccuguggaugaggcggcggaaaccacauccucuucaucauccucucacgaggaaggacacugacagcuucggagaaggcugcccagugucugcgggugcccuugggagauggccuugggacacggugccugugccuguguguguacaccacgugugaacgccaacugggaacacggccccuucguaggcaaacccaggccuccuggaagucacaaguugauagcuguacucgccccuuuuggaagacgcacgcccaaggcacccgugugugcacacguauccguguguggaccucuguccacacguguguguuuugaaaagccuuucucaccacauguuaggaucacaaguguucaaaaucuaggcacacuuuucagaaguuauuucuggaaaaaauguugaaucucauaacagcaauaa

SEQ ID NO: 8 - RNA, 11431 nt, Homo sapiens
cgaggaggcccaggcggugaggagccccggggcucgggccuggggcagcuggacccgugcugccgucugucgucugagagugacagagggcagcuggcuucuggguuggagcacaggggagagggaggcggccuagcgggagacggacuguggccugcguguagccggcaacuguggcaggaagaugagaggaggccgucuggagcagcuccggacccaguggaagaggccgucccgcaaggaccuggggaggagcagcagagcggggaagaaacagaugaggggcugggaaggcaggaggaggucuccagggcuggaggccaaagcacugggccccuuggcagugcgggagcugacugaccuggacugugguuagaagagagguggcaugaggugacagggaaagggaguggacaggccuucucucuuuccagacauuuaaugugaaguaaagagguuggcacgagcuguguggcagucuguggacucaagggaugaguauuuaagaugggagagaacugagccugucuagaagcccguggaagaaaugaguuacaggccacaaaaagacagggagaaaccgcuaacucgacauguggaagaggccaggcugcaaaggauuguaggauccuguaggguucuacccuuaggacauucuggaaacagcaaaaccaugaagacaaacgucagcggcugccagggguuagggaggcaggaggaacaaacagguggagcauggaggauuucggggccguggaaccccucuauggggugcuguaacaguggauacaggguaguauuaguccaaacccauagaacuuauaacaccgagcuccaagccucauguaaacuauggccuuugcauaauaauuaaguauugauguauuucaaaacaacguguagugcaggauaaauauaugcauuguuauuugucaauuacauaaacagaaauguuuuuuaaaaagcaucagcguggauaagcaaagugcgaucuucucauacggugggggauccggacccacgcugucacgggguggaccugaggcaggggcucagcgggaaaagccaaacacggaaggacggacgcugugugcugccacucacaugaggcuccuacagucaucaaauccgcagagacgggaaguagaacagugggugucaggggcugggggagggguggggaguuugcuuugaauggggacagagucucaucucgggaagaugaaaggguuccggagauggauggcagugagggcugcacaccagugugagugucccuaaugccccugaacugugcauugaaaaacaguguacauuuaggacaguacauugaugugauguguuuuuugccccaauaaaaaguaugcgacuuuaaaaaauguccaauggguuccuggccacagcgaugcaagaggccaguaacggggagacugcaggcaugggggagggcugggcaggggguagagcaucagggaacuuucuguacuuucugcuccauuuucuauaaaccuuaaaguguguucuuuaaaaaaaaaaaaaaaaaaaaaaaaaaggcuaccaguuacaaaacagaaaaagcccagaggccaaugagggcacccggcauagccugcaggccaguuggccggaggggcuggaggagguugggauggggcuggccccugcugcaugcccugccugcaccccucuccaggcugcaggugggggucuccugggguuugccuguugccccuggggcugugugcuccaugcuagcaggagccagggcagccugcccacuccugcguucccccagcagggccagcuucugcugccuagcaggucuucaggacauugugagaaagaguggauggacuggacaaacgugcucggaggaguguguguguggccacaguggcucccagggagggccaugcucaacaggagcugggauggugccacgggaggcucagggccacaaaccuggagaucaaggcacagaucaaggccguggccagccccaggcagagcugggagugagccaggccgcccgacuggggucuguggccuggccucggccccagcccgucuugguuucccacacucagggacccacuguuugugugucugucucagugggcugucauuuuuaaaaggaggugaaguucacauacccagaauuagcuauuuucagugcacaauccagcguauccagcacgaucucagugcugugcgacccucaccucggucuagcucuggagcacgugcaucccccaggcggaaacccugucacugagcggucacuccccgucuccaccaccccccucccugggcggccacccaucuauuuuugucccuauggauuugccuauucuggacauuucauauaaagggaguccaucaccacgcggcguugugugcccggccucuuccaccgaauguggcguuggugaggccuguccaugcugcagccugcaucuguccaucauuccucgcauggcccaauaauguuccauuguacaggcagaccacagugggcucgucuuuuuuuuuuuuuugagacggagugucacucugucgcccaggcuagagugcaguggugcgaucucugcucacugcaagcuccgccucccggguucacgccauucuccugccucagccuccccaguagcugggacuacaggcgccugccaucgcgccuggcuaauuuuuuguauuuuuaguagagacgggguuucaccguguuagccaggauggucucgaucuccugaccucgugauccacccgccucggccucccaaagugcugggauuauaggcgugagacaccaugcccggccugggcucgucguuuucgugguuggugcaugccggggucguuuccaccuguugccucuggugagugaugcugccgggaacaugggugugcacgugucugccugagucccugcuuucacuucuggggauagaccuggggugggauugcuggucacacgguagugcuggggguaaccggccugcguggaccggccagggcauuucccauggcagcugugcugcugacgucccacagcugugugaggcuugcucacggggcucuaacugcaugcccacuccuggcccgugggucuggaguacacagagaguggccggcucucaagcgucugagcagcucccacagccugggcgccugccugguguggaggggucggggcaggugucuugcuggggacuccagcagcuuccucuggacuugucucccggcuccaggguugcugggaggcuaguucuaaaacaggggagaagccgugugcucucucugcccacugggagccccaguguaggacgacugugcccuugugggguggggggagcuggugcccacaccacgccaggcagcucccccacccuggccucaggcagagcugccauccuucuccccuugguuuaaccauuaacuggucgccugcggugcacccggagacaauggcguguuuguucagcgcaguccgcaggggccugugcagggucuggcacagucaucaaagcuuugcgauuaagacuauuagggacgggauuugugcaaacaaagggacaggugcaggggccaggccagcugcuggggccgggugcucugcugggaggccauaaaccucccugggccauggggccucccuugccugcagcccgcucacugaggcuuugcuguuugcccaggcacagaggccgggcugcacagggcaguguguccccagggcauccugcuccccccaccccuuggcuccugggcugccuggaaccugguuggcucaggagcuuuuuaucuacagcuccucugucuauguccccuuggcagguccuuuuuaacaugaguccuccuccagugaaggaaagggucaugccagggccugcaccuagggccuccguucuccagugggugucuuugcaugaaacggccugggcccuggcacuugccccaccagcgcugccugcaaccuugucccuguaccucgggccuccaccacaucccuggaccugcacgaaggagcccugagcauggaagucaggggaagggaggucucgacaugagcccagccauggccagaguacugagcccagggccagcagccucggcuggcagugcagggugaugggucugugggccgggcugcccagggaccucggcccuggggucugaggggcaccaaugccuuuugcaggccaccauuucccuuuccacuuuccaaaugugauuggacacagguggaugcaccugccugcccagcagacagcccgguucugacccguguggagcugcguggacugggcucaccugugccuucuugcaucaaccagguguggacccugacaaggugccagccacuguccuccaggagcuaccucagggacacgcagggagggacuggacagacagauggagcgcaguccuugcugcccggccaagcccuugugggcagggcagacuccuucaagaucaucaaaugaaagcaggcaaaaaccacguccuuuugugaccuaagcuuggaagcaauguugucacuucugccucauccuauugguuagaagugagccaggagguccuguccacauccaaggugggaguccccgcaggacggggccugggaggugggcccaugggcuccagaggggcuggaccacagcaagaagaacgaggccaaccuccauggguagcgcagaccggcucaggcccugcccuucccgccgggguccccugugacaggacgcaagggcacagcugcggagcagggugggcuguuagagcugggcagaggccacaguggccagagggcuggugagaguggcucuccugcugcccuagccacuggcugaagaucugccuggggaggaguggguuugggcggccgggccuaggaccccuucauagcuaccacuaauacucauucucugcuccugagaaaccuccuccaggggccucuggggucuuggagaugagacagagggaccucaggcaggacagacaagagacccuggucucggguguccacagagcgggagccaggcaggcugggcggaaguccuaggcuuggacccuguuacgggacuuucccugucagaaugggcucaggagggagugagugcgugguccucaggugugcaagugccaaggcuggagcccacccccugggaagcagggccaggguggcuggggcggacuugaucccuggagaggagcgugggcagccagggcagggaggagggcaggcaggagggagguccaggccugugggccgacagggggugaggaggcagucuggggaccugagagaaggccgaguggcuguggggugggugaccuggggcccugacucugccuuacccuuggccauggggccacauucaugcucggcccccgcccagccugggcugggaaggcaucagccuggccaugaggcaaggggccggccaugccuggggccagagggggucccccagcccuucgccuucucuccaucuccugaguagggccccugcccugcucugacaggguggucaccuugccccgcuccgugcccaguccugagcuggcagugucuccagccuuugcagcguggcccugucugccucugugugugucugggcuccucucauuuggccgcccugcccgucuacacuggugcccccccaccugcacuguccaccgggccaggcucugucucugccaccuguugggugcuugccagacuccgccauccucuccgcucccagcuugcggcuucugccuccacuggccugggccugcccugccgccgccuccuugugcccuggcugucugcugcccccucugcugcccauuggccagccgcugagcucccuaccaggaagaaaaagggccaggccaugccggaccaggacugggcacagccuccagcacuccagguggacaggcugagaagcgcggcgagcgaggcagccugggacgggugauuucuggaacauggggccagccagccuuggcgugagguguggacagcugccagcuucuucucagggcugcucacaggcagcgggggagugaccccguuuucugcuuggcaccuggcuugccaucccgcucuaccccagggcacucagaccucaaacuccucagcugccuggaacaccagggaagcccaggcuaugugagccugugcccagcaggccagggggcaccaggacaucccccucuucucugauauuccaaaccuuccugccuuugaggaaugccucccgguccuucugguuuucuaggccaugggcucacccuggucugccagcucuggagcucugagaauauuccugcaaaaugcacaucccaggccuggccgcagacugugagucacagguagggccugggaucagcauuuucauggauucugcaggcacugcugagagcagcugcccggggucaagccagggcgugggcuguccucaucaggcuccccuucucagaggccgcccuucuggccuggccugcaccuuuccccacucacugcccaccagcugcagacacacucacuccugccucagggccucugcacggugcagacauuccucccacagcuucccagggaggcugaagagcaaggaaccugccucuccucacggagcccucugcucccgccacacggcuggauuuuucuuggaugccagcguuugacaucucuaugugagaucugcuucuucccugaucgugacauucccuccaccagcaggcagccgcaugaggaccagcgccugcucaccgcugugucccuccuacgugcggggcaggcacccagcagcagguguucggugcccacugggagcgugccugggccccugcuccugcuccucagagggaacaaacaacugaccugcugauggaggcugugaacccacacggguugucugcagacguaacggggcaucuccuaugacgcuucuggagagggcaaggcuccacggcugagggcuuucuuugggugacaaccaugugaaaugccaugaccuugugccaccccguggcagaugacucucucagaagguggugcguacccuuuccccauuaugguguggacacgcagauguggggccucucccaccucuuacaacuggggcaggcugacaaccacaguagaagggacacccuggaacucccagaccacaucauaagaggccacgcagcuuccuccuggagcucucgggcagcugcccuuggagccugacugccaugcugugaggaagcccaggugccaccgaggcggcacaugcagaugugcagccaauggcccuggcagaaccugacaaagucagcaccagccaccagaccugccagcaaggaugcucugagccgacguccacuccagcugucgucugacugcccacgagacacccugagugagcacugcccagcuaagacccgucaacuccagaacugugagaggcgaugguuccacgacuuccuggcuguagucacugcuguugggcagcugugggugacccugaagggacacucugauaagggacaucugccugcaugugugaccugagucccagccucagcuuucucaugugaaaaaucaggaccacgguagacccuaccuuauaaggcagcugcaagagugaaauacaauggaaaauguacacaagauaggguggcuguucgacgcccucuugguucuucaaaucuaaggcccaugucaaguuuuaccaucauuuugccaucaugaugagaaaagaaaugagaccagccugaccaacauggagaaaccccaucucuacuaaaaauacaaaauuagcugggcaugguggcacucgccuguaaucccagcuacuggggaggcugaggcaggagaaucacucgaacucaggagguggagguugcagugagccgagaucgcaccauugcacucuggccugggcaacaagaaugaaacucugucucacaaaaaaaaaaaaaaaaaaaaaaaaagaaauggaacuuaaacaaggaaauaguguuuuccuuguuuuguuucuugcuuauugaaagcaccuugccugauggguugagggagggauuuucauaaaucacuguuguauacacacguagagagaaaauaccagcaaguaaggguuacuguccccacauuugaguccacuuccuccgggucaauuuugucuagagucauugcaccgaggcugucccucauacacucuuccuuugggccauguacagggcugggggacacccguuccccaagaggggccuccacgauugacccgggaacuuuugcuugcucugccgguaucuagaucuuaccagcaggugccagcaaaugaucucuggacggcgccccagacucacuguccuucccuagaugggcccuugaggcuugggauggaaguguagaggggucacugccaggugauacagucagguucauccguguugagcuggggccaccaugucaugaaccuggcuguuggcagcauucacaugaaaccugcuuucagagacaauaaguguaaacaaaugucuuaggaucagugcauugugucccggauaagccccuucucccaaugucccuccacaacacucacacgaggaaaggaggcugggagguggggaggcugggagguggggaggcugacaggcugggagguggggaggcugagagguagggagguggggaggcuggaagguaggggcguggggaggcugggagguagggaggugaggaggcugggagguagggaggcugggaaguagggagauggggaggcugggagguagggagguaaggaggcugggaggcagggaggcugggaaguagggagauggggaggcugggagguagggagguggggaggcugaaagguggcaggcugggaagugaggaugcugggagguggggaggcugagagguagggaggugaggaggcugggagguagggagacugagagguagggagguggggaggcugggagguaaggaggcagggaggcugggaaguagggagguggggaggcugggagguggggaggcugagagguagggagguggggaggcugggagguggggaggcugagagguagggagguggggaggcuuggagguagaaaggugggcaggcugggagguggggaggcugggaguuggggagguuggaaggcagaggggcuaggaaguggggaggcuggaagacugggcuguugggagguagggaggcuggaggcugggcaguagggagguggagaggcugggagguggggaggcugagagguggggagguggggaggcuuggagguagaaggugggcaggcugggagguggggagccugggagguggggaggcuggaaggcugggaaguagggagguuggaaggcagaggggcugggaaguggggaggcuggaaggcugggcugcugggagguagggaggcuggaggcugggagggagggagguggggaggcuggaggcugggagggagggagguggggaggauggcuggcugggaugugaggagguguggaggcugagccuuggcagaaugugggcuuuuuucagcgaaucacuagccccugaagacagcccaggucaggugcagcaccgccccagaggccaauuagugauuugucacccucugccuuggggaacaaggcugccaggaguccugcccccagccacagcauccagcuaugcucccugcccucuggcaggagguagggaaaccucugcuuucacaucuguggggaggcccccauacuuggccccugcccuucaggcccaacccacccugucauggaauccucuagaguccuugaaggggcagagucccacccccaccaggaauuccaucggugauccuaggaacggagucccggaaggccugcuccuacggcccugggggcaggaauugaagcaggcgcucccuuuaggcagcgggacaggauggugccguacagacacuccaagucccucuccgugugacuggaugucaccaccuucugacccucaggcuccggaggguccuaggagaggauccccgcccaggguuguggagaggagcgaaccggauccuggggucaggcugcguccuggcaccuggcacacaguagggcugccaucaggcauguccacccaugcuguggaccuuugcuacugccagggcaguggaagggggcaccuaggcuguagcaggggagcucucaagggcccccccucugugcaguucagccgaggccaggccugucccaucagggugaggcuucccuuggggugagggguccuccucaccuccugaucguaaaggcccagggcugggcuagggaccccgguguuaaaagggccagaaaaugccguccaggcagauagucccagggagagggcccagagcaccugccgccaggcuagggacgcacgagggggccauggugucacccagcacccagcaccccccagaagcuccuggauccccguuaaaacugggugaucauuaaguggagugcgguugugucacagcccccagaagccccacccaccacuccccagccaccccugggagcaaggcccucacccagagagguggggugucaccggccuggccagaaccucagggugaggcaggggccucgggucacugcagggggccuccaguggacccagggguggggcugaccccagccgcucccucuggagauagaguucuggauguuucuacccuugcuccgacugccagccgccccuuggguggggcccuuugauguccccgaacucacaggugggcggggccccgaggccuccccuggcccuuugaagcugggaacucuuguggcccagucccagcagacacaggcuaacaccgcguucacccuggaguccaccggcucccccagcccagccccaggaucccaggccugcucuccucaggggucccuggagggccaguucccagccugugguccaacauccucaccucccugaaccuccauagcaauagcugcugguuuggccccuggcugagcugucuccuggucucccuugucuccauuagcacccuguucuuuagguaaggaaacugaggcacaggggcuugggaaggcagaagccccacccacgggagucaucaggcagagggagggaggugggaugggagaaauaggaggccacgggcuugcaggggucuguaucccagagucccccuaaccugaggguaccugccccccaggccucccggugaccuggcggaggggccggcggccaggucccaggcuugcugugccggcaccccacagaggcacaggggcagggaccaggccuuaucagagcggccgcccgccacccgccugcagcugacagagggauggggagaccagccguaaacccgucuguuccccuaaaggagagagggaacaaaggcauaggcuaauaaaacucagaucaaagcu

SEQ ID NO: 9 - RNA, 4247 nt, Homo sapiens
cagcgaccagaacauucauuccagggcugcucugcagcugcccaggcagcuccguggguggccacucugcuaacgugggagcacacguggcuuccaucugaugagcagucucagggaagcugggggcuucagacaccaaaugucuauuucccuccuugauuugagacucuuuuggagcucuuggagacuuccagcagaaaccugagcugccggcugccaagaaucagcgguggauuucuugaccugcucuuaauaccaauugaauaaccccuuugaaaacguucacauacaugggguacaaggugguaccuuugugcaugagacuguucuggagacggcuuaucacccagcaaaucaguguuggccaucugggacucagcgacccuuucuacgaggcuauggugaagucucacucuguugcccaggcuggagugcaguggcacgaucucggcucgcugcaaccuccaccucccagguucaagcgauccuucuguuucagccucuccaguggcugguauuacaggaacacaccaccaugccuggcuaaugcuucucuuucuauaaaugaagacgugaggccguguaagcuccaagagagcagggaucuuguuugucuuggucaccgugauauuucagcugcugacccagugcuugaagcauaaagagaaacugaggcccauaaaauugaauggcucaucucagaucaaguggccagaucaaugaagagaaguguccuuagguccggcugcuggugugaaguaugcucuggaggucugacauccacucucccucagacuuucuaauacggcaggaauuccguccaugaacuuuauaauaaacuggagcuaaaucugucagaaaauggaagaaaucuggauagcauuucaggagcacuggcaaaugccuacuccuuacuuccuaaucccaaacugucacacuguggcugcacaugugcccaagcccuuguauacucaagcuagaguugccagaugaaauggaggacauggcugggugcgguggcucagcucggagcaguggcucacgccuguaaucccagcacuuugggaggccgaggugggcugauggccugaggucaggaguucaaaaccagcguggccaacaagaugugccaguucugguaaauuuccuagcaagcuugaaggaagugaaggacaacugcaaggaagggcaggauggagcaugcagcccacguauuccccagcuugguggggcagaaugcguucuuggcuccugaggaaugggcaggcaacgcacagggguccugggaagaaagggagagaauauccugggagagccauagacccuggcacccuccugugguccuucaucaggauaaauuucuaaaagaagaauuacuggccaaaggauuccucuucaguuggaaacagcagcaaaguucacaggucgugaguggauguaaaguggacggaacagguuguugccagucuauuggaguaggaugguagaugagcagucccguuggcuaacagguguggguaugccuguuuuaaaaauuagcauguuguaggggaauauuucucaaacuuuugucacguaucuaccucuuaggcauuuuuuacuuuauucaccuguacuacuauuuaauauuuuuaaaauucaacugauuuuuuuuuuugcuuaaauacaugucuuuaaaacuggaacuuuauucgccacuguaaauggaaaaccagugucauuugccauauauagaagugaacuauauaguaaauacggugaaagugaaaugguaucaucacacuugugcuagauuccauuuccuauagaagguucuggguuucuuaaaaggcuuguuaaaaagagagaugaguaaaugugagaggaauuuaagacauacuagcaccauuaagacuuucuccuuaaaguaaucuggaggauuuaaauugagaauugaaaaaaguaacuuuauuuuacauaagaaauuauguaaaagaauuauguuauuuaaugccacauccauacgucaucaagagucaacuuggugaccacuaguggcccaugucccacacuuugagaaacguguguuggggugggaaucugaagucagaaaaacuggaguaugauaaccaccucagcacuuauuagaggccuucacuugggaaugaugacccuuccuuguaggguuguagugagaaugacagauaauauaugaaaagauaaaacauggugccagaggcauuagcaggugcugcgaaggggcuguuguauuucuuguuauacuggagugguucuccacccuggaugcauauuagaaucaucugaagggcuuuuuaaaaauacagauaucaugaccucaccccuggagauccuaauuuaauuggucagggguaggguucuggcauuguuacuuguuuaaaacaaaguuccuccuccuucccagugauuuuaauuuguagccaguguuggaaaaaccauggacuuaacccuuuccuaagagcuuagaucaauucugcagauuagcuaucugccauuuugaaaagguggcauaagcucagggaaguauucacuggacaaaaggaauucuggauggcggaagcucuaggcaccaucaggcacuugcuuugugcuggaagcugcaucaaauggcuugagcccaaaguagacauaagggcuccuuauuucccuggacacugggaaaaucauuucucuuauuccaagagaacaauuuugcugaccauggaaauacuaucucugguuuccagagguacagugcuguucagugcucuucuggagguuguaguaugaagaaaugacaaagccaaaaaggcauucugaccuagggagacuaucuuuaaagcaaaauauaaauguagggcaaaaacucugcggucucaggaggauuggauccaggggaaaauguuuaaaaggcaaacguuuagaaucaauuauacagcuucagcaaugguuugcuauugucuugcugucuuuuuguuuugcuuuguguaguguucagaauaucugcaucauguagcaucuuucuauuuuuaggagucuuuaaacuccuuuauucugaggccccagaauauucucauccaccacguacuuucacguggagaugacugacuucaucuuugacaauggcaguucccuaaccuugauccagcacgggagcaggaauuauggcgaaaauggccacccaaacugcacccagugggaaaacagcauuaacucagcuuuuaaaaaagugucaacuuggaaaauccaagaccuagaucugaugcuuagggacccacaaugccaggcagcagucugggagaugucacuucuggagcuucuaaugagaugcaaaucaccuagugaacuacuggggagcugggaaacaauguccuggguaaaagcagauaaaaauggaaugaaagaauuucuuugaagauuggaacgacauuuuaagugcuugcggauaugggacaccagauaguguugguuuuacuaagcuaauugugacggggucuuugauucccugcagcaggggacccuggggcacucgucuccagcucugccucacucuuugaggagcuaguuccaguucuacugugagugugcaaggggcuggucagcuguagcaaaaccagcacuggacaugggggaucagaagaccccggcacacauaguucaaguugugacacauauugguaugugaccuuaagugaaucccuuaccccucugcugccauguuuuauguguaaaaugauaaagagagggguggacugagguaguaauuuuuaauggagaacauugguguggcaaauuacacauguucuggccugaagauucugagcugcucuccuuggagucacauucuuucccagugugugaacaucugugugcagugagagacaugccugauggggaucuguugccugaagccagugugaaggcaggaaaaggcuuguuggaauuuaaauacgaccaaucaaaauguguuuaggcuucaaaggaaaaaguucuccauaaauacaccagaauuucauucuaacaugauucauucauucaaagagcacagcugcacaaggggaagacagcugccacaugguaacgcaguccuguaaagagucccauguggugagagacgugguugggagugagcuuggaaguggauccccaacccucguccccaaacugagacuucacaugagacugcagcucuggccaacagcuucacuguaaccucacaacagaccuugagccacaagcacccagcuaag

SEQ ID NO: 10 - RNA, 1828 nt, Homo sapiens
cagcgaccagaacauucauuccagggcugcucugcagcugcccaggcagcuccguggguggccacucugcuaacgugggagcacacguggcuuccaucugaugagcagucucagggaagcugggggcuucagacaccaaaugucuauuucccuccuugauuugagacucuuuuggagcucuuggagacuuccagcagaaaccugagcugccggcugccaagaaucagcgguggauuucuugaccugcucuuaauaccaauugaauaaccccuuugaaaacguucacauacaugggguacaaggugguaccuuugugcaugagacuguucuggagacggcuuaucacccagcaaaucaguguuggccaucugggacucagcgacccuuucuacgaggcuauggugaagucucacucuguugcccaggcuggagugcaguggcacgaucucggcucgcugcaaccuccaccucccagguucaagcgauccuucuguuucagccucuccaguggcugguauuacaggaacacaccaccaugccuggcuaaugcuucucuuucuauaaaugaagacgugaggccguguaagcuccaagagagcagggaucuuguuugucuuggucaccgugauauuucagcugcugacccagugcuugaagcauaaagagaaacugaggcccauaaaauugaauggcucaucucagaucaaguggccagaucaaugaagagaaguguccuuagguccggcugcuggugugaaguaugcucuggaggucugacauccacucucccucagacuuucuaauacggcaggaauuccguccaugaacuuuauaauaaacuggagcuaaaucugucagaaaauggaagaaaucuggauagcauuucaggagcacuggcaaaugccuacuccuuacuuccuaaucccaaacugucacacuguggcugcacaugugcccaagcccuuguauacucaagcuagaguugccagaugaaauggaggacauggcugggugcgguggcucagcucggagcaguggcucacgccuguaaucccagcacuuugggaggccgaggugggcugauggccugaggucaggaguucaaaaccagcguggccaacaagaugugccaguucugguaaauuuccuagcaagcuugaaggaagugaaggacaacugcaaggaagggcaggauggagcaugcagcccacguauuccccagcuugguggggcagaaugcguucuuggcuccugaggaaugggcaggcaacgcacagggguccugggaagaaagggagagaauauccugggagagccauagacccuggcacccuccugugguccuucaucaggauaaauuucuaaaagaagaauuacuggccaaaggauuccucuucaguuggaaacagcagcaaaguucacaggucgugaguggauguaaaguggacggaacagguuguugccagucuauuggaguaggaugguagaugagcagucccguuggcuaacagcuuguuggaauuuaaauacgaccaaucaaaauguguuuaggcuucaaaggaaaaaguucuccauaaauacaccagaauuucauucuaacaugauucauucauucaaagagcacagcugcacaaggggaagacagcugccacaugguaacgcaguccuguaaagagucccauguggugagagacgugguugggagugagcuuggaaguggauccccaacccucguccccaaacugagacuucacaugagacugcagcucuggccaacagcuucacuguaaccucacaacagaccuugagccacaagcacccagcuaag

SEQ ID NO: 11 - RNA, 730 nt, Homo sapiens
cggcggcuggcacagcggagcugcucuccaagcagcgggguucccaggcugggaaguuugcauugccguuucuuugcgggcgggcggcgagcgccgacugcaugccaccugcgagaggaagaggccggaccccaagguuuuagccugugcggcaggaggaauagcucaguugcugacaaaagcaggagaacaaggaaaagagacagcugggaguuaagcagaaggacugaauucagcgugggcuguugaaucuaagauuuuggcuggcugcucgugaagaaacgccugcagaaagcuguaaaugccggcaguggcagugagugugauggacaaggcuaaagagaagacucaugaacuuucggcacaagaaaccaaaucuuuacugagugugguggaagaaugcccaggugagcacaggcacagugccaggugcugcagaggaccugagacaaagagucacugcgcccucagagaguuuagaauccagcacugaggaaaagagaggucauggggagcacaguccuccugacaucuuaaucuaucugccaacgcuacaugccgucaagccaaggaagaauuuccuucucacuguugcuucucugaccaaggaagacauaagcacaaccaaucaccagacugaugaaauuccuuccaguuucagaaaagauuaucaccaaucccuacauuauaaaauucauacuaauaaagcacaaacauugaauucuaacau

SEQ ID NO: 12 - RNA, 337 nt, Homo sapiens
cggcggcuggcacagcggagcugcucuccaagcagcgggguucccaggcugggaaguuugcauugccguuucuuugcgggcgggcggcgagcgccgacugcaugccaccugcgagaggaagaggccguccuccugacaucuuaaucuaucugccaacgcuacaugccgucaagccaaggaagaauuuccuucucacuguugcuucucugaccaaggaagacauaagcacaaccaaucaccagacugaugaaauuccuuccaguuucagaaaagauuaucaccaaucccuacauuauaaaauucauacuaauaaagcacaaacauugaauucuaacau

SEQ ID NO: 13 - RNA, 570 nt, Homo sapiens
cggcggcuggcacagcggagcugcucuccaagcagcgggguucccaggcugggaaguuugcauugccguuucuuugcgggcgggcggcgagcgccgacucaugccaccugcgagaggaagaggccggaccccaagguuuuagccugugcggcaggaggaauagcucaguugcugacaaaagcaggagaacaaggaaaagagacagcugggaguuaagcagaaggacugaauucagcgugggcuguugaaucuaagauuuuggcuggcugcucgugaagaaacgccugcagaaagcuguaaaugccggcaguggcagugagugugauggacaaggcuaaagagaagacucaugaacuuucguccuccugacaucuuaaucuaucugccaacgcuacaugccgucaagccaaggaagaauuuccuucucacuguugcuucucugaccaaggaagacauaagcacaaccaaucaccagacugaugaaauuccuuccaguuucagaaaagauuaucaccaaucccuacauuauaaaauucauacuaauaaagcacaaacauugaauucuaacau

SEQ ID NO: 14 - RNA, 818 nt, Homo sapiens
caggcuggagugcaauggcgugaucucggcucacugcaaccuccgcuucccagguuccagcgauucuccugccucagccucccaaguagcugggauuacaggugugcaccaccaugcucggaaaaauggauggacauguuuuacugccugaacgcuggugccaguuaggccaacagucauguggcaauggcuaugaauugcaagcauugcugauuguuaucugcugcuacugccuuugagcuggagcggaacagaucaacaugggccaaacaagagccucuuuggagauuucucuacugcuucaagcacacagauuccaaguuuagaagcucaagagaagguugaaaauaaaauacauuuaguuuuugucaucuaccugggauuacuucacuauuggaaaagaauaucacaagauaauauaaauaacaaauuaccauaaaccuauucucgguggauccuuaaccuguacaaaugagaccagguguuaauuugaugacugucagagcuggaagggcacuaaggcauuaucuaguccaaugaaugauggucaaacuuaauguggcacagcagaacucuuuuuucagaaagacucguauucagaugccugauaaaaacagaaauucucuagcugaagcagggguuagggacuugagguuuuauguaugugaaauguguguguguguauguauaaauguauauguggggggguguauguguauauaugugugugaguauguauauauguauggcauucugugugccaggcucugugguaagcccuggacuuagagcaacaaauaaaggaagacauggccuca

SEQ ID NO: 15 - RNA, 798 nt, Homo sapiens
caggcuggagugcaauggcgugaucucggcucacugcaaccuccgcuucccagguuccagcgauucuccugccucagccucccaaguagcugggauuacagaaaaauggauggacauguuuuacugccugaacgcuggugccaguuaggccaacagucauguggcaauggcuaugaauugcaagcauugcugauuguuaucugcugcuacugccuuugagcuggagcggaacagaucaacaugggccaaacaagagccucuuuggagauuucucuacugcuucaagcacacagauuccaaguuuagaagcucaagagaagguugaaaauaaaauacauuuaguuuuugucaucuaccugggauuacuucacuauuggaaaagaauaucacaagauaauauaaauaacaaauuaccauaaaccuauucucgguggauccuuaaccuguacaaaugagaccagguguuaauuugaugacugucagagcuggaagggcacuaaggcauuaucuaguccaaugaaugauggucaaacuuaauguggcacagcagaacucuuuuuucagaaagacucguauucagaugccugauaaaaacagaaauucucuagcugaagcagggguuagggacuugagguuuuauguaugugaaauguguguguguguauguauaaauguauauguggggggguguauguguauauaugugugugaguauguauauauguauggcauucugugugccaggcucugugguaagcccuggacuuagagcaacaaauaaaggaagacauggccuca

SEQ ID NO: 16 - DNA, 20 bp, Artificial Sequence
  Synthetically generated oligonucleotide
ctgggtggctcctctcaacc

SEQ ID NO: 17 - DNA, 20 bp, Artificial Sequence
  Synthetically generated oligonucleotide
ccaaagggacccacatcgac

SEQ ID NO: 18 - DNA, 20 bp, Artificial Sequence
  Synthetically generated oligonucleotide
gctgatcccaggccctacct

SEQ ID NO: 19 - DNA, 20 bp, Artificial Sequence
  Synthetically generated oligonucleotide
gaatgcctcccggtccttct

SEQ ID NO: 20 - DNA, 20 bp, Artificial Sequence
  Synthetically generated oligonucleotide
cccaaccacgtctctcacca

SEQ ID NO: 21 - DNA, 21 bp, Artificial Sequence
  Synthetically generated oligonucleotide
ttcaaagagcacagctgcaca

SEQ ID NO: 22 - DNA, 20 bp, Artificial Sequence
  Synthetically generated oligonucleotide
gattcaacagcccacgctga

SEQ ID NO: 23 - DNA, 20 bp, Artificial Sequence
  Synthetically generated oligonucleotide
atgccacctgcgagaggaag

SEQ ID NO: 24 - RNA, 25 nt, Artificial Sequence
  Synthetically generated oligonucleotide
uucuggaugugguucaguggacugg

SEQ ID NO: 25 - RNA, 25 nt, Artificial Sequence
  Synthetically generated oligonucleotide
auaggagcgaaugugaacacuguuc

SEQ ID NO: 26 - DNA, 21 bp, Artificial Sequence
  Synthetically generated oligonucleotide
gcaaatcagtgttggccatct

SEQ ID NO: 27 - DNA, 21 bp, Artificial Sequence
  Synthetically generated oligonucleotide
ggtgtctacatggcagcataa

SEQ ID NO: 28 - DNA, 20 bp, Artificial Sequence
  Synthetically generated oligonucleotide
gacggaggttgagatgaagc

SEQ ID NO: 29 - DNA, 20 bp, Artificial Sequence
  Synthetically generated oligonucleotide
attcggggctctgtagtcct

SEQ ID NO: 30 - DNA, 20 bp, Artificial Sequence
  Synthetically generated oligonucleotide
tcccggaggtgctctcaatc

SEQ ID NO: 31 - DNA, 20 bp, Artificial Sequence
  Synthetically generated oligonucleotide
gggctccctctctccactcc

SEQ ID NO: 32 - DNA, 20 bp, Artificial Sequence
  Synthetically generated oligonucleotide
ttgcccaggtggcctactct

SEQ ID NO: 33 - DNA, 25 bp, Artificial Sequence
  Synthetically generated oligonucleotide
ccttctcagacaaatgcctctaagt

SEQ ID NO: 34 - DNA, 25 bp, Artificial Sequence
  Synthetically generated oligonucleotide
agaaggagatcactgccctggcacc

SEQ ID NO: 35 - DNA, 25 bp, Artificial Sequence
  Synthetically generated oligonucleotide
cctgcttgctgatccacatctgctg

SEQ ID NO: 36 - DNA, 25 bp, Artificial Sequence
  Synthetically generated oligonucleotide
caagcactaccaccagcactgttac

SEQ ID NO: 37 - DNA, 25 bp, Artificial Sequence
  Synthetically generated oligonucleotide
gcaatcaggaggcacaggacataat

SEQ ID NO: 38 - RNA, 7058 nt, Homo sapiens
cagcgaccagaacauucauuccagggcugcucugcagcugcccaggcagcuccguggguggccacucugcuaacgugggagcacacguggcuuccaucugaugagcagucucagggaagcugggggcuucagacaccaaaugucuauuucccuccuugauuugagacucuuuuggagcucuuggagacuuccagcagaaaccugagcugccggcugccaagaaucagcgguggauuucuugaccugcucuuaauaccaauugaauaaccccuuugaaaacguucacauacaugggguacaaggugguaccuuugugcaugagacuguucuggagacggcuuaucacccagcaaaucaguguuggccaucugggacucagcgacccuuucuacgaggcuauggugaagucucacucuguugcccaggcuggagugcaguggcacgaucucggcucgcugcaaccuccaccucccagguucaagcgauccuucuguuucagccucuccaguggcugguauuacaggaacacaccaccaugccuggcuaaugcuucucuuucuauaaaugaagacgugaggccguguaagcuccaagagagcagggaucuuguuugucuuggucaccgugauauuucagcugcugacccagugcuugaagcauaaagagaaacugaggcccauaaaauugaauggcucaucucagaucaaguggccagaucaaugaagagaaguguccuuagguccggcugcuggugugaaguaugcucuggaggucugacauccacucucccucagacuuucuaauacggcaggaauuccguccaugaacuuuauaauaaacuggagcuaaaucugucagaaaauggaagaaaucuggauagcauuucaggagcacuggcaaaugccuacuccuuacuuccuaaucccaaacugucacacuguggcugcacaugugcccaagcccuuguauacucaagcuagaguugccagaugaaauggaggacauggcugggugcgguggcucagcucggagcaguggcucacgccuguaaucccagcacuuugggaggccgaggugggcugauggccugaggucaggaguucaaaaccagcguggccaacaagaugugccaguucugguaaauuuccuagcaagcuugaaggaagugaaggacaacugcaaggaagggcaggauggagcaugcagcccacguauuccccagcuugguggggcagaaugcguucuuggcuccugaggaaugggcaggcaacgcacagggguccugggaagaaagggagagaauauccugggagagccauagacccuggcacccuccugugguccuucaucaggauaaauuucuaaaagaagaauuacuggccaaagaaaguucacaggucgugaguggauguaaaguggacggaacagguuguugccagucuauuggaguaggaugguagaugagcagucccguuggcuaacagguguggguaugccuguuuuaaaaauuagcauguuguaggggaauauuucucaaacuuuugucacguaucuaccucuuaggcauuuuuuacuuuauucaccuguacuacuauuuaauauuuuuaaaauucaacugauuuuuuuuuuugcuuaaauacaugucuuuaaaacuggaacuuuauucgccacuguaaauggaaaaccagugucauuugccauauauagaagugaacuauauaguaaauacggugaaagugaaaugguaucaucacacuugugcuagauuccauuuccuauagaagguucuggguuucuuaaaaggcuuguuaaaaagagagaugaguaaaugugagaggaauuuaagacauacuagcaccauuaagacuuucuccuuaaaguaaucuggaggauuuaaauugagaauugaaaaaaguaacuuuauuuuacauaagaaauuauguaaaagaauuauguuauuuaaugccacauccauacgucaucaagagucaacuuggugaccacuaguggcccaugucccacacuuugagaaacguguguuggggugggaaucugaagucagaaaaacuggaguaugauaaccaccucagcacuuauuagaggccuucacuugggaaugaugacccuuccuuguaggguuguagugagaaugacagauaauauaugaaaagauaaaacauggugccagaggcauuagcaggugcugcgaaggggcuguuguauuucuuguuauacuggagugguucuccacccuggaugcauauuagaaucaucugaagggcuuuuuaaaaauacagauaucaugaccucaccccuggagauccuaauuuaauuggucagggguaggguucuggcauuguuacuuguuuaaaacaaaguuccuccuccuucccagugauuuuaauuuguagccaguguuggaaaaaccauggacuuaacccuuuccuaagagcuuagaucaauucugcagauuagcuaucugccauuuugaaaagguggcauaagcucagggaaguauucacuggacaaaaggaauucuggauggcggaagcucuaggcaccaucaggcacuugcuuugugcuggaagcugcaucaaauggcuugagcccaaaguagacauaagggcuccuuauuucccuggacacugggaaaaucauuucucuuauuccaagagaacaauuuugcugaccauggaaauacuaucucugguuuccagagguacagugcuguucagugcucuucuggagguuguaguaugaagaaaugacaaagccaaaaaggcauucugaccuagggagacuaucuuuaaagcaaaauauaaauguagggcaaaaacucugcggucucaggaggauuggauccaggggaaaauguuuaaaaggcaaacguuuagaaucaauuauacagcuucagcaaugguuugcuauugucuugcugucuuuuuguuuugcuuuguguaguguucagaauaucugcaucauguagcaucuuucuauuuuuaggagucuuuaaacuccuuuauucugaggccccagaauauucucauccaccacguacuuucacguggagaugacugacuucaucuuugacaauggcaguucccuaaccuugauccagcacgggagcaggaauuauggcgaaaauggccacccaaacugcacccagugggaaaacagcauuaacucagcuuuuaaaaaagugucaacuuggaaaauccaagaccuagaucugaugcuuagggacccacaaugccaggcagcagucugggagaugucacuucuggagcuucuaaugagaugcaaaucaccuagugaacuacuggggagcugggaaacaauguccuggguaaaagcagauaaaaauggaaugaaagaauuucuuugaagauuggaacgacauuuuaagugcuugcggauaugggacaccagauaguguugguuuuacuaagcuaauugugacggggucuuugauucccugcagcaggggacccuggggcacucgucuccagcucugccucacucuuugaggagcuaguuccaguucuacugugagugugcaaggggcuggucagcuguagcaaaaccagcacuggacaugggggaucagaagaccccggcacacauaguucaaguugugacacauauugguaugugaccuuaagugaaucccuuaccccucugcugccauguuuuauguguaaaaugauaaagagagggguggacugagguaguaauuuuuaauggagaacauugguguggcaaauuacacauguucuggccugaagauucugagcugcucuccuuggagucacauucuuucccagugugugaacaucugugugcagugagagacaugccugauggggaucuguugccugaagccagugugaaggcaggaaaaggguaagaaccauuagcccagaggcccuuuaaagucccuuccaacuuuaagaugcuguaacuccagaguaaucgccaguuaaccucuuggagccucaucugugaaacugacauaaaugcuuuugcuuuucagcuuguuggaauuuaaauacgaccaaucaaaauguguuuaggcuucaaaggaaaaaguucuccauaaauacaccagaauuucauucuaacaugauucauucauucaaagagcacagcugcacaagguagguuccccugaaccacuuucugggucauuugguuacugaugggcuaaaggacaucauuaaaaccucaccucagccuuugcccagcuguuagcaaaaccugcaguguauagauuaauauugcacagcuguacaaauccccacccagguccauaaauuauaaaauauuuuuacacauagagagggcagucauuaguaugaaugacaugauuugggggagguuauaucuacaaaugucuggcaggugugugugugugugagagagagagagagagagggagagagagauugaguggaaauagaaaagaaugucaguaguagaccaaaauaaaaucacaaauauaauuuuuuucuugcauuuaauuaauaagauguaaaaaugaaaucaaugaaguguuaguggugagaaaugcaguugugaucuuucagucuuaaucuucaaaaaagccaagaaaauaguaaguaucugagagaagcaaagauuaaauagaaagguaugguuugguuauuuuccuaauuugugccgaauuuuggaacaaauuucuuaaguacauuacauuauaaaaucaauuugguccaacuauccaguaaacauuaagacguuuguacaaguagggcagcacaaagcuuucaguucucugcucuaaucaagaauuuaaauuucaguucuucagggucacagagaacuaccaaauuucuuugccuuguuuuacaauccaauaugagauuaguaaaaauccguuacggaaauaaugcauaaaaugaugacuucucaugcccucccugaccugaggagguugcaagagaucugaauuucuccacuggacucaagauuucucaacaaauuagcaucagcagcuuccugcccccaccccuuuuaaauaucccuauacucagguggggcgugguggcucacaccuguaaucccagcacuuugggaggcugagggggguggaucacuugaggccaggaguuuaagaccagccuguccaacauagcaaaacccugucucuacuaaaaauacaaaaaauuagccgggccugguggcguaugccuguaaucccagcuacucgggagggugaggcaggagaaucgcuugaacccgggaggcagagguugcagugagccaagaucaggccacugcacuccagccuggacggcagagugauacuccaucuaaaaaaaaaaaaaaaaaaacccuacacucugagugaccuaccugaucaguauagcuauaagcaccuuccuauaugucauaaaaaacaaaccaagcuuaaccaaaaaguaaaugguuacaguuguugcuaacaaagcugagaaauggguaucuccuguguuauacaagugauauuucagccuugacaaggaaauuauugccuacacacacacacacacacacacacacacacacgcacaaaagcaaaugcuuuguccaugaaguucgaccaaacuaaaagauaguuccagugcaauguaauuuuaaguuuccaaaugaacauauuccaggaggcucauaucagugguccucagcucuugaccccugaaggaaacauucuuucagggacuugguugucuugguuuagucaaauaaaauauuaacaaucagaaaccuggcagcauuuaucugacaauauagagaaaaucauauuaacaugagcucuucacuggaaugaucuuggacauccaugauggcaccgggaaacgcgccaccagcucuggaagggugggaggcucuggcugacuuggcaccuucccacuuccagcucucguggccgcucauccugcaugagauggggacccagcagagagggguuaaauggguugagaagccaguccagccaggggucauuagcaaucaaggaccagcauuaaaaggcagcaggagacuaagguucucugacaggcuuggcuccaggcaccaucuggaaggaaagaggcugucaaaguuggcuuaaaaaaaaaaaaaaagaaauucauggagaucuuuucuuuucaauaacauaacacaaacaguuauguuauuuccagcguggcuccuuccuuggaaguuagguuaucuucaaagugcucuguuuuugagaguagggccaggcagucacauuucuaggccuguuucuggccaugaugaaccaguuauuggcaccuggguugggaccagcccaagaccaaguguauggccaacaaacucuuauacuuugauaguggcgagagaucguaacacacaugcuucauaauaagguaacuggaauaugucagaucuaaaguauggggcacagcucauauccaauaaugcauuaaucauauuccacacacuauauaauacgaucugugacuaaaaagaaaaaaacagacagcuuugcuuagaaaaccuguguucaagcugaucaucuaaguugugauuuuugacacaacuuaaauaauucuaaaacaagccuggguucacuuucuaagguaauuuguacaaggaaaugucagucagggguguugcauauuacauacaugugguuacgaacuugguuuacauuauugauuaaauucauuuucucuuucucuuuuuuagaccuuuggauaucuccuccuccuuccccuuaucuauaaauauguaagaaagaaaacauguuuaaaauacaauauuuuauuucuuuugaucacagauuagacuuaaagaacagagaugcccuauaaugugaucuuuaagagauauuacaaagcuuccaaucucacugugaggaucguuaauauauacauauauuuuauuauacuuuaaguucuaggguacauguacacaaugugcagguuuguuacauauguauacauguguuguguugguuugcugcacccauuaacuugucauuuacauuagguauaucuccuaaugcuaucccucccccgcucccccca

SEQ ID NO: 39 - RNA, 8139 nt, Homo sapiens
cagcgaccagaacauucauuccagggcugcucugcagcugcccaggcagcuccguggguggccacucugcuaacgugggagcacacguggcuuccaucugaugagcagucucagggaagcugggggcuucagacaccaaaugucuauuucccuccuugauuugagacucuuuuggagcucuuggagacuuccagcagaaaccugagcugccggcugccaagaaucagcgguggauuucuugaccugcucuuaauaccaauugaauaaccccuuugaaaacguucacauacaugggguacaaggugguaccuuugugcaugagacuguucuggagacggcuuaucacccagcaaaucaguguuggccaucugggacucagcgacccuuucuacgaggcuauggugaagucucacucuguugcccaggcuggagugcaguggcacgaucucggcucgcugcaaccuccaccucccagguucaagcgauccuucuguuucagccucuccaguggcugguauuacaggaacacaccaccaugccuggcuaaugcuucucuuucuauaaaugaagacgugaggccguguaagcuccaagagagcagggaucuuguuugucuuggucaccgugauauuucagcugcugacccagugcuugaagcauaaagagaaacugaggcccauaaaauugaauggcucaucucagaucaaguggccagaucaaugaagagaaguguccuuagguccggcugcuggugugaaguaugcucuggaggucugacauccacucucccucagacuuucuaauacggcaggaauuccguccaugaacuuuauaauaaacuggagcuaaaucugucagaaaauggaagaaaucuggauagcauuucaggagcacuggcaaaugccuacuccuuacuuccuaaucccaaacugucacacuguggcugcacaugugcccaagcccuuguauacucaagcuagaguugccagaugaaauggaggacauggcugggugcgguggcucagcucggagcaguggcucacgccuguaaucccagcacuuugggaggccgaggugggcugauggccugaggucaggaguucaaaaccagcguggccaacaagaugugccaguucugguaaauuuccuagcaagcuugaaggaagugaaggacaacugcaaggaagggcaggauggagcaugcagcccacguauuccccagcuugguggggcagaaugcguucuuggcuccugaggaaugggcaggcaacgcacagggguccugggaagaaagggagagaauauccugggagagccauagacccuggcacccuccugugguccuucaucaggauaaauuucuaaaagaagaauuacuggccaaagaaaguucacaggucgugaguggauguaaaguggacggaacagguuguugccagucuauuggaguaggaugguagaugagcagucccguuggcuaacagguguggguaugccuguuuuaaaaauuagcauguuguaggggaauauuucucaaacuuuugucacguaucuaccucuuaggcauuuuuuacuuuauucaccuguacuacuauuuaauauuuuuaaaauucaacugauuuuuuuuuuugcuuaaauacaugucuuuaaaacuggaacuuuauucgccacuguaaauggaaaaccagugucauuugccauauauagaagugaacuauauaguaaauacggugaaagugaaaugguaucaucacacuugugcuagauuccauuuccuauagaagguucuggguuucuuaaaaggcuuguuaaaaagagagaugaguaaaugugagaggaauuuaagacauacuagcaccauuaagacuuucuccuuaaaguaaucuggaggauuuaaauugagaauugaaaaaaguaacuuuauuuuacauaagaaauuauguaaaagaauuauguuauuuaaugccacauccauacgucaucaagagucaacuuggugaccacuaguggcccaugucccacacuuugagaaacguguguuggggugggaaucugaagucagaaaaacuggaguaugauaaccaccucagcacuuauuagaggccuucacuugggaaugaugacccuuccuuguaggguuguagugagaaugacagauaauauaugaaaagauaaaacauggugccagaggcauuagcaggugcugcgaaggggcuguuguauuucuuguuauacuggagugguucuccacccuggaugcauauuagaaucaucugaagggcuuuuuaaaaauacagauaucaugaccucaccccuggagauccuaauuuaauuggucagggguaggguucuggcauuguuacuuguuuaaaacaaaguuccuccuccuucccagugauuuuaauuuguagccaguguuggaaaaaccauggacuuaacccuuuccuaagagcuuagaucaauucugcagauuagcuaucugccauuuugaaaagguggcauaagcucagggaaguauucacuggacaaaaggaauucuggauggcggaagcucuaggcaccaucaggcacuugcuuugugcuggaagcugcaucaaauggcuugagcccaaaguagacauaagggcuccuuauuucccuggacacugggaaaaucauuucucuuauuccaagagaacaauuuugcugaccauggaaauacuaucucugguuuccagagguacagugcuguucagugcucuucuggagguuguaguaugaagaaaugacaaagccaaaaaggcauucugaccuagggagacuaucuuuaaagcaaaauauaaauguagggcaaaaacucugcggucucaggaggauuggauccaggggaaaauguuuaaaaggcaaacguuuagaaucaauuauacagcuucagcaaugguuugcuauugucuugcugucuuuuuguuuugcuuuguguaguguucagaauaucugcaucauguagcaucuuucuauuuuuaggagucuuuaaacuccuuuauucugaggccccagaauauucucauccaccacguacuuucacguggagaugacugacuucaucuuugacaauggcaguucccuaaccuugauccagcacgggagcaggaauuauggcgaaaauggccacccaaacugcacccagugggaaaacagcauuaacucagcuuuuaaaaaagugucaacuuggaaaauccaagaccuagaucugaugcuuagggacccacaaugccaggcagcagucugggagaugucacuucuggagcuucuaaugagaugcaaaucaccuagugaacuacuggggagcugggaaacaauguccuggguaaaagcagauaaaaauggaaugaaagaauuucuuugaagauuggaacgacauuuuaagugcuugcggauaugggacaccagauaguguugguuuuacuaagcuaauugugacggggucuuugauucccugcagcaggggacccuggggcacucgucuccagcucugccucacucuuugaggagcuaguuccaguucuacugugagugugcaaggggcuggucagcuguagcaaaaccagcacuggacaugggggaucagaagaccccggcacacauaguucaaguugugacacauauugguaugugaccuuaagugaaucccuuaccccucugcugccauguuuuauguguaaaaugauaaagagagggguggacugagguaguaauuuuuaauggagaacauugguguggcaaauuacacauguucuggccugaagauucugagcugcucuccuuggagucacauucuuucccagugugugaacaucugugugcagugagagacaugccugauggggaucuguugccugaagccagugugaaggcaggaaaaggcuuguuggaauuuaaauacgaccaaucaaaauguguuuaggcuucaaaggaaaaaguucuccauaaauacaccagaauuucauucuaacaugauucauucauucaaagagcacagcugcacaaggggaagacagcugccacaugguaacgcaguccuguaaagagucccauguggugagagacgugguugggagugagcuuggaaguggauccccaacccucguccccaaacugagacuucacaugagacugcagcucuggccaacagcuucacuguaaccucacaacagaccuugagccacaagcacccagcuaagcugcagggagacccccugaaacuauugcuacggaauaaaagaugaaaugcuccugauuauuguaaauacaaaguugcaugcaggauuguguaaagacaaugccagguuggacugccagaaugagccaacugcacgugaugugauucccccugcagagagccuaugaauggacgugcagucagggagguuucacaucaccaagauuccuauccuagaaaggcagauguucauagcucuaggaauggaaugcgacccuuguggagagccuauaaacggaugcauggggguggggcgccuguccauauggauaagauagggcuagaaacgcccucaucuugccacagcucuucuaggccucuuuaggguuaaggcauacucccuucugagaauuucuggucuaacugguugucuagcuucacauccuguuuccguggauuguuuguaaccagcuuuuguugcaauuguuacugcugauuaauaucuugcuaaucauagguuauggaaagauugugcuucuguuuuaaggcucuguuagaaauuacugacgcacacacuauauuguaaauucuuaucucucuaugcuguacuucuacauacaaauguacuguacuucuacauacaaauguacuguacuucuacauacaaauguuauguuaauuacuucauccccaugugaccaucucaccucauaaucaaaugacccuaaaucccucacuaaccaacccccacccucacuuaacuuaauaauaaacgcugguauauccagagcauuguuggcaccaugggaccagaaggcggugaccccccuggacccagcuuucacuaucuugugugugucuauuauuucccaaccugcugaucugccugggaacaaagagagagccccauugcauugugggcugcugaccagauccugaagaugcaugcaacaacgagacagagaaauuugcugaaaaagcaccacagcaggaaacaacuaacacauccaaucaucaaaaagaagaaaagaccacgcuaacuccuuuacaacagacaauccagacagccauccugaacaacuugucuccagcgauccaggucuggcucaaccucugccuccuccugaugacacugauccuucuaccucuguuacgccacagacuguuaaaaacuauacauauugggccuauauuccuuuuccuccucuuauucgagccgugacauggauggaugcuccuaucgaggucuauguuaaugauaguauuuggaugccugguucucuagaugaucguuguccugcacaaccuucagaagaaggaaccccuuucaauaucacuuuagguuuuagguauccaccuuucugccugggacccacuaauguaugucucucauuagauauucaaacuugggcugucacacuaccgucuggucacucugucccuccuuuaggacacuugauaucagggcucucauuaaaaccucuaaggcagaucaaaacaguaaucacugauuauauucacacaucccaauauaagccuuuagaccuguguguccucucaacuugucuucaaaugcugacaaaauuaauauggaaagacuguguuagcucagaaggaaccguguuauuuaauucuucucacuacaccauuguugauugggcuccuaaaggucauauuacuaaugauugcucucaaggucacagagauugucaacauuuucucuauaauauuacuuaucaaaaacguagugacagcccuccccuauuauaucguagauuuaacuccuuuuuuccucuuaaguggaaaggggcagggguugcgccuccaaagccaaggcucauuguuccccacauaggaccugaacauuccaaauuauggagauuaacaauagcuaugacugguaugagaguuugggcuggagaaaguguuauaaguaaauccacuuugucaccucaaaauguauauacucucuauauggaguccaacaaaacuauaccacuuaaaaguuguguuaaaccaccauauauguuauuagcaggaaaguugcauauuaguucaaaaaccaacauaauuaccuguguuaauuguuacuuguauacuuguauugacucauccuuuaaucaauaucauaguauuuuaauagucagagccagacaagauauuuggcucccuguagccuuacauaggccuugggaaucuucccccuuuauccauguuauuaacaauauucuauagaaaauucuuaaaaggaguaaacaauuuauuuuuacauuaauugcaauaauaaugggcuugauugcuguuacugugauugcugcuacugcuggaguugcauuacguccaucuauucaaacuguucauuuuguggauaaauggcaaaaaaauucuacucggauguggaauucucagguauugaucaaaaauuggcuaaucaaauuaaugaucuaagacaaacuguuauauggaugggagguagaauuaugaguuuagaacauagauuacaaaugcaaugugauuggaauacuucugauuuuuguauaacuccauuucaauauaaugagucuguucacaauugggaaucaguaaaaugccauuuacaaggaagugaagauaauuuaaagcuuuagacauaagcaaacuaaaagaacagauuuuugaggccucucaagcacacuuaacugcuuuacccggugcugaaguuuuagacgguaucucugagggauuaucuaaucucaaccccauucaauagguaaaaucuuugggaggauccacuaucguuaauuuuguucuguguauaauuuguacuauugguuuauuguucauguguaaaauuggaaaaaauauucuucaguccaauugugaucagugccaagcuaugauugcuaugguucauuuaaaucagagagaagggggagauguaaggagacccccugaaacuguugcuauggaauaaaagaugaaaugcuccugauuauuguaaauacaaaguugcaugcaggauuguguaaagacaaugccagguuggacugccagaaugagccaacugcacgugaugugauucccccugcagagagccuaugaauggacgugcagucagggagguuucacaucaccaagauuccuauccuagaaaggcagauguucauagcucuaggaauggaaugcgacccuuguggagagccuauaaacggaugcauggggagguggcuguccguauggauaagauagggcuauaaacgcccucaucuugccacagcucuucuaggccucuuuaggguuaaggcauacucccuucugggaauuucuggucuaaccgguugucuagcuucacguccuguuuccauggauuguuuguaaccagcuuuuguugcaauuguuacugcugauuaauaucuugcuaaucauagguuauggaaagauuguguuucuguuuuaaggcucuguuagaaauuacugacacacacacuauauuguaaauucuuaucucucuauacuguacuucuacauacaaauguacuguacuucuacauacaaauguuaugugaaagaauuacuucauccccaugugaccaucucaccucauaaucaaaugacccuaaaucccucacuaaccuacccccacccucacuaaacuuaauaauaaacgcugguauauccagugcauuguuggcaccaugggaccggaaggcagugaacccccuggacccagcuuucacuaucuugugugugucuauuauuucucaaccugccaauacaccugggaacaaagagagagccccauugcauugugggcugcugaccagauccugcaauacuaaucugcguccagauuccugacccacagaaacugugauauaacaaauguuugccguuuuaagccacuaaguuuugguguaauuuguaagaagcaauagauaaugaauacauuccuccuccucccc

SEQ ID NO: 40 - RNA, 26993 nt, Homo sapiens
cagcgaccagaacauucauuccagggcugcucugcagcugcccaggcagcuccguggguggccacucugcuaacgugggagcacacguggcuuccaucugaugagcagucucagggaagcugggggcuucagacaccaaaugucuauuucccuccuugauuugagacucuuuuggagcucuuggagacuuccagcagaaaccugagcugccggcugccaagaaucagcgguggauuucuugaccugcucuuaauaccaauugaauaaccccuuugaaaacguucacauacaugggguacaaggugguaccuuugugcaugagacuguucuggagacggcuuaucacccagcaaaucaguguuggccaucugggacucagcgacccuuucuacgaggcuauggugaagucucacucuguugcccaggcuggagugcaguggcacgaucucggcucgcugcaaccuccaccucccagguucaagcgauccuucuguuucagccucuccaguggcugguauuacaggaacacaccaccaugccuggcuaaugcuucucuuucuauaaaugaagacgugaggccguguaagcuccaagagagcagggaucuuguuugucuuggucaccgugauauuucagcugcugacccagugcuugaagcauaaagagaaacugaggcccauaaaauugaauggcucaucucagaucaaguggccagaucaaugaagagaaguguccuuagguccggcugcuggugugaaguaugcucuggaggucugacauccacucucccucagacuuucuaauacggcaggaauuccguccaugaacuuuauaauaaacuggagcuaaaucugucagaaaauggaagaaaucuggauagcauuucaggagcacuggcaaaugccuacuccuuacuuccuaaucccaaacugucacacuguggcugcacaugugcccaagcccuuguauacucaagcuagaguugccagaugaaauggaggacauggcugggugcgguggcucagcucggagcaguggcucacgccuguaaucccagcacuuugggaggccgaggugggcugauggccugaggucaggaguucaaaaccagcguggccaacaagaugugccaguucugguaaauuuccuagcaagcuugaaggaagugaaggacaacugcaaggaagggcaggauggagcaugcagcccacguauuccccagcuugguggggcagaaugcguucuuggcuccugaggaaugggcaggcaacgcacagggguccugggaagaaagggagagaauauccugggagagccauagacccuggcacccuccugugguccuucaucaggauaaauuucuaaaagaagaauuacuggccaaagaaaguucacaggucgugaguggauguaaaguggacggaacagguuguugccagucuauuggaguaggaugguagaugagcagucccguuggcuaacagguguggguaugccuguuuuaaaaauuagcauguuguaggggaauauuucucaaacuuuugucacguaucuaccucuuaggcauuuuuuacuuuauucaccuguacuacuauuuaauauuuuuaaaauucaacugauuuuuuuuuuugcuuaaauacaugucuuuaaaacuggaacuuuauucgccacuguaaauggaaaaccagugucauuugccauauauagaagugaacuauauaguaaauacggugaaagugaaaugguaucaucacacuugugcuagauuccauuuccuauagaagguucuggguuucuuaaaaggcuuguuaaaaagagagaugaguaaaugugagaggaauuuaagacauacuagcaccauuaagacuuucuccuuaaaguaaucuggaggauuuaaauugagaauugaaaaaaguaacuuuauuuuacauaagaaauuauguaaaagaauuauguuauuuaaugccacauccauacgucaucaagagucaacuuggugaccacuaguggcccaugucccacacuuugagaaacguguguuggggugggaaucugaagucagaaaaacuggaguaugauaaccaccucagcacuuauuagaggccuucacuugggaaugaugacccuuccuuguaggguuguagugagaaugacagauaauauaugaaaagauaaaacauggugccagaggcauuagcaggugcugcgaaggggcuguuguauuucuuguuauacuggagugguucuccacccuggaugcauauuagaaucaucugaagggcuuuuuaaaaauacagauaucaugaccucaccccuggagauccuaauuuaauuggucagggguaggguucuggcauuguuacuuguuuaaaacaaaguuccuccuccuucccagugauuuuaauuuguagccaguguuggaaaaaccauggacuuaacccuuuccuaagagcuuagaucaauucugcagauuagcuaucugccauuuugaaaagguggcauaagcucagggaaguauucacuggacaaaaggaauucuggauggcggaagcucuaggcaccaucaggcacuugcuuugugcuggaagcugcaucaaauggcuugagcccaaaguagacauaagggcuccuuauuucccuggacacugggaaaaucauuucucuuauuccaagagaacaauuuugcugaccauggaaauacuaucucugguuuccagagguacagugcuguucagugcucuucuggagguuguaguaugaagaaaugacaaagccaaaaaggcauucugaccuagggagacuaucuuuaaagcaaaauauaaauguagggcaaaaacucugcggucucaggaggauuggauccaggggaaaauguuuaaaaggcaaacguuuagaaucaauuauacagcuucagcaaugguuugcuauugucuugcugucuuuuuguuuugcuuuguguaguguucagaauaucugcaucauguagcaucuuucuauuuuuaggagucuuuaaacuccuuuauucugaggccccagaauauucucauccaccacguacuuucacguggagaugacugacuucaucuuugacaauggcaguucccuaaccuugauccagcacgggagcaggaauuauggcgaaaauggccacccaaacugcacccagugggaaaacagcauuaacucagcuuuuaaaaaagugucaacuuggaaaauccaagaccuagaucugaugcuuagggacccacaaugccaggcagcagucugggagaugucacuucuggagcuucuaaugagaugcaaaucaccuagugaacuacuggggagcugggaaacaauguccuggguaaaagcagauaaaaauggaaugaaagaauuucuuugaagauuggaacgacauuuuaagugcuugcggauaugggacaccagauaguguugguuuuacuaagcuaauugugacggggucuuugauucccugcagcaggggacccuggggcacucgucuccagcucugccucacucuuugaggagcuaguuccaguucuacugugagugugcaaggggcuggucagcuguagcaaaaccagcacuggacaugggggaucagaagaccccggcacacauaguucaaguugugacacauauugguaugugaccuuaagugaaucccuuaccccucugcugccauguuuuauguguaaaaugauaaagagagggguggacugagguaguaauuuuuaauggagaacauugguguggcaaauuacacauguucuggccugaagauucugagcugcucuccuuggagucacauucuuucccagugugugaacaucugugugcagugagagacaugccugauggggaucuguugccugaagccagugugaaggcaggaaaaggguaagaaccauuagcccagaggcccuuuaaagucccuuccaacuuuaagaugcuguaacuccagaguaaucgccaguuaaccucuuggagccucaucugugaaacugacauaaaugcuuuugcuuuucagcuuguuggaauuuaaauacgaccaaucaaaauguguuuaggcuucaaaggaaaaaguucuccauaaauacaccagaauuucauucuaacaugauucauucauucaaagagcacagcugcacaagguagguuccccugaaccacuuucugggucauuugguuacugaugggcuaaaggacaucauuaaaaccucaccucagccuuugcccagcuguuagcaaaaccugcaguguauagauuaauauugcacagcuguacaaauccccacccagguccauaaauuauaaaauauuuuuacacauagagagggcagucauuaguaugaaugacaugauuugggggagguuauaucuacaaaugucuggcaggugugugugugugugagagagagagagagagagggagagagagauugaguggaaauagaaaagaaugucaguaguagaccaaaauaaaaucacaaauauaauuuuuuucuugcauuuaauuaauaagauguaaaaaugaaaucaaugaaguguuaguggugagaaaugcaguugugaucuuucagucuuaaucuucaaaaaagccaagaaaauaguaaguaucugagagaagcaaagauuaaauagaaagguaugguuugguuauuuuccuaauuugugccgaauuuuggaacaaauuucuuaaguacauuacauuauaaaaucaauuugguccaacuauccaguaaacauuaagacguuuguacaaguagggcagcacaaagcuuucaguucucugcucuaaucaagaauuuaaauuucaguucuucagggucacagagaacuaccaaauuucuuugccuuguuuuacaauccaauaugagauuaguaaaaauccguuacggaaauaaugcauaaaaugaugacuucucaugcccucccugaccugaggagguugcaagagaucugaauuucuccacuggacucaagauuucucaacaaauuagcaucagcagcuuccugcccccaccccuuuuaaauaucccuauacucagguggggcgugguggcucacaccuguaaucccagcacuuugggaggcugagggggguggaucacuugaggccaggaguuuaagaccagccuguccaacauagcaaaacccugucucuacuaaaaauacaaaaaauuagccgggccugguggcguaugccuguaaucccagcuacucgggagggugaggcaggagaaucgcuugaacccgggaggcagagguugcagugagccaagaucaggccacugcacuccagccuggacggcagagugauacuccaucuaaaaaaaaaaaaaaaaaaacccuacacucugagugaccuaccugaucaguauagcuauaagcaccuuccuauaugucauaaaaaacaaaccaagcuuaaccaaaaaguaaaugguuacaguuguugcuaacaaagcugagaaauggguaucuccuguguuauacaagugauauuucagccuugacaaggaaauuauugccuacacacacacacacacacacacacacacacacgcacaaaagcaaaugcuuuguccaugaaguucgaccaaacuaaaagauaguuccagugcaauguaauuuuaaguuuccaaaugaacauauuccaggaggcucauaucagugguccucagcucuugaccccugaaggaaacauucuuucagggacuugguugucuugguuuagucaaauaaaauauuaacaaucagaaaccuggcagcauuuaucugacaauauagagaaaaucauauuaacaugagcucuucacuggaaugaucuuggacauccaugauggcaccgggaaacgcgccaccagcucuggaagggugggaggcucuggcugacuuggcaccuucccacuuccagcucucguggccgcucauccugcaugagauggggacccagcagagagggguuaaauggguugagaagccaguccagccaggggucauuagcaaucaaggaccagcauuaaaaggcagcaggagacuaagguucucugacaggcuuggcuccaggcaccaucuggaaggaaagaggcugucaaaguuggcuuaaaaaaaaaaaaaaagaaauucauggagaucuuuucuuuucaauaacauaacacaaacaguuauguuauuuccagcguggcuccuuccuuggaaguuagguuaucuucaaagugcucuguuuuugagaguagggccaggcagucacauuucuaggccuguuucuggccaugaugaaccaguuauuggcaccuggguugggaccagcccaagaccaaguguauggccaacaaacucuuauacuuugauaguggcgagagaucguaacacacaugcuucauaauaagguaacuggaauaugucagaucuaaaguauggggcacagcucauauccaauaaugcauuaaucauauuccacacacuauauaauacgaucugugacuaaaaagaaaaaaacagacagcuuugcuuagaaaaccuguguucaagcugaucaucuaaguugugauuuuugacacaacuuaaauaauucuaaaacaagccuggguucacuuucuaagguaauuuguacaaggaaaugucagucagggguguugcauauuacauacaugugguuacgaacuugguuuacauuauugauuaaauucauuuucucuuucucuuuuuuagaccuuuggauaucuccuccuccuuccccuuaucuauaaauauguaagaaagaaaacauguuuaaaauacaauauuuuauuucuuuugaucacagauuagacuuaaagaacagagaugcccuauaaugugaucuuuaagagauauuacaaagcuuccaaucucacugugaggaucguuaauauauacauauauuuuauuauacuuuaaguucuaggguacauguacacaaugugcagguuuguuacauauguauacauguguuguguugguuugcugcacccauuaacuugucauuuacauuagguauaucuccuaaugcuaucccucccccgcuccccccaccccacgacaggccccuggugugugauguuccucuuacuguguccgaguguacucacuguucaauucccaccuaugagugagaacacgcaguguuugguuuucuguccuugagauaguuugcucagaaugaugguuuccagcuucauccaugucacuacaaaggacaugaacccaucauuuuuuauggcugcauaguauuccaugguguauaugugccacauuuucuuaauccagucuaucauuguuggacauuuggauugguugcaagucuuugauacugugaauaaugccacaauaaacauacaugugcaugugucuuuauaguagcaugauuuauaauccuuuugguauauacccaguaaugggaucccugggucaaaugguauuucuaguucuggauccuugaggaaucgccacacugucuuccacaaugguugaacuaguuugcacucccaccaacaguguaaaaguguuccuauuucuccacauccucuccagcaccuguuguuuccuuuuuaauuaucgccauucuaacuggugugagaugguaucucauugugguuuugauuugcauuucacugaugaccagugaugaucagcauuuuuucaugugucuguuggcugcauaaaugucuucuuuugaaaagugucuguucauauccuuugcccacuuuuugaugggguugauuuuuucuuguaaauuuguuugaguucuuuguagauucuggauuucagcccuuugucagauggguagauugcaaaaauuuccucccauucuguagguugccuguucacucugaugguaguuucuuuugcugugcagaagcucuuuaguuuaauuagaucccauuugucaauuuuggcuuuuguugccauugcuuuugcuguuuuagucaugaaguccuugcccaugccuauguccugaaugguauugccuagguuuucuucuacaguuuuuaugguuuuaggucuaacauguaagucuuuaauccaucuugaauuaauuuuuguauaagguguaaggaagggauccaguuuccgcuuucuacauauggcuagccaguuuucccagcaccauuuauuaaauagggaauccuuuccccauugcuuguuuuuuguuagguuuaucaaagaucagaugguuguagaugugugguguuauuucugaggccucuguucuguuccauuggucuauaucucuguuuugguaccaguaccaugcuguuuugguuacuguagccuuguaguauuguuugaaguccgguagcgugauucuuccagcuuuguuuuuuuggcuuaggauugucuuggcaaugugggcucuuuuuuguuuccauaugaacuuuaaaguaguuuuuuuccaauucugugaagaaagucauugguagcuugauggggauggcauugcaucuauaaauuaccuagggcaguauggccauuuucacaauauugauucuuccuauccaugagcauggaauguucuuccauuuguuuguguccucuuuuauuucguugaucagugguuuguaguucuccuugaagagguccuucacaacccuuguaaguugcauuccgagguauuuuauucucuuuguaggaauugugaaugggaguucacucaugauuuggcucucuguuugucuguuacugguguauaagaaugccugugauuuuugcacauugauuuuguauccugagacuuugcugaaguuccuuauuagcuuaaggagauuuugggcugagacaaugggguuuucuaaauauacaaucaucugcaaacagggacaauuugacuuccuuuuuuccuaacugaauaccguuucuuucuuucucuugccugauugcccuggccagaacuuccaacacuauguuggauaggaguggugagagagggcaucccugucuugugccaguuuucaaagggaaugcuuccaguuuuugcccauucaguaugauacuggcuguggguuuaucauaaauagcucuuacuauuuugagauauguuccuugaauaccuaguuuauugagaguuuuuagcaugaagcgcuguugaauuauauccaaggccuuuucugcaucuguugagauaaucaugugguuuucuucuuugguucuguuuauaugauggauuaaguuuauugauuugcguauguugaaccagccuugcauccagggaugaauccaaauugauuuugguggauaagcuuuuugaugugcugcuggauuugguuugccaguauuuuauugaggauuucugcaucgauguucgucagggauauuggucuaaaauucucuuguuuuguugugucucugccaggcuuugguaucaggaugaugcuggccucauaaaaugaguuagggaggauucccucuuuuucuauugauuggaauaguuucaaaaggaaugguaccagcuccuccuuguaccucugguagaauucggcugugaaucugucugacccuggacuuuuuuugguugguaggcuguuaguuauugccucaauuucagagccuguuauuggucuauucagcaauucagcugucuuuggaggguauaagugucgaggaauuuauccauuucuucuagauuuucuaguuuauuugcauagagguguuuauaguauucucugaugguaguuuguauuucugugggaucaguggugauaugccguuuaucauuuuuuauuaugucuauuugauucuucucucuuuucuucuuuauuagucuugcuagcagucuaucaaucuuauugaucuuuucaaaaaaccagcuccuggauucguugguuuuuuugaaggguuuuuugugucucuaucucuuucaguucuccucugaucuuaguuauuucuuggcuuccgcuagcuuuugaauguguuugcucuugccucucuaguucuuuuaauugugauguuagggugucaauuuuagaucuuuccugcuuucucuugugggcauuuagugcuauaaguaaguuucccucuacacacugcuuuaaaugugucccagagauucugguauguugugucuuuguucucguugguuucaaagaacaucuuuauuucugccuucauuucguuauguacccaguagucauucaggagcagguuguucaguuuccauguaguugagcgguuuugagugaguuucuuaauccugaguucuaauuugacuguggucugagagacaguuuguuaugauuucuguuguuuuacauuugcugaggagugcuuuacuuccaacuauguggucaauuuuggaacaagugcgauguggugcugaaaagaauguauauucuguugauuugggguggagaguucuguagaugucuauuaggucugcuuggugcagagcugaguucaaguccuggauauccguguuaaucuucugucucguugaucuaauauugacagugggguguuaaagucucccacuauuauugugugggagucuaagucuccuugugggucucucagacuugcuuuaugaaucugggugcuccuguauauauuuaggauaguuagcucuucuuguuggaaucaucucuuuaccguuauguaaugguuuuccuugucucuuuugaucuuuguugguuuaaacucuguuuuaucagagaguagcauugcaaccccugccuuuuuuuguuuuccguuugcuugguagaucuuccuccaucccuuuauuuugagccugugugugucucugcacauugaauguugaauauuggcccccacucucuucuggcuuguagaguuucugcugagagauccacuguuagucugaugggcuucccuuuguggguaacccgaccuuucucucuggcugcccuuaacauuuuuuccuucauuucaacuuugguguaucugacaauuaugugucuuggaguugcucuucuccaggaguaucuaucuuugugguguucucuguauuuccugaauuugaauguuggccugccuugcuagguuggggaaguucuccuggguaauauccugaagaggguuuuccagcuugguuccauucuccccaucacuuucagguacaccaaucagauguagauuuggucuuuucacauaguccccuauuucuuggaggcuuuguucauuucuuuuuauucuuuuuucucuaaacuucucuucucacuucauuucauucauuugaucuuccaucauugauacccuuucuuccaguugauagagucggcuacugaagcuugugcauucaucacguaguucucuugccaugguuuucagcuccaucagguccuuuaaggacuucucugcauugguuauucuaguuagccauucaucuaaucuuuuuucaagguuuuuaacuucuuugcgauggguucgaacuuccuccuuuagcucggagaaguuuggucaucugaagccuucuucucucaacucgucaaagucauucuccguccagcuuuguucuguugcuggcaaggagcugcguuccuuuggaggagaaggggcacucugguuuuuagaauuuucagcuuuucuacucugguaucuucccaucuuugugguuuuaucuaccuuuggucuuugaugauggugaccuacagaugggguuuugguguggauguccuuuuuguugauauugaugcuacuccuuuguguuuguuaguuuuccuucuaacagucaggucccucaguugcaggucuguuggaguuuguuggagguccacuccagacccuguuugccuggguauaagcagcggaggcugcagaacagcaaauguugcugccugauccuuccucugaagcuucgucucagaggggcacccagcuguaugaggugucagucggccucuacugggaggugucuccuaguuaggcuacacgggggucagggacccacuugaggaggcaggcuaucuguucucagagcucaaacacugugcugggagaaccacugcucucuucagagcugucacacagggacguuuaaaucugcagaaauuucugugccuuuuguuuagcuaugcccugccuccagagguggagucuacagaggcaggcaggccucguugagcugcggugggccccacucaguucgagcuuccuggcagcuuuguuuaccuacucaagccucagcaaugguggacaccccuccaccagccaggcuugccgccuugcaauuugaucucggacuagcaaugagcaaggauccaugggggugggacccacugcgccaagugcaggauauaaucuccuggugugcugcaugcuaagaccuucagaaaagugccacguuuagguggagugucccgauuuuccugguacagucugucacgguuucccuuggcuaggaaagggaaaucccccgaucccuugugcuucucaggugaggcaaugccccgcccugcuucagcucgcccuccaugggcugcacccacuuucugaccagucucaaugagaugaagcagguaccucagcuggaaaugcagaaaucacccaucuucuguguugaucaugcugggagguguagaauggagcuguuccuauuuggccgucuuggaauggaaaucagaucauuaauauuauagcugaguucauuagaggaauuacauucuaguucauuauuguguggguuuuguuuguuuguuuuuagauagagucucgcucuguugcgcaggcuggagugcaguggcacaaucuuggcuaacugcaaugucugccuccaagguucaagugauucuccugucucagccucgugaguagcugggauuauaggcaugugccaccaugcccagcuaauuuuuuguauuuuaguagagauggaguuucaccauguugcccaggcuggucuugaacuccuaagcucaggcaauccaccugccuuggccucccaaaguguuaggauuacaggugugagccaccgcacccagcccguuguguggcuuuuaacauuagucuucaguauacccuuaguucugauauaaaaaagguauuacaagaaaaaggauucaggguauauuuguuuuuagauggugaugguacuuuuaugauaauuuuacccucugcuaaaaucacccucaugaagcuucuggccaguuuauagaugcuugggaugggggaaaaauaacagggaagaucuaagacucuguagccucagauacagccucaccagagggugagucaggugauacuggcugcuaauccaggaagugggcaucauuuggagggguaaucaggguggcuggcaguccagcagaaguccagcagaaaugcaagauucuggggauugaaucuggaucauuuuaaaucauccaauccuaaacuuuugacaguuuaugcccauacuggccagccugucugagacaguguauuaaucggagguccaauuacacagaacagaguuuguuuaaaaacagacaagcaagucccagagacagaacacuguggguauggaugaauccuguccuuaacguucuucgguugggcucacucugauuuugacuuuucucacuguucucauggagacuuuugguuuucauaggauguaagugugccuacauaaacaugacacuugagucaugaaaacccagaccugaucagaguuauaggguuccugggaaagacuaugggaagauucuuuuaaaaaaaauuguuugguaaaaauauccaaacuugaagccuacuauccaauccuauacuaaagcuucacaaaagaugcuguauggaccauuguugguaaagugaacuucuaggggggcuuugaugccuucaguggcuguagguuuuguaauaucagaguuugauuuauaagaauauaagaggacaguuccaagauggccgaauaggcacagcuccagucuacagcucccagcgugaacgaugcagaagacgggugauuucugcauuuccaacugagguaccggguucaucucacaggggcuugucggacggugggugcagcgcacugagcgugagccaaagcagggcaaggcauugccucauccaggaagcacaaggggucagggaauucccuuucacagccaagcaaagcugugacagauggcaccuggaaaaucgggucacucccacccuaauacugugcuuuuccaacggucuuagcaaacagcacaccaggagauuauauccugcgccuggcucagagggucccaugcccacggagccucgcucauugcuagcacagcagucugagaucgaacugcaaggcagcagugaggcuggggguggggcgccugccacugcugaggcuugaguagguaaacaaagcagccaggaagcucgaacuggguggagcccaaugcagcucaaggaggccugccugccucuguagacuccaucucuggaggcaggucauagccaaacaaaaggcagcagaaaccucugaagacuuaaaugucccugucugacagcuuugaagagaguagugguucucccagcacagaguuugagaucugagaacagacagacugccucaagugggucccugacccccgaguagccuaacugggaggcaccccccaguaggggcagacugacaugucacauggcgagguaccccucugagacgaaaccuccagaaaaacgaucagacagcaauauuugcuguucagcaauauucgcugaucuacacccuccgcuggugauacccaggcaaacagggucuggaguggaccuccaacaaacuccaacagaccugcagcugaagguccugacuguuagaaggaaaacuaacaaacagaaaggacauccacaccaaaaccccaucuguaggucaccaucaucaaagaccaaagguagauaaaccacaaagauggggaaaaaacagaacagaaaaacagaaaauucuaaaaaucagagcgccucuccucuuccaaaggaacacagcuccucaccagcaacagaacaaagcuggacggagaaugacuuugacgaauugagagaagaaggcuucagaugaccaaacuucuccgagcuaaaggaggaaguucgaacccaucgcaaagaaguuaaaaaccuugaaaaaagauuagaugaauggcuaacuagaauaaccaaugcagagaaguccuuaaaggaccugauggagcugaaaaccauggcaagagaacuacgugaugaaugcacaagcuucaguagccgauucuaucaacuggaagaaaggguaucaaugauggaagaucaaaugaaugaaaugaagugagaagagaaguuuagagaaaaaagaauaaaaagaaacgaacaaagccuccaagaaauaggggacuaugugaaaagaccaaaucuacaucugauugguguaccugaagugauggggagaauggaaccaagcuggaaaacccucuucaggauauuacccaggagaacuuccccaaccuagcaaggcaggccaacauucaaauucaggaaauacagagaacaccacaaagauagauacuccuggagaagagcaacuccaagacacauaauugucagauacaccaaaguugaaaugaaggaaaaaauguuaagggcagccagagagaaaggucggguuacccacaaagggaagcccaucagacuaacaguggaucucucagcagaaacucuacaagccagaagagagugggggcccauauucaacauuccuaaagaaaugaauuuucaacccagaauuucauauccagccaaacuaagcuucauaagugaaggagaaauaaaauccuuuacagacaagcaaaugcugagagauucugucaccaccaggccugcccuacaagagcuccugaaggaugcacuaaacauggaaaggaacaacugguaccagccacugcaaaaacuugccaaauuguaaagaccaucuauccucggaagaaacugcaucaacuaaugagcaaaauagccagcuaacaucauaaugacaggaucaaauucacacauaacaauauuaaccuuaaauguaaaugggcuaaaugcuccaauugacacagacuggcaaauuggaugaaaagucaagacccaucagugugcuguauucaggaaacccaucucacgugcagagacacacauaggcucaaaauaaagggauggagacagaucuaccgagcaaauggaaaacaaaaggcagggguugcaauccuagucucugauaaaacagacuuuaaaccaacaaagaucaaaagagauaaagaaggccauuacauaaucguaaagggaucaauucaacaagaagagcuaacuauccuaaauauauaugcacccaguacaggagcacccagauucauaaagcaaguccuuagagaccuacaaagagacuuagacucccacacaauaauaaugggagacuucaacaccccacggucaacauuaaacagaucaacgagacagaaaguuaacaaggauauccaggaaaugaauuuagcucugcuccaagcagacauaauagacaucuacagaacuuuccaccccaaaucaacagaauauacauucuucucagcaccacaccaaaccuauuccaaaauugaccacauaguuggaaguaaagcacuccucagcagagguaaaagaacagaaauuauaacaaacugucucucagaccacagugcaaucaaacuagaacucaggauuaagaaacucacucaaaaccgcucaacuacgugggaacugaacaaccugcuccugaaugacuacuggguacauaacgaaaugaaggcagaaauaaagauguucuuugaaaccaaugagaacaaagacacaacauaccagaaucucugggacacauuuaaagcaguguguagagggaaacuuauagcacuaaaugcccacaagagaaagcaggaaagaucuaaaauugacacccuaacaucacaauuaaaagaacuagcgaggcaagagcaaacacauucaaaagcuagcagaaagcaagaaauaacuaagaucagagcagaaaugagggagauagagacacaaaaaacccuuugaaaaaucaaugaauccaggagcugguuuuuugaaaagaucaacaaaauugauagacugcuagcaagacuaaugaagaaaagagagaagaaucaaauagaugugauaaagggcauaucaccacggaucccacagaaauacaaacuaccaucagagaauacuauaaacaccucuaugcaaauaaacuagaaaaucuagaagaaauggauaaauaccuccacacauauacccucccaagacuaaaccaggaagaagcugaaucucugaauagaccaauaacaggcucugaaauugaggcaauaauuaacugcuuaccaaccaaaaaaaguccaggaccagacggauucacagccaaauucuaccagagguacaaggaggagcugguaccauuccuucugaaacuauuccaaucaauggaaaaagacgaaauccucccuaauucauuuuaugaggccagcaucauccugauaccaaagccuggcagagacacaacaaaacaagagaauuuuagaccaauaucccugaugaacaucgaugcaaaaauccucaauaaaauacuggcaaaccgaauccagcagcacaucaaaaagcuuauccaccaugaucaagugggcuucauuccugggaugcaaggcugguucaacauaugaaaaucaauaaacuuaauccaucauauaaacagaaccaaagagaaaaaccacaugauuaucucaauagaugcagaaaaggccuuugacaaaauucaacagccuuuuaugcuaaaaacucucaauaaauuagguauugaugagauguauaucaaaauaauaagagcuauuuaugacaaacccacagccaauaucauacugaaugggcaaaaacuggaagcauucccucugaaagcuggcacaagacaaggaugcccucucucaccacuccuauccaacauaguguuggaaguucuggccagggcaaucaggcaggagaaagaaagaaaggguacucaauuaggaaaagaggaagucaaaucgucccuguuugcagaugacaugauuguauaucuagaaagucccaucgucucagcccaaaaucuccuuaugcugauaagcaaccucagcaaagucucaggauacaaaaucaacgugcaaaaaucacaagcagucuuauacaccaauaacagacagacaaaucaugagugaacucccauucacaauuccuacaaagagaauaaaaugccucggaaugcaacuuaaaagggaugugaaggaucucuucaaggagaacuacaaaccacugcucuacgaaauaaaagaggacacaaacaaauggaagaacauuccaugcucauggauaggaagaaucaauauugugaaaaugaccauacugcccuagguaauuuauagaugcaaugccauccccaucaagcuaccaaugacuuucuucacagaauuggaaaaaacuacuuuaaaguucauauggaaccaaaaaagagcccacauugccaagucaaucugaagccaaaagaacaaagcuggaggcaucacacuaccugacuucaaacuauacuacgaggcuacaguaaccaaaauagcaugguacugguaccaaaacagaaauauagaccaauggaacagaacagaggccucagaaauaauaccacacaucuacacuaccugaucuuugacaaaccugacaaaaacaagaaaugggaaaaggauucccuauuuaacaaauggugcugggaaaacuggcuagccauauguagaaagaugaaacuggaucccuuuguuacacauuauacaaaaauuaauucaagauggauuaaaaacuuaaauguuagaccuaaaaccauaaaaacucuagaagaaaaccuaggcaauaccauucaggauagaggcaugggcaaagacuucaugucuaaaacaccaaaagcaauggcaacaaaagccaaaauugacaaaugggaucuaauuaaacuaaagagcuucugcacagcaaaagaaacuaccaucagagugaacaggcaaccuacagaauaggagaaaauuuuugcagucuacccaucugacaaagggcuaaaauccagaaucgacaaagaacucaaacaaauuuacaagaaaaaaucaaacaaccccaucaaaaaaugugcaaaguauaugaacagacacuuuucaaaagaagacauuuaugcagccaacaaacauaugaaaaaaugcucaucaucacuggccaucagagaaaugcaaaucaaaaccacagugagauaccaucucacaccaguuagaauggcaaucauuaaaaagucaggaaacaacaggugcuggagaggauguggagaaauaggaacaguuuuacacuguuggugggacuguacacuaguucaaccauuguggaagacaguguggcgauuccucagggaucuagaacuagaaauaccauuugacccagcuaucccauuacuggguauauacccaaaggaauauaaaucauacugcuauaaagacacaugcacacguauguuuacugcggcacuacucacaauaguaaagacuugggaccaacccaaauguccaacaaugauagacuggauuaagaaaauguggcauauauacaccauggaauacuaugcagccauaaaaaaugaugaguuccuguccuuuguagggacauggaugaagcuggaaaccaucauucugagcaaacccucacaaggacaaaaaaccaaacaccgcauguucucacucauaggugggaauugaacaaugagaacaccaggacacaggaaggggaacaucacacaccggggccuguugugggguggggggaggggugagggauagcauuaggagauauaccuaauguaaaugaugaguuaaugggugcagcaaaccaacauggcacauguauacauauguaacaaaccugcacguugugcacauguacccuagaacuuaaaguauaauaauaaaaaaaaauguauauuauaaaagaauguaagaaugugcauauuuauuuccuugcauauuaauggcauaagaaacuguuaacagggacuugggguaaggcuugugggaggaagguaguuuucacuguauuccuuuuguauuguuuuaaguuuuuacuuguuuuuuaagcaagcauguaucacuuuauaugauauuuaaaaguugcucuucucaggacagaaaaucauuuugauucauuucuaauucaaauaagcacuaauugaggauauuuuaauauauccucacauugugaaaggauuaaggcacaauuucuagcuucaaaacuguacaguaggcuaggguuaggaaccuuaacaauuagccacacugcaacauuggaaauuacugauuuucugaacauuaaguaguucaggcuuucuuacaugaaaguugaaacuugagcauuuuucuuuuuagaacuauauaaguaaaggauguaagauuuagauucucaguauuuagaaggguggagccgauuuaaagguagaaauaguuggcuuuuggcuuuugaaaugaagguugagaacaacugccaaguugcaagauuacuuccugagggucaguuguaugcaaagcaaauacaaggguaagaucagcuccuacauucaaaggguuuaaaaucuaggggacaggugggaaaaacagguacaugguaauacauggcagaaugugacaugugcuccaaaacagguacacaauucagauuauaaaggucauuucuagccgggcaugguggcuaaugccugguggcucacgguggccccaguacuuugggaggccgaggcggguggaucauuugaggucaggaguuugagaccggccugaccaacaugacgaaauaacaucucuacuaaaaauacaaaaaaauuagcugggccugguggugcacaucuguagucccaacuacccgggaggcugaggcagaagaaucacuugaaccugggaggcggagguugcacugagccaagaucacaccacugcacuccagccugggcacagagugagacucugucucaaaaacaaaaaaacaaaaaaacaaaaaaacauuuccaguugguaguaagaaaaggcuucauauaaguuguaguauuuaauauggagcuuaaaauaugugugaaaggugggaaggaagagcaucuuguaacuugccugaagaaagguguuauauuagaagggugucauaauaauuuauugagcccuuacuauguaccagauacuccauaugaacuaacucauugaauaaaacauaaaauaaaccuaugaaguaaccuccuggggagcaguucuccaaguguuucuauacauaugugacaacuguuguccuggacuaucuuuucaaaaauauuucuaugguuggccgggugcgguggcucaugcauguaaucccagcacuuugggaggccgaggcggguggauugcuugaggucaggaguucgagaccagccuggccgagauggugaaacccugucucugcuaaaaauacaaaaauuagccaggcacaguggcgggugccuguaaucccagcuacucgggaggcugaggcaggagaagcgcuugaacccgggaggcggaggcugcagugagccgagaucccgccacugcacucuaaccugggugacagagcaagacuccaacuaaaaaaaagaaaaaaaaauuuguauggcaaacacccugagaggcuagagaucaugucuuucuccaaugcagaggacagagucgcuuccugacuaggauaauaaagaaaaugucucccucuaggccaaagggugggcagccuugcugcagccuguuacgagacugaaagguuccuaagcucaggguucuucaacagugacacaaauccacugugugcacagaucuacccgggccauuuuccaucuaccccaugggauuuggggacaaggguaaccaaugggaacaugaagcucaugcugccuacugucuugugaauaaucaagucuuuugucucugacaugggaaucucaugccuucugcuaguuucugugaagauggggcaggcuaacuuguuagccuacaagcaggguaaaaucucagacccuuugcaguucuugacaaaaguacaauuauugucacuaucuauagagaaagaggcagagagauuugaugacuugccuaauauaguuaauaaauggaagaggagcugacauuccagcucaggcacucugucucuagaauugaacucuuaaccaguacaguaaacucuuauuaauuggccucagccuguuaugugucauggaaguacagucuguaaaagcaaaguggaguuuugccuggaauguuagacugagguauuuguaugaaauucuuaaggaagaaguguuuuuuuuuugguuuggaaaaagcaaagaaaucauguuguuuguuuguuugaaacccuccaaagauuuaucaucucacuuggaguaaaagccaaaguccuuauaagccuuaccucauauuaugguggacagccucuaaggugguccuuuaaugauccccacuucuugguauucacauucuguuuaauuaccucucuuugggcuuagugacuugcuucuaauaaacagaauauggcagcaagccagccuggccaacauggugaaacccugucucuacuaaaaauacaaaaauuagccaggcaugguggcgcaugccuguaaucccagcuacucaggaggcugaggcaggagaauugcuugaaccugggaggcagagguugcagugagcugagaucaccccaccgcacucuagcuugggcaacagaacgagacucugucucaaaaaaaaaaaaaaaaaaaaaaaaaaagaauauggcagcagugaugggccaucacuuuagagaugagguuugaaaaagacugugguuucuaucuugugugcuuucucucacucucuccaggggaagacagcugccacaugguaacgcaguccuguaaagagucccauguggugagagacgugguugggagugagcuuggaaguggauccccaacccucguccccaaacugagacuucacaugagacugcagcucuggccaacagcuucacuguaaccucacaacagaccuugagccacaagcacccagcuaagcugcagggagacccccugaaacuauugcuacggaauaaaagaugaaaugcuccugauuauuguaaauacaaaguugcaugcaggauuguguaaagacaaugccagguuggacugccagaaugagccaacugcacgugaugugauucccccugcagagagccuaugaauggacgugcagucagggagguuucacaucaccaagauuccuauccuagaaaggcagauguucauagcucuaggaauggaaugcgacccuuguggagagccuauaaacggaugcauggggguggggcgccuguccauauggauaagauagggcuagaaacgcccucaucuugccacagcucuucuaggccucuuuaggguuaaggcauacucccuucugagaauuucuggucuaacugguugucuagcuucacauccuguuuccguggauuguuuguaaccagcuuuuguugcaauuguuacugcugauuaauaucuugcuaaucauagguuauggaaagauugugcuucuguuuuaaggcucuguuagaaauuacugacgcacacacuauauuguaaauucuuaucucucuaugcuguacuucuacauacaaauguacuguacuucuacauacaaauguacuguacuucuacauacaaauguuauguuaauuacuucauccccaugugaccaucucaccucauaaucaaaugacccuaaaucccucacuaaccaacccccacccucacuuaacuuaauaauaaacgcugguauauccagagcauuguuggcaccaugggaccagaaggcggugaccccccuggacccagcuuucacuaucuugugugugucuauuauuucccaaccugcugaucugccugggaacaaagagagagccccauugcauugugggcugcugaccagauccugaagaugcaugcaacaacgagacagagaaauuugcugaaaaagcaccacagcaggaaacaacuaacacauccaaucaucaaaaagaagaaaagaccacgcuaacuccuuuacaacagacaauccagacagccauccugaacaacuugucuccagcgauccaggucuggcucaaccucugccuccuccugaugacacugauccuucuaccucuguuacgccacagacuguuaaaaacuauacauauugggccuauauuccuuuuccuccucuuauucgagccgugacauggauggaugcuccuaucgaggucuauguuaaugauaguauuuggaugccugguucucuagaugaucguuguccugcacaaccuucagaagaaggaaccccuuucaauaucacuuuagguuuuagguauccaccuuucugccugggacccacuaauguaugucucucauuagauauucaaacuugggcugucacacuaccgucuggucacucugucccuccuuuaggacacuugauaucagggcucucauuaaaaccucuaaggcagaucaaaacaguaaucacugauuauauucacacaucccaauauaagccuuuagaccuguguguccucucaacuugucuucaaaugcugacaaaauuaauauggaaagacuguguuagcucagaaggaaccguguuauuuaauucuucucacuacaccauuguugauugggcuccuaaaggucauauuacuaaugauugcucucaaggucacagagauugucaacauuuucucuauaauauuacuuaucaaaaacguagugacagcccuccccuauuauaucguagauuuaacuccuuuuuuccucuuaaguggaaaggggcagggguugcgccuccaaagccaaggcucauuguuccccacauaggaccugaacauuccaaauuauggagauuaacaauagcuaugacugguaugagaguuugggcuggagaaaguguuauaaguaaauccacuuugucaccucaaaauguauauacucucuauauggaguccaacaaaacuauaccacuuaaaaguuguguuaaaccaccauauauguuauuagcaggaaaguugcauauuaguucaaaaaccaacauaauuaccuguguuaauuguuacuuguauacuuguauugacucauccuuuaaucaauaucauaguauuuuaauagucagagccagacaagauauuuggcucccuguagccuuacauaggccuugggaaucuucccccuuuauccauguuauuaacaauauucuauagaaaauucuuaaaaggaguaaacaauuuauuuuuacauuaauugcaauaauaaugggcuugauugcuguuacugugauugcugcuacugcuggaguugcauuacguccaucuauucaaacuguucauuuuguggauaaauggcaaaaaaauucuacucggauguggaauucucagguauugaucaaaaauuggcuaaucaaauuaaugaucuaagacaaacuguuauauggaugggagguagaauuaugaguuuagaacauagauuacaaaugcaaugugauuggaauacuucugauuuuuguauaacuccauuucaauauaaugagucuguucacaauugggaaucaguaaaaugccauuuacaaggaagugaagauaauuuaaagcuuuagacauaagcaaacuaaaagaacagauuuuugaggccucucaagcacacuuaacugcuuuacccggugcugaaguuuuagacgguaucucugagggauuaucuaaucucaaccccauucaauagguaaaaucuuugggaggauccacuaucguuaauuuuguucuguguauaauuuguacuauugguuuauuguucauguguaaaauuggaaaaaauauucuucaguccaauugugaucagugccaagcuaugauugcuaugguucauuuaaaucagagagaagggggagauguaaggagacccccugaaacuguugcuauggaauaaaagaugaaaugcuccugauuauuguaaauacaaaguugcaugcaggauuguguaaagacaaugccagguuggacugccagaaugagccaacugcacgugaugugauucccccugcagagagccuaugaauggacgugcagucagggagguuucacaucaccaagauuccuauccuagaaaggcagauguucauagcucuaggaauggaaugcgacccuuguggagagccuauaaacggaugcauggggagguggcuguccguauggauaagauagggcuauaaacgcccucaucuugccacagcucuucuaggccucuuuaggguuaaggcauacucccuucugggaauuucuggucuaaccgguugucuagcuucacguccuguuuccauggauuguuuguaaccagcuuuuguugcaauuguuacugcugauuaauaucuugcuaaucauagguuauggaaagauuguguuucuguuuuaaggcucuguuagaaauuacugacacacacacuauauuguaaauucuuaucucucuauacuguacuucuacauacaaauguacuguacuucuacauacaaauguuaugugaaagaauuacuucauccccaugugaccaucucaccucauaaucaaaugacccuaaaucccucacuaaccuacccccacccucacuaaacuuaauaauaaacgcugguauauccagugcauuguuggcaccaugggaccggaaggcagugaacccccuggacccagcuuucacuaucuugugugugucuauuauuucucaaccugccaauacaccugggaacaaagagagagccccauugcauugugggcugcugaccagauccugcaauacuaaucugcguccagauuccugacccacagaaacugugauauaacaaauguuugccguuuuaagccacuaaguuuugguguaauuuguaagaagcaauagauaaugaauacauuccuccuccucccc

SEQ ID NO: 41 - RNA, 11621 nt, Homo sapiens
cggcggcuggcacagcggagcugcucuccaagcagcgggguucccaggcugggaaguuugcauugccguuucuuugcgggcgggcggcgagcgccgacugcaugccaccugcgagaggaagaggccgguaggugaacccgcagugacacuugcgcaggggacggaccagcucugcgcgcucugggauuaagucagggguugcuacuuccagcccaagcuugguaccucgccucgggcauuuucuugcagcuagaagggacgcaugccucuggcauaaauccaaccagagagucaccccucucaagcuagauuuuuuaaaaaucuagauauuauuuagaucauuucagcaaauucuuaaugcuuuggccuuucacaguaagauguugcuuaaucggcuggaucuccccacuccuugccaaggagacucaauuuugcaguugcccauaucugccuaguuaaaucguugcuauacuaaagguucugggaggguggggacagaauuuccccggugcuaaugcggcacugaaucgcaggaggcugcccaugcauuucuucagucaucuacaaccaagaauucucagagcagucccucggcagccuuuugaagcugugcuagagcagaaagcugcuauuguucucaucucucaacaaggaaagguucaaacuuugccucuucaauuugaaagauuuuuuuuuuauggugguggggggagggauugcaaucugauacucaaaguuaacuuugaggauuuggaguggucucccaguuuaagcugcagaucaaaucacagaugcccuaacgccugcaucucaagucgggggcacuucaucauuaauacgguggaguagucaaucacaagcaauuacaacguggguaaccuagccuggugaccgagcuaguuuacuuuccaaggaagugggaccauuuccucaacuggaauccuugaauuaauuuuaaacuuaaacuuccaaauuggaucaguauacaauacugguuguagaacuaggaggaauuuuuaaaaaucucaucuaauucucccucauuuggugggaggaggaaacagcauuuguaugauauguauccugccugcacaaugcugugagcuucaguagaugcucaauaaaugcucauugauguugggugguagaguggaauuguguguaccaaugucaucagcguaugacuggugccacagagcacacaaauguaguaggaagucagagaggauuggugaggacugggguggucaaaggaggcuuuaucaaggagcugggguuuggaggagacuuuaaggaccuacagaauuuuaacuuuaguuaauuuuagugcaaagaaaggaagaaggugaaggggaaagggacaguagaagaaagagaauuccacuucagugaaaaguugaacaacgaugaaguuuugggucuuugaucuguugguguggacaugguggggccagauuagagaagucagaacacggaguuugcccccaugcuguauacauuauaguaggaacuucuucugagcagguaaauaacgugaguucaauuccaugucugaaagcuucuucccuagaaggauuggcuugcagagucuggaaggauagaugcaaaugcaguuaccuaggugugggauggugaaaguuuugauugggaacuggugguaguagugaaaggaugacucagcauuuuaaaggggaaaaagagcaaaauuuggugacagguuggaauaagacauuaaauaaaggggacacucaaaaggacuucucuuuugagcaaauggugcuggggcaacugcauaucuacauguaaaacaaugaaauuggacccuuuccuuacaacacacacacaaauuaacuccaaucauagauuuaaauguaagaguuaaacucugaagcacuuagaagaaaacaaaggaggcugggugugguggcucacaccuguaaucccagcacuuugggaggccgaaucacaaggucaggaguucgagaccagccuggccaacauggugaaacucugucucuacuaaaaauacaaaaaauuagcugggcauaguggcggaugccuguaaucccagcuacucaggaggcugaggcaggagaaucgcuugaaccugggaggcggagguugcuguaagccaagaucaugccacugcacuacagcccgggugacagagugagacuccaucucaaaaaaaaaaaggaguaauccucaugaccuuaguuaggcaaaaccuucuuagaugugacaccaaaaacacaaaucauaaaagaaaaaauguucaauugaacuucaccacauuuaaaacuuuugugcuuuaaugaacacugucaagaaagugaagacaacucacagaauuggagaaaucauuugcaaauuauauaucuaauaaaggagcccuugcaacucaacaguaaaaagacaacccaauuuaaaaccggauaaaggauuuggauaaacauuuucccaaggaagauauacaaauggccaauaaacacaugaaaagaaacccaacaucauuauagccaucacugaaaagcaaauuaaaaccacagugagaggaaccccuucacaccuacuaggaugacuagauuauagaaaagauaguaaguauugguaaggauguggagaacuggaacccuuauacauugcuaauaggaauguaaaaugguacagaaaacaauugaaaaacaauguggcaauuccuccaaauguugaagauggaauuauaauucuacugcuagguagauacaaagagaauggaaaauacacauccacauaaagacuuguacaugaauguucauagcagcauuauucauaguagccaaaaguuggaaaccuagauguccaucuccugauaaauggaucauaucugauaaucuauguaaaggaauauuauucagcuauaaaaagggaugaaguaccgauacauguuacaauauggaugaaccuugaaaacaugcuaaaggaagccagucacaaaagucaccuauuguauguuuccauguauaugaaaugucuagaauagaaaaauccagagacggaaagcacauuaguggcugccagggacuugggggugggggaagagggaaugacugccaguggguaaggcuuuuugguggggguaaaaguaucuucaaaucgauuguggugaugguuauacaacucugugaauauauuaaaaaaaugguacaguuuaaauggaugaauuauaugauacaauaugguauguaauucuaucucaauaaagcuguuuaaaaauaacaucaaaaggaccccaagguuuuagccugugcggcaggaggaauagcucaguugcugacaaaagcaggagaacaaggaaaagagacagcugggaguuaagcagaaggacugaauucagcgugggcuguugaaucuaagauuuuggcuggcugcucgugaagaaacgccugcagaaagcuguaaaugccggcaguggcagugagugugauggacaaggcuaaagagaagacucaugaacuuucgguaaguagggagggcugaaaaagugagcucauaagagugaguggagaucaaacagguagagaaaagcuacaacaggauuuggagagaaugcacaaauauaagaugaacagaggaagagaagccaucaaagcagcccaagaaaauaacggugagagaggcaggagaaaacacagagaguaggggugggaggaugagcaagaccaaugugcuugcccaggcuggggaaggcgguggagagggccgcguggugagcaacaggaagagucuuuuugcuggagaggaagccuugaggguccauucgcagagccgagguggggaagaaggcagaagauucgaguuuaagaagcagugaggacagauagcagggcaagaaaaaaguuguguuuguguuuuaggaccugguaaaaacggggaagugguagguagggagccuagaaacuggccguuugcaguggaguguggguguuuccauuaaugaggagaggaacuucuuuuucccagccuggggugaagcaaguauagccccaggacauaacuucugaccuggggggagugaaacaaacucaaggagaagcugcugcuuuauugauuugaaaggacaagggcacagucguugcugggagugaaagggguggucacaaguucaaaagacaguaggaaacuguggcaggucagccgcauacacuugcuugugcugagucuuguccuguguuuuacuugaucacauucaagccuuacacguuuaaaucccaccuucucuuccacucugcugccauuuguauucccucagauaagaagguaaggagaaagggcugggugcaguggcucgugcuuguaaucccagcacuuugggagguggagguggacggaucacuugaagucaggaguucgagacuagccuggccaacauggugaaaucccaucucuaccaaaaauacaaaaaaauuagccagaugugcuggugggugccuauaaucccagcuacuugggaggcucaggcaggagaaucguuugaauccaggaggcggugguugcagugagccgagaucaugccacugcacuccagccugggcaacagaacgagacuccaucucgaaaaaaaaugguaaggagagcgggugccgugaccuggagcaaagggaggaguccugggugacaucugccucccauggcagggagguccuccgguaaaccugcuuagacacgcacaguugcuuucaacaguacugccauggacacgaaugaucuuuagguacagacaaggccuccuuacaagagacauauguuaggaaaagguagugccaaaauuccuuuauuaaguauauguuuuccuauuuagucauuaauguucauguuucaaaaugacccuguagcaaguaccacuugaugugagaaggaaucaaggcagguucauggauaaggcaugaacuuaaggucauggcaggagccugcagaagcucacaguacuguggcuuuguacucuguggcucugucaucuaucuuucuuucuaggcacaagaaaccaaaucuuuacugagugugguggaagaaugcccaggugagcacaggcacagugccaggugcugcagaggaccugagacaaagagucacugcgcccucagagaguuuagaauccagcacugaggaaaagagaggucauggggagcacaggugggucccacaaucaugggaagaauacuguuggcugcaagggccuggcaacuaucuaugacucaugaagaaacaggcccagugaaguaacguggcuccuugagaucacagagugaaagaacugagucucacauacauuauuugagacagguucucucucugucacccagguuggggugcagugguacaaucucggcucacugcaaccuccaucuuuugggcucaagcaauccucccaccucagccucccuaguagcuaggaccacaggcaugugccagcauauccggcuaacuuguuuuuuauuuucuuguggagacagggucucccuauguugcucaagcuggucucaaccuccugggaucaagugauucucccgccuuggccucccaaaauguugggauuauggaugugagccaccacacccagcuucaucauuuuuccagugaacauuuuucuagggaacaugaauauugaaagaaaagauucuccauguacuguuacccaucugugguggcauuuauuuccuaaaaagaaaugaaugugauaaaucuuucuuuuaggcugagcaauaauguuucacauauauaaaaucuugaaauuugaucuggauuauagcaauaaaauaaaacaugcauuaauauauaggagaauuacuuuggauacaaacccaacaaagauuuaaugaaaaacauugucauauaucaauaaaugcaaauaaagucugaaccccccccccccauuuccaccccucuaagucuagcaccccuagauggaggcggggguuggcuaacuuaggccggagagacgcaugcuggugaggucauggguuugcucuggucuguugguaguggucuuuugggggccacagccugcaugcugucucccacccagugugcuuguuugaccauguggcuggucgaccaugcugcugcuaccaaucacaacauggagucucauggaauccucucaaugacucuauuuuauagauggguaauuugcccaagaucccauaucuuguucuuggaauugagugcagguaauguuacuccagagggcaaccuuuuaaccaucagauuagagugccuccuugggugagugcaaaucucuuucuauuauuggaaaaacaauuaaacacguaugucagucugugguggaauuauuaaauaugaagucugggauauuuacuggccacauuacagguuguuuauuauuugccucauggugaaaaugucggucccacgugagauguuuguuaucugguuuuacccaaaagaaguaugggucuugaccucaaggauuauaguucagauccaugaugacuuuuucuuucacuucuagaguacucuuuucuucccccuaccccuugccccaagccaccagggggcacacagaauuggccauuuaccuuggaaaauuuaaggcaccaauuuuaaggauauagauaaauaucaguccaguaucuuuagagguacagcugggaauagacuaacacuaauaucagguaucuguccccauuuccauuuaaaagaucaucucuuuaaaauccuuuaggacugacaagugucucacccaugggccuuuuggucuucaggaggcaaugcccuuuuuauuuguguuucagugucaugguguaguuucaaguuggaucuguccucuuuccucugcaccuuucccugcagaaucacagagauauagauccucccuuccagggcaccagcaccaaucccccuuugccuuuguucucuucuccuucucuaggcuuuauucauggcugcucccugaugcccuucucauuuguuauuuuuuaaagguucuccuagguaacugcuacaagaggcucuaagccugcaccagaaucucugcuugucucuuguuuacuaugucauuagggcucauucacagugaacagaacccauucuaccugguuuaagcagaaagggacuucuuauaagauauuuaaugucuaggccaggcgcaguggcucaugccuguaaucccagcacuuugggaggccaagguagguggaucaccugaggucaggaguuugagaccagccuggccaacauagugaaacccugucucuacuaaaaaugcaaaaauuagcugagcaugguggugcacaccuauagucccagcuauuugggaagcugaagcaggagaaucacuugaacccaggaggcagagguugcagugagccaagauugugccacugcacuccagccuggaugacagugagacucugucucaaaaaaaaaaaaaaaaaaaaaaaaaauuaaugucuuacagaaugacuggagggcagaagaaacagaauugcuacugagcuuucaggcaugacucccagugccacaucacagacuuagguugacaagggagcugcuguucccgccaggaucaggagauuguggaaucagaaagcugccugccuccugggaagccauagugacagcugcccagcuucagacucaugccaccucuacuguuaucagacugaacuugggucugcccaccuagcacagcaaagccaaacauugacacugggauugcagcaagagaagugaggcauuuauuacaggaugccaagcaagaaggacugggcagcuaaagcuuaggaccugaacucccagaugguuugcaguuaaggguuuuuaaaggcagggaggaagauguuauaggcugcuaugguuugguuuugugucuccacccaaaucucauguugaacuguaauuccuauuguugggggagggacuuggugggaggugauuggaucauggggguggacuuccgccuugcuauucucaugguaaugaguucucacaagaucugguuguuuaaauauguguagcacuucccccuuugcuuucucucgccugcucuaccaugguaaggcaugcuugcuuccccuuugccuucugccaugauuguaaguuucuugagaccuuccagucauacuuccugucuagcccacagaacugugaaucaauuaaacuucuuuucuucauaaauuaccuagucucagaugucuugcucugucgcccaggcuggagugcaauggcgugaucuuggcucacugcaacauccaccucccugguucaagcgauuuaacugucucagccuccugaguagcugagauuacaugcaugugccaccaugccuggcuaauuuuuguauuuuuaguagagaccaguuuucaccauguuggucaggcuggucucgaacuccugacugcgugauccaucugccucggccucccaaagugcugggauuacaggcgugagccaccaugcccagccccuucagguacuucuuuauagcagugugagaauagacuaaugcacaggcaaaguuauaaaucaguucucacacugcuauaaagaacuaccugagacaguaauuuaugaagaaaagagguuuaauugauucacaguucugccaagaacugaguuugaacuugaaccaauuuaaaaacaccaaucagaaaucuugggaagacaaauugguuuggaaagugggaagaggggagguguuuccugggcauguauuuauuuugaagcucuagugggacguuccugaggagauaucagggagguaguuaguaauucagagcuggaacucuagagaaaagugaagcuggagacagauuugggagucaugugagagagagguagucaagggagugcacguuguaggaacucaauaaauguugguugaaugacauaaccccgucacagcugggaccagacuggcuaaaccauaauuaguguuuguagugagcagaaagagcacauugguuuuuggccauaaaggagaagugagggucuaggcauggagacuaggaauuaacaugaagcaacugcauaauuagcaaauguagcagauauuuggauugguucauuucagcuauuccaaucucuaagauagaaaacaaaaaaacuauauuuuccagauucccuugcagcuaggguuccauauauaauauuuagaucauacccaucagaugcagcguaccaaauuuggaaagcagaaguaaacaguuguaaggcaagaacugugcuuuggcuguuccuacuaguaagcacaguggugguaccuuugguuuuucuaugacagauguaacagguccuaagucccagcccuauauucauaguugucaagagauaagacacauaucaugguauugagauggaguagggacccccccagcauggaaauaaaggaaaaccuugaguuccuuuaaggguaauuccaagcaccuggcuagaacucagaaguaaaugagcaauuggauaagcgagaauguaauugucgcuuaaaauagccaaggaaguuagagucauaagaauguuugguucccuguagaaacuaaaaauaaccuuuuuuuaaaauuuuagauggagucuugcucugucccccaggcugaagugcaauggugcuaucucggcucacugcaauuuccgccucuuggguucaagcaauucuccugccucagccuccugaguagcugggauuaacgggcaccugucaacaugcccggccaauuuuugcauuuuuaguagagacaggguuucaccacguuggccaggcuggucuugaacuccugaccucgugauccuucugccucagccucccaaaagugcugggauuacagacgugagccaccgcaccuggcaaaaaucaccucuuaauauauguccuugaguuguuuuuuagaaaccuggaaccccacaaaauggauccccuggcaugcagaccucagucagauaagggggaacugaggacugaacucugaccacucuuccuuuuuucagauuucuuccuaaggggccucgaggaggucacacccacaagccagagcuaacauucuuacuacugauuccaaauuuuuagaugaagcuucgccuccuuaaccaauugcaaaucagaaaaucuuugaauuuaccuaugaccugugggcccccuuugagaugucccaccuuuuuaggucaaaccaauguauagcccaggagccaacaugugagggaagaagccuucaggugauccagccuccagcuguuggagccaccauagcugagaugccagauuucauggagcagagaugagccauccucucugugcacaguccaaauucuugacccagaaaaucuaugagcacauuaauggcuguuguuuuacuccacuaaguauagggugguuugcuauauagcaauaaggaaucaaaacaccccucacauucauguugcuuauuaucugaugguuaucuuucacugauaccaagcagcucauauuuuuaucuugcucaaaaaaccuuaaaugguucccccauugaauauggaauuaauuauauguucuuuaggcuugcauucaagauccugcucaguuuucaaucuuacuaccuuaucuuucacuuuaacccgcuacucacuuucaauccacuuguaccauaugucagaucuucacugggccacgacugaugcuuuccuaucccugugcccuuucaccagcacuggccuuuugcuuggaauugucguuuucuauauuuaccaaauuccuagcuaucauuccuucaagcucagugcaaaucucacauccuguaagacuuuguugcccaaugugguggccaguagccacauguggcuauuuacauuuaaaauagcacugauuaaaaucuaaaguuuaguuccucagucucacuggccacuuuccgugcucaggagccacaucccagugaacagcacugucuuuuuagcuuuuccuuucucaacuaaucucuccuucucccauauaaccuuaaaacugcauuuuuagcaauuuugaccugaauauuuuugaggguuguguaauccaaauccaugaaauuacagauacaccuuggcuuucuaccauucuagugcacugcucuguaaaugggagguggcucaaauauuaauugaguugggaggaagcagugguuaaucucuauacuauacuaaugggucugggugaaaugaccucuuaugaucuuuuccaguaaaauauucaacaaacaacauaagucuguuaucuucaacaauauauaaucuaucaggauagcuauuuuuuaaaucacaggugauuugggaggaaggucucuugagaggagcauucugccauucugguguagcuguuucuguucucuccagcagcuauuuuagguauuuaauuauuuuagagugugugcuugcauguguuuugucucaauguaugccaauuuuuugccuaguccuccugacaucuuaaucuaucugccaacgcuacaugccgucaagccaaggaagaauuuccuucucacuguugcuucucugaccaaggaagacauaagcacaaccaaucaccagacugaugaaauuccuuccaguuucagaaaagauuaucaccaaucccuacauuauaaaauucauacuaauaaagcacaaacauugaauucuaacau

SEQ ID NO: 42 - RNA, 21 nt, Homo sapiens
  misc_feature (1)..(21): Partial sequence of lncRNA12R tageted by siRNA
caguggcugguauuacaggaa

SEQ ID NO: 43 - RNA, 21 nt, Homo sapiens
  misc_feature (1)..(21): Partial sequence of lncRNA12R tageted by siRNA
aaggaaaaaguucuccauaaa

SEQ ID NO: 44 - RNA, 21 nt, Homo sapiens
  misc_feature (1)..(21): Partial sequence of lncRNA12R tageted by siRNA
uagggagaagauaaucagaua

SEQ ID NO: 45 - RNA, 21 nt, Homo sapiens
  misc_feature (1)..(21): Partial sequence of lncRNA13R tageted by siRNA
cccccccagcauggaaauaaa

SEQ ID NO: 46 - RNA, 21 nt, Homo sapiens
  misc_feature (1)..(21): Partial sequence of lncRNA13R tageted by siRNA
uuccaguuucagaaaagauua

SEQ ID NO: 47 - RNA, 25 nt, Homo sapiens
  misc_feature (1)..(25): Partial sequence of lncRNA8R tageted by siRNA
ccaguccacugaaccacauccagaa

SEQ ID NO: 48 - RNA, 25 nt, Homo sapiens
  misc_feature (1)..(25): Partial sequence of lncRNA8R tageted by siRNA
gaacaguguucacauucgcuccuau

SEQ ID NO: 49 - RNA, 21 nt, Homo sapiens
  misc_feature (1)..(21): Partial sequence of lncRNA12R tageted by siRNA
gcaaaucaguguuggccaucu

SEQ ID NO: 50 - RNA, 21 nt, Homo sapiens
  misc_feature (1)..(21): Partial sequence of lncRNA12R tageted by siRNA
ggugucuacauggcagcauaa

(19) United States
(12) Patent Application Publication
(10) Pub. No.: US 2015/0329858 A1
(43) Pub. Date: Nov. 19, 2015

(54) LONG NON-CODING RNA USED FOR ANTICANCER THERAPY

(71) Applicants: THE UNIVERSITY OF TOKYO, Tokyo (JP); KYOWA HAKKO KIRIN CO., LTD., Tokyo (JP)

(72) Inventors: Hiroyuki ABURATANI, Tokyo (JP); Aya NONAKA, Tokyo (JP); Tatsuya MIYAZAWA, Machida-shi, Tokyo (JP); Tetsuo YOSHIDA, Machida-shi, Tokyo (JP)

(21) Appl. No.: 14/442,732

(22) PCT Filed: Nov. 15, 2013
(86) PCT No.: PCT/JP2013/080878

§ 371 Date: May 14, 2015

Related U.S. Application Data
(60) Provisional application No. 61/727,185, filed on Nov. 16, 2012.

(51) Int. Cl. C12N 15/113; C12Q 1/68

(52) U.S. Cl. CPC C12N 15/113; C12Q 1/6886; C12N 2310/14; C12Q 2600/136; C12Q 2600/178; C12Q 2600/158; C12N 2320/30

(57) ABSTRACT

The present invention provides a novel long non-coding RNA (lncRNA), which is induced by β-catenin and highly expressed in cancer, a nucleic acid that suppresses expression of the lncRNA, a means for promoting or suppressing cell growth by using the lncRNA or the nucleic acid, and the like.

### TECHNICAL FIELD

The present invention relates to a long non-coding RNA (lncRNA) induced by β-catenin in cancer cells, and showing anti-cancer cell activity by suppression of expression by nucleic acid and the like, nucleic acid used for suppressing expression of lncRNA, and the like.

### BACKGROUND ART

It is known that Wnt signal is closely involved in the development, differentiation and growth of cell, and β-catenin is activated and the expression of the target gene is regulated in a cell stimulated by Wnt ligand. Also, it is widely known that abnormality of Wnt signal induces cell canceration and promotes growth and differentiation of cancer cells, and metastasis and infiltration of cancer cells.

In recent years, the correlation between expression of lncRNA such as HOTAIR and the like and poor treatment prognosis of high-grade malignant cancer and the like has been reported. HOTAIR is suggested to control methylation modification of histone via Polycomb complex in cancer cells such as breast cancer and the like (non-patent document 1).

Polycomb complex is constituted of factors including histone methylation modifying enzyme EZH2, and involved in the development, differentiation and growth control of cells. In addition, the correlation between malignancy and EZH2 expression has been suggested in plural cancer types such as lymphoma and breast cancer (non-patent document 2).

Along with the development of a high-speed sequencer, new search for lncRNA has been tried mainly in human and mouse cells. Recently, large scale sequence analyses in an attempt to obtain lncRNA that binds to a Polycomb complex in mouse ES cells and human colorectal cancer cell lines have been reported (non-patent documents 3-5, patent document 1).

On the contrary, however, most of such lncRNAs are only structure predictions in silico, and many remain to be elucidated as to the functions such as relation to the growth, differentiation and metastasis of cancer cells and the like. In addition, lncRNA induced by β-catenin has not been known yet.

### DOCUMENT LIST

### Patent Document

patent document 1: WO 2012/065143

### Non-Patent Documents

non-patent document 1: Gupta et al. (2010) Nature 464, 1071-1076.
non-patent document 2: Chase and Cross. (2011) Clinical Cancer Research 17, 2613-2618.
non-patent document 3: Zhao et al. (2010) Molecular Cell 40, 939-953.
non-patent document 4: Chu et al. (2011) Molecular Cell 44, 667-678.
non-patent document 5: Guil et al. (2012) Nature Structural & Molecular Biology doi:10.1038/nsmb.2315

### SUMMARY OF THE INVENTION

### Problems to be Solved by the Invention

From the aspects of cancer therapy, improving effects on the growth and metastasis of high-grade malignant cancer and poor treatment prognosis are desired. For this end, a superior target and increased specificity for the target provide an effective means.

The present invention aims to provide a novel target of cancer and nucleic acid for the treatment of cancer.

### Means of Solving the Problems

The present inventors have obtained a novel lncRNA, which is induced by β-catenin, by performing a large-scale base sequence analysis of expressed RNA by using a high-speed sequencer in metastatic cancer cells, and found that a strong anti-cancer cell activity can be exhibited by suppressing expression of the above-mentioned lncRNA by using nucleic acid and the like.

Accordingly, the present invention provides the following invention that solves the aforementioned problem.

(1) An lncRNA consisting of a nucleotide sequence having not less than 80% identity with the nucleotide sequence shown in any of SEQ ID NOs: 1-15 and SEQ ID NOs: 38-41.
(2) An lncRNA that hybridizes to a complementary strand of a nucleic acid consisting of the nucleotide sequence shown in any of SEQ ID NOs: 1-15 and SEQ ID NOs: 38-41 under stringent conditions.
(3) An lncRNA consisting of the nucleotide sequence shown in any of SEQ ID NOs: 1-15 and SEQ ID NOs: 38-41.
(4) A nucleic acid consisting of a nucleotide sequence complementary to the lncRNA of any one of the above-mentioned (1)-(3).
(5) A double-stranded nucleic acid consisting of the lncRNA of any one of the above-mentioned (1)-(3), and a nucleic acid consisting of a nucleotide sequence complementary to the nucleotide sequence of the lncRNA.
(6) A nucleic acid that suppresses expression of the lncRNA of any one of the above-mentioned (1)-(3).
(7) The nucleic acid of the above-mentioned (6), which is selected from siRNA, antisense nucleic acid, shRNA or miRNA.
(8) The nucleic acid of the above-mentioned (6), which is an siRNA targeting the nucleotide sequence shown in any of SEQ ID NOs: 42-50.
(9) A vector expressing the nucleic acid or lncRNA of any one of the above-mentioned (1)-(8).
(10) A cell introduced with the nucleic acid or lncRNA of any one of the above-mentioned (1)-(8).
(11) A cell introduced with the vector of the above-mentioned (9).
(12) A cell growth promoter or growth inhibitor comprising the nucleic acid or lncRNA of any one of the above-mentioned (1)-(8) as an active ingredient.
(13) A diagnostic drug or a therapeutic drug for a disease caused by abnormality in cell growth, which comprises the nucleic acid or lncRNA of any one of the above-mentioned (1)-(8) as an active ingredient.
(14) The diagnostic drug or therapeutic drug of the above-mentioned (13), wherein the disease is selected from gastrointestinal cancer, liver cancer, kidney cancer, lung cancer, skin cancer, breast cancer, uterine cancer, prostate cancer, urinary bladder cancer, or head and neck cancer.
(15) A method of detecting expression of lncRNA, comprising using the lncRNA of any one of the above-mentioned (1)-(3).
(16) A method of detecting a mutation of lncRNA, comprising using the lncRNA of any one of the above-mentioned (1)-(3).
(17) A method of suppressing expression of lncRNA, comprising using the nucleic acid of any one of the above-mentioned (4)-(8).
(18) A method of screening for a substance that suppresses expression or function of lncRNA, comprising using the lncRNA of any one of the above-mentioned (1)-(3).

### Effect of the Invention

According to the present invention, growth, metastasis and infiltration of cancer cells that express target lncRNA can be suppressed. Using expression of target lncRNA as an index, moreover, it is possible to specify, diagnose and treat metastatic cancer cells.

### BRIEF DESCRIPTION OF THE DRAWINGS

FIG. 1, (a) shows expression levels of lncRNA8R when siRNAs (siRNA1-3) for β-catenin were introduced into SW480 cells, (b) shows expression levels of lncRNA9R when siRNAs (siRNA1-3) for β-catenin were introduced into SW480 cells, (c) shows expression levels of lncRNA12R when siRNAs (siRNA1-3) for β-catenin were introduced into SW480 cells, (d) shows expression levels of lncRNA13R when siRNAs (siRNA1-3) for β-catenin were introduced into SW480 cells, and (e) shows expression levels of β-catenin when siRNAs (siRNA1-3) for 3-catenin were introduced into SW480 cells.

FIG. 2 shows signal values of lncRNA8R in normal large intestine clinical samples, colorectal cancer cell line samples and colorectal cancer clinical samples.

FIG. 3 shows signal values of lncRNA9R in normal large intestine clinical samples, colorectal cancer cell line samples and colorectal cancer clinical samples.

FIG. 4 shows signal values of lncRNA12R in normal large intestine clinical samples, colorectal cancer cell line samples and colorectal cancer clinical samples.

FIG. 5 shows signal values of lncRNA13R in normal large intestine clinical samples, colorectal cancer cell line samples and colorectal cancer clinical samples.

FIG. 6 shows anticellular activity when siRNA for lncRNA8R was introduced into SW480 cells, wherein the dotted line shows anticellular activity of control siRNA-introduced cells, the solid line shows anticellular activity of 8R1siRNA-introduced cells, and the broken line shows anticellular activity of 8R2siRNA-introduced cells.

FIG. 7 shows anticellular activity when siRNA for lncRNA12R was introduced into SW480 cells, wherein the dotted line shows anticellular activity of control siRNA-introduced cells, the solid line shows anticellular activity of 12R#16siRNA-introduced cells, and the broken line shows anticellular activity of 12R#17siRNA-introduced cells.

FIG. 8 shows anticellular activity when siRNAs for lncRNA12R, lncRNA13R were each introduced into SW480 cells and SW620 cells.

FIG. 9 shows RNA immunoprecipitation of SW480 cells by using anti-PRC2 antibodies (EZH2, SUZ12), wherein net-like pattern shows lncRNA9R, black shows lncRNA12R, white shows TUG′, gray shows MALAT1, vertical line shows HOTAIR, diagonal line shows ACTS, and shade shows SNORD15.

FIG. 10 shows RNA immunoprecipitation of SW620 cells by using anti-PRC2 antibody (SUZ12).

FIG. 11 shows colony-formability when siRNA for lncRNA12R and siRNA for lncRNA13R were each introduced into SW480 cells and SW620 cells.

FIG. 12 shows migratory ability when siRNA for lncRNA12R was introduced into SW480 cells.

### MODE FOR CARRYING OUT THE INVENTION

The lncRNA in the present invention is a long single-stranded RNA induced by β-catenin, which is a novel lncRNA highly expressed in cancer.

As the lncRNA of the present invention, lncRNA consisting of a nucleotide sequence having not less than 80% identity with the nucleotide sequence shown in any of SEQ ID NOs: 1-15 and SEQ ID NOs: 38-41, more preferably lncRNA consisting of a nucleotide sequence having not less than 90% identity, most preferably lncRNA consisting of a nucleotide sequence having not less than 95% (e.g., not less than 96%, not less than 97%, not less than 98%, not less than 99%) identity can be mentioned. The identity of the nucleotide sequences in the present invention can be calculated using homology calculation algorithm NCBI BLAST (National Center for Biotechnology Information Basic Local Alignment Search Tool) under the following conditions (expectancy=10; gap allowed; filtering=ON; match score=1; mismatch score=−3).

As the lncRNA of the present invention, lncRNA that hybridizes to a complementary strand of lncRNA consisting of the nucleotide sequence shown in any of SEQ ID NOs: 1-15 and SEQ ID NOs: 38-41 under stringent conditions can be mentioned. To be specific, lncRNA consisting of the nucleotide sequence shown in any of SEQ ID NOs: 1-15 and SEQ ID NOs: 38-41 can be mentioned.

In the present invention, lncRNA that hybridizes under stringent conditions includes, for example, lncRNA that can be identified by using a nucleic acid (including double-stranded nucleic acid such as cDNA, cRNA and the like) complementary to lncRNA having the nucleotide sequence shown in any of SEQ ID NOs: 1-15 and SEQ ID NOs: 38-41 or a partial fragment thereof as a probe, adding a probe RNA labeled with γ-³²P-ATP to a hybridization buffer composed of 20×SSC 7.5 mL, 1M Na₂HPO₄ (pH 7.2) 0.6 mL, 10% SDS 21 mL, 50×Denhardt's solution 0.6 mL, 10 mg/mL sonicated salmon sperm DNA 0.3 mL, reacting the mixture at 50° C. overnight, washing same with 5×SSC/5% SDS solution at 50° C. for 10 min, further washing same with 1×SSC/1% SDS solution at 50° C. for 10 min, thereafter taking out the membrane, and exposing same to an X-ray film.

As the nucleic acid that suppresses expression of lncRNA in the present invention, any nucleic acid such as a single-stranded nucleic acid, a double-stranded nucleic acid and the like can be used as long as it is a nucleic acid containing a partial nucleotide sequence of lncRNA and/or a nucleotide sequence complementary to the nucleotide sequence and suppressing the expression of lncRNA, and a double-stranded nucleic acid is preferably used. As used herein, “suppressing the expression” is used in the meaning encompassing suppression of transcription of lncRNA of the present invention (e.g., antigene), cleavage of lncRNA (e.g., siRNA, shRNA, ribozyme), or inhibition of formation of functional lncRNA (e.g., antisense nucleic acid, miRNA).

While the partial nucleotide sequence of lncRNA to be the target sequence for the nucleic acid of the present invention is not particularly limited, for example, when the sequence of siRNA and/or shRNA is to be designed, it can be searched for by using a searching software provided on various web sites. Examples of such site include, but are not limited to, siRNA Target Finder (http://www.ambion.com/jp/techlib/misc/siRNA_finder.html) and Insert Design Tool for the pSilencer (registered trade mark) Expression Vectors (http://www.ambion.com/jp/techlib/misc/psilencer_converter.html) provided by Ambion, and GeneSeer (http://codex.cshl.edu/scripts/newsearchhairpin.cgi) provided by RNAi Codex.

In the present invention, the double-stranded nucleic acid refers to a nucleic acid wherein two strands form a pair to have a double strand forming part. The double strand forming part refers to a part wherein nucleotide or a derivative thereof constituting a double-stranded nucleic acid constitutes base pairs to form a double strand. The double strand forming part generally contains 15-27 base pairs, preferably 15-25 base pairs, more preferably 15-23 base pairs, further preferably 15-21 base pairs, particularly preferably 15-19 base pairs.

A single-stranded nucleic acid constituting a double-stranded nucleic acid is generally composed of 15-30 bases, preferably 15-29 bases, more preferably 15-27 bases, further preferably 15-25 bases, particularly preferably 17-23 bases, most preferably 19-21 bases.

When the double-stranded nucleic acid of the present invention has an additional nucleotide or a nucleotide derivative that does not form a double strand on the 3′-side or 5′-side following the double strand forming part, these overhanging parts may be a ribonucleotide, a deoxyribonucleotide or a derivative thereof.

A double-stranded nucleic acid having an overhanging part composed of 1-4 bases, generally 1-3 bases, preferably two bases and more preferably dTdT or UU at the 3′-end or 5′-end of at least one of the strands is used. The overhanging part can be formed on antisense strand alone, sense strand alone, or both antisense strand and sense strand. A double-stranded nucleic acid having an overhanging part on both antisense strand and sense strand is preferably used. As used herein, the “sense strand” means a strand having a sequence homologous to a target sequence of lncRNA, and the “antisense strand” means a strand having a sequence complementary to the target sequence. In addition, a sequence continued from the double strand forming part, which is partially or fully identical with the target sequence, or a sequence continued from the double strand forming part, which is identical with a base sequence of a complementary strand of the target sequence can also be used. As the double-stranded nucleic acid of the present invention, a nucleic acid molecule that forms the above-mentioned double-stranded nucleic acid by the action of ribonuclease, for example, such as Dicer and the like (WO 2005/089287), a double-stranded nucleic acid free of a 3′-end or 5′-end overhanging part and the like can also be used.

As the nucleic acid of the present invention, moreover, a single-stranded nucleic acid can also be used. Such nucleic acid having a suppressive activity on lncRNA expression, wherein 1-3 bases, preferably 1-2 bases, more preferably 1 base, is/are substituted, deleted or added, can also be used. In addition, nucleic acids of not more than 30 bases, preferably not more than 29 bases, more preferably not more than 27 bases, further preferably not more than 25 bases, particularly preferably not more than 23 bases, including the above nucleic acid, can be mentioned.

In addition, the sense strand and antisense strand of the above-mentioned double-stranded nucleic acid may be linked via a spacer sequence to give a single-stranded nucleic acid. Such single-stranded nucleic acid is preferably a single-stranded nucleic acid such as shRNA having a double strand forming part due to a stem loop structure and the like. A single-stranded nucleic acid having a stem loop structure generally has a 50-70 base length.

As another single-stranded nucleic acid, an antisense nucleic acid can be mentioned. The antisense nucleic acid may be DNA or RNA, or DNA/RNA chimera. When the antisense nucleic acid is DNA, RNA:DNA hybrid formed by target RNA and antisense DNA is recognized by endogenous RNase H and can cause selective degradation of the target RNA.

The nucleic acid of the present invention may be a nucleic acid having not more than 70 base length, preferably not more than 50 base length, more preferably not more than 30 base length, which is designed to form the above-mentioned single-stranded nucleic acid or double-stranded nucleic acid by the action of ribonuclease and the like.

The molecule constituting the nucleic acid of the present invention may be any molecule as long as it is a molecule wherein nucleotides or molecules having function equivalent to that of the nucleotide are polymerized, and examples thereof include RNA which is a polymer of ribonucleotides, DNA which is a polymer of deoxyribonucleotides, chimeric nucleic acid composed of RNA and DNA, and a nucleotide polymer wherein at least one nucleotide for these nucleic acids is substituted by a molecule having function equivalent to that of the nucleotide. siRNA, sh (short hairpin) RNA, miRNA and a derivative thereof containing at least one molecule having function equivalent to that of nucleotide therein for these nucleic acids are also included in the nucleic acid of the present invention. Uracil (U) in RNA can be unambiguously interpreted as thymine (T) in DNA.

Examples of the molecule having function equivalent to that of nucleotide include nucleotide derivative and the like. The nucleotide derivative may be any molecule as long as it is a molecule obtained by modifying nucleotide and, for example, a molecule obtained by modifying ribonucleotide or deoxyribonucleotide and the like are preferably used to improve or stabilize nuclease resistance, increase affinity to complementary strand nucleic acid, increase cell permeability, or visualize, as compared to RNA or DNA.

Examples of the nucleotide derivative include sugar moiety-modified nucleotide, phosphodiester bond-modified nucleotide, base-modified nucleotide, nucleotide wherein at least one of the sugar moiety, phosphodiester bond and base is modified, and the like.

As the sugar moiety-modified nucleotide, any can be used as long as a part of or whole chemical structure of the sugar of nucleotide is modified with or substituted by any substituent, or substituted by any atom, and 2′-modified nucleotide is preferably used.

Examples of the 2′-modified nucleotide include 2′-modified nucleotide wherein a 2′-OH group of ribose is substituted by substituent(s) selected from the group consisting of H, OR, R, R′ OR, SH, SR, NH₂, NHR, NR₂, N₃, CN, F, Cl, Br and I (R is alkyl or aryl, preferably alkyl having 1-6 carbon atoms, and R′ is alkylene, preferably alkylene having 1-6 carbon atoms), and the 2′-OH group is preferably substituted by F or a methoxy group. Examples thereof also include 2′-modified nucleotide substituted by substituent(s) selected from the group consisting of a 2-(methoxy)ethoxy group, a 3-aminopropoxy group, a 2-[(N,N-dimethylamino)oxy]ethoxy group, a 3-(N,N-dimethylamino)propoxy group, a 2-[2-(N,N-dimethylamino)ethoxy]ethoxy group, a 2-(methylamino)-2-oxoethoxy group, a 2-(N-methylcarbamoyl)ethoxy group and a 2-cyanoetoxy group, and the like.

Examples of the sugar moiety-modified nucleotide include bridged artificial nucleic acid (Bridged Nucleic Acid, BNA) having two cyclic structures by introduction of a bridged structure into the sugar moiety, and specific examples thereof include locked artificial nucleic acid (Locked Nucleic Acid, LNA) wherein the 2′-position oxygen atom and the 4′-position carbon atom are bridged via methylene, ethylene bridged artificial nucleic acid (Ethylene bridged nucleic acid, ENA) [Nucleic Acid Research, 32, e175(2004)] and the like. Furthermore, peptide nucleic acid (PNA) [Acc. Chem. Res., 32, 624 (1999)], oxypeptide nucleic acid (OPNA) [J. Am. Chem. Soc., 123, 4653 (2001)], peptide ribonucleic acid (PRNA) [J. Am. Chem. Soc., 122, 6900 (2000)] and the like can also be mentioned.

The phosphodiester bond-modified nucleotide may be any as long as a part of or whole chemical structure of the phosphodiester bond of nucleotide is modified with or substituted by any substituent, or substituted by any atom. Examples thereof include a nucleotide wherein a phosphodiester bond is substituted by a phosphorothioate bond, a nucleotide wherein a phosphodiester bond is substituted by a phosphorodithioate bond, a nucleotide wherein a phosphodiester bond is substituted by an alkylphosphonate bond, a nucleotide wherein a phosphodiester bond is substituted by a phosphoroamidate bond and the like.

The base-modified nucleotide may be any as long as a part of or whole chemical structure of the base of nucleotide is modified with or substituted by any substituent, or substituted by any atom. Examples thereof include a nucleotide wherein an oxygen atom in the base is substituted by a sulfur atom, a nucleotide wherein a hydrogen atom is substituted by an alkyl group having 1-6 carbon atoms, a nucleotide wherein a methyl group is substituted by hydrogen or an alkyl group having 2-6 carbon atoms, a nucleotide wherein an amino group is protected by a protecting group such as an alkyl group having 1-6 carbon atoms, an alkanoyl group having 1-6 and the like.

Furthermore, examples of the nucleotide derivative include a nucleotide derivative wherein other chemical substance such as lipid, phospholipid, phenazine, folate, phenanthridine, anthraquinone, acridine, fluorescein, rhodamine, coumarin, pigment and the like is added to nucleotide or nucleotide derivative, in which at least one of sugar moiety, phosphodiester bond and base is modified. Specific examples thereof include 5′-polyamine-added nucleotide derivative, cholesterol-added nucleotide derivative, steroid-added nucleotide derivative, bile acid-added nucleotide derivative, vitamin-added nucleotide derivative, Cy5-added nucleotide derivative, Cy3-added nucleotide derivative, 6-FAM-added nucleotide derivative, biotin-added nucleotide derivative and the like.

In addition, the nucleotide derivative may form a bridged structure such as alkylene structure, peptide structure, nucleotide structure, ether structure, ester structure, and a structure combining at least one of these and the like with other nucleotide or nucleotide derivative in the nucleic acid.

The nucleic acid of the present invention may be constituted of any nucleotide or a derivative thereof as long as it is a nucleic acid having function equivalent to that of a nucleic acid consisting of a partial nucleotide sequence of lncRNA or a nucleic acid consisting of a nucleotide sequence complementary to the nucleotide sequence of the nucleic acid. That is, a nucleic acid consisting of a partial nucleotide sequence of lncRNA or a nucleic acid consisting of a nucleotide sequence complementary to the nucleotide sequence of the nucleic acid may be a nucleic acid wherein the nucleotide constituting the nucleotide sequence is substituted by ribonucleotide, deoxyribonucleotide or a derivative thereof, which has function equivalent to that of the nucleotide.

A production method of the nucleic acid of the present invention is not particularly limited, and examples thereof include a method using known chemical synthesis, an enzymatic transcription method and the like. Examples of the method using known chemical synthesis include phosphoramidite method, phosphorothioate method, phosphotriester method, CEM method [Nucleic Acid Research, 35, 3287 (2007)] and the like. For example, it can be synthesized by ABI3900 High Throughput Nucleic Acid Synthesizer (manufactured by Applied Biosystems). After completion of the synthesis, dissociation from the solid phase, removal of protecting group, purification of the object product and the like are performed. By purification, a nucleic acid with a purity of not less than 90%, preferably not less than 95%, is desirably obtained. In the case of a double-stranded nucleic acid, synthesized and purified sense strand and antisense strand may be mixed at a suitable ratio, for example, 0.1-10 equivalents, preferably 0.5-2 equivalents, more preferably 0.9-1.1 equivalents, further preferably an equimolar amount, of sense strand is mixed with 1 equivalent of antisense strand and the mixture may be annealed before use, or may be directly used by omitting the step of annealing the mixture. Annealing may be performed under any conditions as long as a double-stranded nucleic acid can be formed. Generally, it includes mixing nearly equimolar amounts of the sense strand and the antisense strand, heating the mixture at about 94° C. for about 5 min, and slowly cooling to room temperature. As an enzymatic transcription method for production of the nucleic acid of the present invention, a method including transcription using a plasmid or DNA having the object nucleotide sequence as a template, and phage RNA polymerase, for example, T7, T3, or SP6 RNA polymerase can be mentioned.

Examples of the method for introduction of the nucleic acid of the present invention into a cell include a method using a carrier for transfection, preferably a cationic carrier such as cationic liposome and the like, calcium phosphate method, electroporation method, microinjection method and the like.

It is also possible to use, instead of the nucleic acid of the present invention, a vector capable of expressing the nucleic acid by introduction into the cell. To be specific, the nucleic acid and the like can be expressed by inserting a sequence encoding the nucleic acid of the present invention into the downstream of a promoter in an expression vector to construct an expression vector, and introducing same into the cell.

As an expression vector, a recombinant virus vector produced by inserting a sequence encoding the nucleic acid of the present invention into the downstream of a promoter in a virus vector, and introducing the vector into a packaging cell can be used. Examples of the virus vector include retrovirus vector, lentivirus vector, adenovirus vector, adeno-associated virus vector, Sendai virus vector and the like.

Expression of lncRNA can be suppressed by introducing such single-stranded nucleic acid or double-stranded nucleic acid into the cell.

Also, the lncRNA expression suppressive activity of the single-stranded nucleic acid or double-stranded nucleic acid of the present invention can be evaluated by transfecting the nucleic acid and the like into cultured cancer cells and the like by using a cationic liposome and the like, culturing same for a given time, and quantifying the expression level of lncRNA in the cancer cells by RT-PCR. The suppressive effect on cell proliferation can be evaluated by calculating the viable cell number of the cells introduced with the single-stranded nucleic acid or double-stranded nucleic acid of the present invention.

The method for detecting the expression of lncRNA of the present invention may be any as long as the presence of lncRNA in a sample can be detected. Examples thereof include (1) Northern hybridization [Science 294, 853-858 (2001)], (2) dot blot hybridization [Molecular Cloning, 3rd ed.], (3) in situ hybridization [Methods in Enzymology, 254, 419 (1995)], (4) quantitative PCR [Nucleic Acids Research, 32, e43 (2004)], (5) differential hybridization [Trends Genet., 7, 314 (1991)], (6) microarray [Genome Res., 6, 639 (1996)], (7) ribonuclease protection assay [mirVana miRNA Detection Kit (manufactured by Ambion)] and the like.

The method for detecting a mutation of lncRNA of the present invention may be any as long as a mutation of the nucleotide sequence of lncRNA in a sample can be detected. Examples thereof include a method of detecting a heteroduplex formed by hybridization of a nucleic acid having a non-mutant nucleotide sequence and a nucleic acid having a mutant nucleotide sequence, a method of detecting the presence or absence of mutation by directly sequencing a nucleotide sequence derived from a sample and the like.

Examples of the method of detecting a heteroduplex include methods such as (1) heteroduplex detection method by polyacrylamide gel electrophoresis [Trends genet., 7, 5 (1991)], (2) single strand conformation polymorphism analysis method [Genomics, 16, 325-332 (1993)], (3) chemical cleavage of mismatches (CCM) [Human Genetics (1996), Tom Strachan and Andrew P. Read, BIOS Scientific Publishers Limited], (4) enzymatical cleavage of mismatches [Nature Genetics, 9, 103-104 (1996)], (5) denaturing gel electrophoresis [Mutat. Res., 288, 103-112 (1993)] and the like.

As a method of screening for a substance that promotes or suppresses expression or function of lncRNA by using lncRNA of the present invention, for example, a substance that promotes or suppresses expression or function of lncRNA can be screened for by selecting a nucleotide sequence as a screening target from the nucleotide sequence of lncRNA of the present invention, and utilizing a cell expressing a nucleic acid having the nucleotide sequence.

As a cell expressing a nucleic acid having the nucleotide sequence of lncRNA, which is used for screening, a transformed cell obtained by introducing a vector expressing a nucleic acid having the nucleotide sequence into a host cell such as an animal cell and the like, a cell directly introduced with a nucleic acid having the nucleotide sequence without using a vector and the like can also be used.

As a specific screening method, a method using, as an index, changes in the expression level of lncRNA to be the screening target can be mentioned.

A test substance is contacted with a cell expressing a nucleic acid having the nucleotide sequence, and a substance that promotes or suppresses expression of lncRNA is obtained by using changes in the expression level of the nucleic acid as an index.

The present invention also relates to a pharmaceutical composition containing, as an active ingredient, a nucleic acid such as single-stranded nucleic acid, double-stranded nucleic acid and the like or a vector that suppresses expression of the above-mentioned lncRNA of the present invention. The pharmaceutical composition can further contain a carrier effective for intracellular transfer of the nucleic acid. The pharmaceutical composition of the present invention can be used for the treatment or prophylaxis of cancer diseases. Examples of the cancer include solid tumors such as gastrointestinal cancer, liver cancer, kidney cancer, lung cancer, skin cancer, breast cancer, uterine cancer, prostate cancer, urinary bladder cancer, head and neck cancer and the like.

Examples of the carrier effective for intracellular transfer of a nucleic acid include a cationic carrier. Examples of the cationic carrier include cationic liposome, cationic polymer and the like. In addition, as a carrier effective for intracellular transfer of a nucleic acid, a carrier utilizing a virus envelope may be used. As the cationic liposome, a liposome containing 2-O-(2-diethylaminoethyl)carbamoyl-1,3-O-dioleoylglycerol (hereinafter to be also referred to as liposome A), Oligofectamine (Invitrogen), Lipofectine (Invitrogen), Lipofectamine (Invitrogen), Lipofectamine 2000 (Invitrogen), DMRIE-C (Invitrogen), GeneSilencer (Gene Therapy Systems, Inc.), TransMessenger (QIAGEN), TransIT TKO (Mirus Bio LLC) and the like are preferably used. As the cationic polymer, JetSI (Qbiogene), Jet-PEI (polyethylenimine; Qbiogene) and the like are preferably used. As a carrier utilizing a virus envelope, GenomeOne (HVJ-E liposome; Ishihara Sangyo Kaisha, Ltd.) and the like are preferably used.

A composition containing a single-stranded nucleic acid, double-stranded nucleic acid or vector to be contained in the pharmaceutical composition of the present invention and the above-mentioned carrier can be prepared by a method known to those of ordinary skill in the art. For example, it can be prepared by mixing a suitable concentration of a carrier dispersion and a single-stranded nucleic acid, double-stranded nucleic acid or vector solution. When a cationic carrier is used, the composition can be prepared with ease by mixing in an aqueous solution by a conventional method, since a single-stranded nucleic acid, double-stranded nucleic acid or vector is negatively charged in an aqueous solution. Examples of the aqueous solvent used for the preparation of the composition include water for injection, distilled water for injection, electrolyte fluids such as saline and the like, sugar solutions such as glucose solution, maltose solution and the like, and the like.

The conditions of pH, temperature and the like for the preparation of the composition can be appropriately selected by those of ordinary skill in the art. For example, liposome A can be prepared by gradually adding oligo double-stranded RNA solution in 10% aqueous maltose solution to 16 mg/ml liposome dispersion in 10% aqueous maltose solution at pH 7.4, 25° C. with stirring.

The composition can be processed into a uniform composition where necessary by performing a dispersion treatment using an ultrasonic dispersion apparatus, a high-pressure emulsification apparatus and the like. An optimal method and conditions for the preparation of a composition containing a carrier and a single-stranded nucleic acid, double-stranded nucleic acid or vector vary depending on the carrier to be used, and those of ordinary skill in the art can select an optimal method for the carrier to be used, without being caught by the above-mentioned methods.

As the pharmaceutical composition of the present invention, a liposome comprised of complex particles comprising a single-stranded nucleic acid, double-stranded nucleic acid or vector and a lead particle as constituent components, and a lipid membrane for coating the complex particles, wherein constituent components of the lipid membrane can be solved in a polar organic solvent, and wherein the polar organic solvent can be contained in a liquid at such a concentration that the constituent components of the lipid membrane are dispersible and the complex particles are dispersible is also used preferably. Examples of the lead particle include a fine particle containing lipid assembly, liposome, emulsion particle, polymer, metal colloid, fine particle preparation and the like as a constituent component, and preferable examples thereof include a fine particle containing a liposome as a constituent component. The lead particle in the present invention may contain, as a constituent component, a complex of a combination of two or more from lipid assembly, liposome, emulsion particle, polymer, metal colloid, fine particle preparation and the like, and may contain, as a constituent component, a complex of a combination of lipid assembly, liposome, emulsion particle, polymer, metal colloid, fine particle preparation and the like, and other compound (e.g., sugar, lipid, inorganic compound etc.).

Examples of the lipid membrane for coating the complex particles include those containing neutral lipid and polyethylene glycol-phosphatidylethanolamine and the like as constituent components.

The liposome can be prepared according to the method described in, for example, WO 2006/080118 and the like.

A suitable mixing ratio of a single-stranded nucleic acid, double-stranded nucleic acid or vector and a carrier to be contained in the pharmaceutical composition of the present invention is 1-200 parts by weight of a carrier per 1 part by weight of a single-stranded nucleic acid, double-stranded nucleic acid or vector. It is preferably 2.5-100 parts by weight, more preferably 10-20 parts by weight, of a carrier per 1 part by weight of a single-stranded nucleic acid, double-stranded nucleic acid or vector.

The pharmaceutical composition of the present invention may contain, besides the above-mentioned carrier, a pharmaceutically acceptable carrier, a diluent and the like. A pharmaceutically acceptable carrier, a diluent and the like are essentially chemically inert and harmless compositions and do not at all affect the biological activity of the pharmaceutical composition of the present invention. Examples of such carrier or diluent include, but are not limited to, a salt solution, a sugar solution, a glycerol solution, ethanol and the like.

The pharmaceutical composition of the present invention contains the complex in an amount effective for the treatment or prophylaxis of diseases, and is provided in a form permitting appropriate administration to the patients. The formulation of the pharmaceutical composition of the present invention may be, for example, a liquid such as injection, eye drop, inhalation and the like, or an external preparation such as ointment, lotion and the like.

In the case of a liquid, the concentration range of the pharmaceutical composition of the present invention is generally 0.001-25%(w/v), preferably 0.01-5%(w/v), more preferably 0.1-2%(w/v). The pharmaceutical composition of the present invention may contain an adequate amount of any pharmaceutically acceptable additive, for example, emulsifying aid, stabilizer, isotonizing agent, pH adjuster and the like. Any pharmaceutically acceptable additive can be added in a suitable step, which may be before or after dispersing of the complex.

A freeze-dried formulation can be prepared by a dispersion treatment of a single-stranded nucleic acid, double-stranded nucleic acid or vector and a carrier, and a freeze-drying treatment thereafter. The freeze-drying treatment can be performed by a conventional method. For example, a given amount of a complex solution after the above-mentioned dispersion treatment is dispensed to a vial container under sterile conditions, predried at about −40 to −20° C. for about 2 hr, primarily dried at about 0-10° C. under reduced pressure, then secondarily dried at about 15-25° C. under reduced pressure for lyophilization. Then, for example, the inside of the vial is purged with nitrogen gas, and the vial is capped to give a freeze-dried formulation of the pharmaceutical composition of the present invention.

The pharmaceutical composition of the present invention can be redissolved by the addition of any suitable solution and used. Examples of such solution include water for injection, electrolyte fluids such as saline and the like, glucose solution, other conventional infusion solutions and the like. While the liquid amount of the solution varies depending on the use and the like and is not particularly limited, 0.5- to 2-times the amount before freeze-drying, or not more than 500 ml, is preferable.

The pharmaceutical composition of the present invention can be administered to animals including human by, for example, intravenous administration, intraarterial administration, oral administration, intratissue administration, transdermal administration, transmucosal administration or transrectal administration, and is preferably administered by an appropriate method according to the symptom of patients. In particular, intravenous administration, transdermal administration, and transmucosal administration are preferably used. It can also be locally administered by, for example, local administration into cancer and the like. Examples of the dosage form suitable for these administration methods include various injections, oral preparations, drip infusions, absorbents, eye drops, ointments, lotions, suppositories and the like.

While the dose of the pharmaceutical composition of the present invention is desirably determined in consideration of the drug, dosage form, condition of patient such as age, body weight and the like, administration route, nature and level of diseases and the like, it is generally 0.1 mg-10 g/day, preferably 1 mg-500 mg/day, for an adult, in the mass of a single-stranded nucleic acid, double-stranded nucleic acid or vector. In some cases, an amount not more than this may be sufficient, or conversely, a dose not less than this may be necessary. It can be administered one to several times per day, and can also be administered at an interval of one day-several days.

The present invention is explained in the following by referring to Examples, which are not to be construed as limitative.

### EXAMPLES

### Example 1

### Obtainment of Novel lncRNA Induced by β-Catenin in Cancer Cell

1-1 Identification Method of Novel lncRNA

To colorectal cancer cell line SW480 (3×10⁵ cells) was added 20 nM each of siRNA (Life Technologies, Stealth RNAi 1299003) for β-catenin or control siRNA (Life Technologies, Stealth RNAiNegative control 12935-112) by using HiPerFect Transfection Reagent (QUIAGEN) and according to the attached protocol, and total RNA was recovered by using TRIzol (Life Technologies) 48 hr later. Each RNA sample was prepared according to Directional mRNA-seq sample preparation protocol (Illumina, Inc.) and analyzed using Genome Analyzer IIx, and RNA expression in all genomic regions was confirmed. The analysis software used was TOPHAT analysis (Bioinformatics, 2009, 25 (9) p 1105).

As a result, a region where RNA expression level decreases due to siRNA for β-catenin, which is accompanied by a region within 5 kb from said region where binding of β-catenin can be confirmed, was identified as a transcription product under regulation of β-catenin. The binding of β-catenin was confirmed by ChIP-seq. ChIP followed a method of using the anti-β-catenin antibody of Santa Cruz Biotechnology, Inc. (sc-7199 and Cancer Sci. 2008, 99 (6) p 1139). For sequence analysis, Genome Analyzer IIx and ChIP-seq Sample Prep kit (Illumina, Inc.) were used. The analysis software used was Model-based Analysis for ChIP-seq (MACS, Genome Biol (2008) vol. 9 (9) pp. R137).

1-2 Full-Length Nucleotide Sequence of Novel lncRNA

Using Marathon cDNA Amplification Kit (Clontech Laboratories, Inc.), the terminal sequence of lncRNA identified above was determined. In addition, using Hiseq2000 and Paired-End mRNA-seq kit (Illumina, Inc.), sequence analysis was performed, cDNA was cloned by TOPHAT analysis, and the full-length nucleotide sequence of lncRNA (SEQ ID NOs: 1-15 and SEQ ID NOs: 38-41) was determined. The splicing variants corresponding to the respective SEQ ID NOs were named as follows.

SEQ ID NO: 1: 7F, SEQ ID NO: 2: 8F Variant 1, SEQ ID NO: 3: 8F Variant 2, SEQ ID NO: 4: 8F Variant 3, SEQ ID NO: 5: 8F Variant 4, SEQ ID NO: 6: 8R Variant 1, SEQ ID NO: 7: 8R Variant 2, SEQ ID NO: 8: 9R, SEQ ID NO: 9: 12R Variant 1, SEQ ID NO: 10: 12R Variant 2, SEQ ID NO: 11: 13R Variant 1, SEQ ID NO: 12: 13R Variant 2, SEQ ID NO: 13: 13R Variant 3, SEQ ID NO: 14: 14R Variant 1, SEQ ID NO: 15: 14R Variant 2, SEQ ID NO: 38: 12R Variant 3, SEQ ID NO: 39: 12R Variant 4, SEQ ID NO: 40: 12R Variant 5, SEQ ID NO: 41: 13R Variant 3

### Example 2

### Induction of Novel lncRNA by β-Catenin

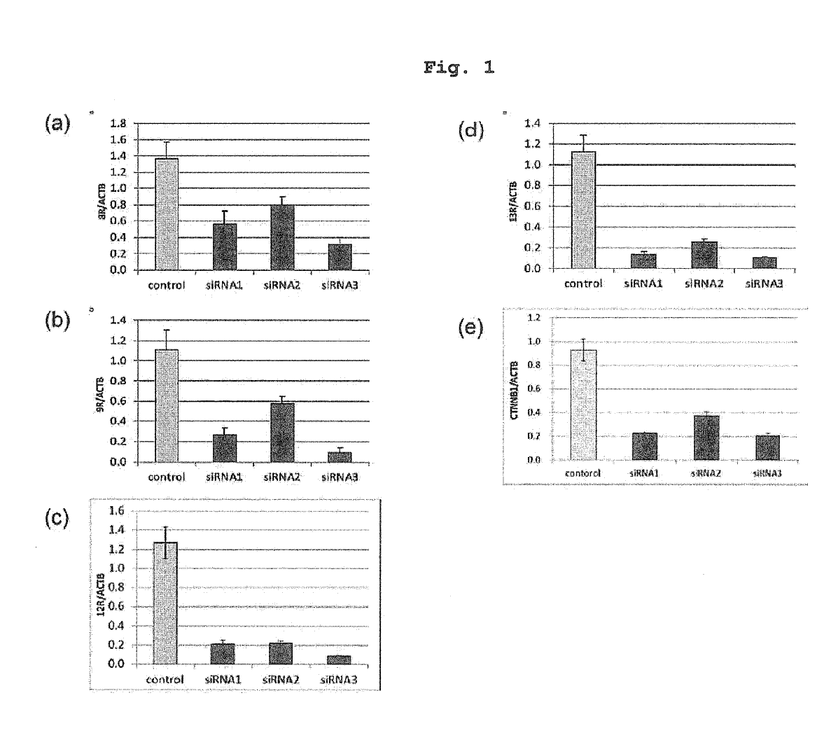

To confirm that novel lncRNA obtained in Example 1 is induced by β-catenin, according to the method of Example 1-1, siRNA for β-catenin or control siRNA was added to colorectal cancer cell line SW480 and the mixture was cultured, after which total RNA was recovered, and the expression level of novel lncRNA shown in Example 1 and β-catenin was each measured by quantitative RT-PCR method. The primers used are shown in Table 1. Primers 8RF and 8RR (SEQ ID NOs: 16, 17) were used for the detection of lncRNA8R, primers 9RF and 9RR (SEQ ID NOs: 18, 19) were used for the detection of lncRNA9R, primers 12RF and 12RR (SEQ ID NOs: 20, 21) were used for the detection of lncRNA12R, and primers 13RF and 13RR (SEQ ID NOs: 22, 23) were used for the detection of lncRNA13R. As a result, the expressions of lncRNA decreased along with decreasing expression of β-catenin in lncRNA8R, lncRNA9R, lncRNA12R and lncRNA13R (FIG. 1).

TABLE 1

| SEQ ID | Sequence | |
|---|---|---|
| No. | 5′—3′ | Name |
| 16 | CTGGGTGGCTCCTCTCAACC | 8RF |
| 17 | CCAAAGGGACCCACATCGAC | 8RR |
| 18 | GCTGATCCCAGGCCCTACCT | 9RF |
| 19 | GAATGCCTCCCGGTCCTTCT | 9RR |
| 20 | CCCAACCACGTCTCTCACCA | 12RF |
| 21 | TTCAAAGAGCACAGCTGCACA | 12RR |
| 22 | GATTCAACAGCCCACGCTGA | 13RF |
| 23 | ATGCCACCTGCGAGAGGAAG | 13RR |
| 28 | GACGGAGGTTGAGATGAAGC | MALAT1_F |
| 29 | ATTCGGGGCTCTGTAGTCCT | MALAT1_R |
| 30 | TCCCGGAGGTGCTCTCAATC | HOTAIR_F |
| 31 | GGGCTCCCTCTCTCCACTCC | HOTAIR_R |
| 32 | TTGCCCAGGTGGCCTACTCT | SNORD15A_F |
| 33 | CCTTCTCAGACAAATGCCTCTAAGT | SNORD15A_R |
| 34 | AGAAGGAGATCACTGCCCTGGCACC | ACTB_F |
| 35 | CCTGCTTGCTGATCCACATCTGCTG | ACTB_R |
| 36 | CAAGCACTACCACCAGCACTGTTAC | TUG1_F |
| 37 | GCAATCAGGAGGCACAGGACATAAT | TUG1_R |

### Example 3

### Expressions of lncRNA8R, lncRNA9R, lncRNA12R and lncRNA13R in Cancer Cells and Cancer Tissues

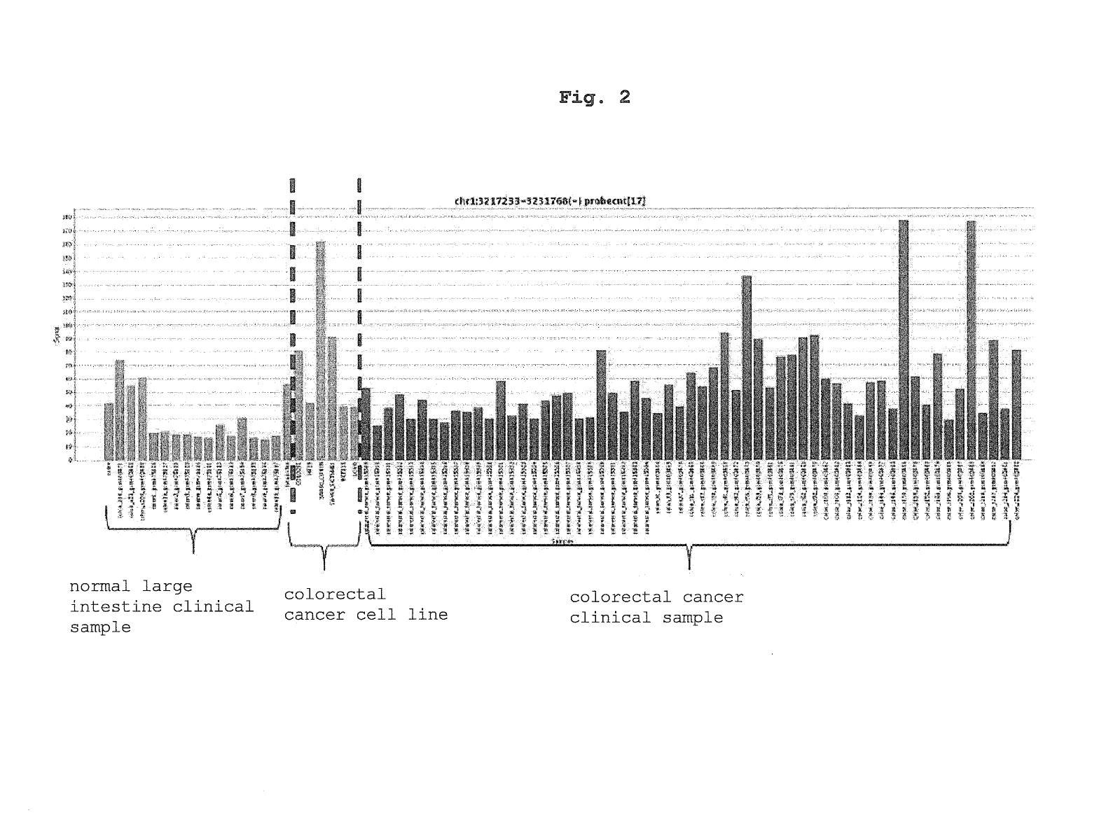

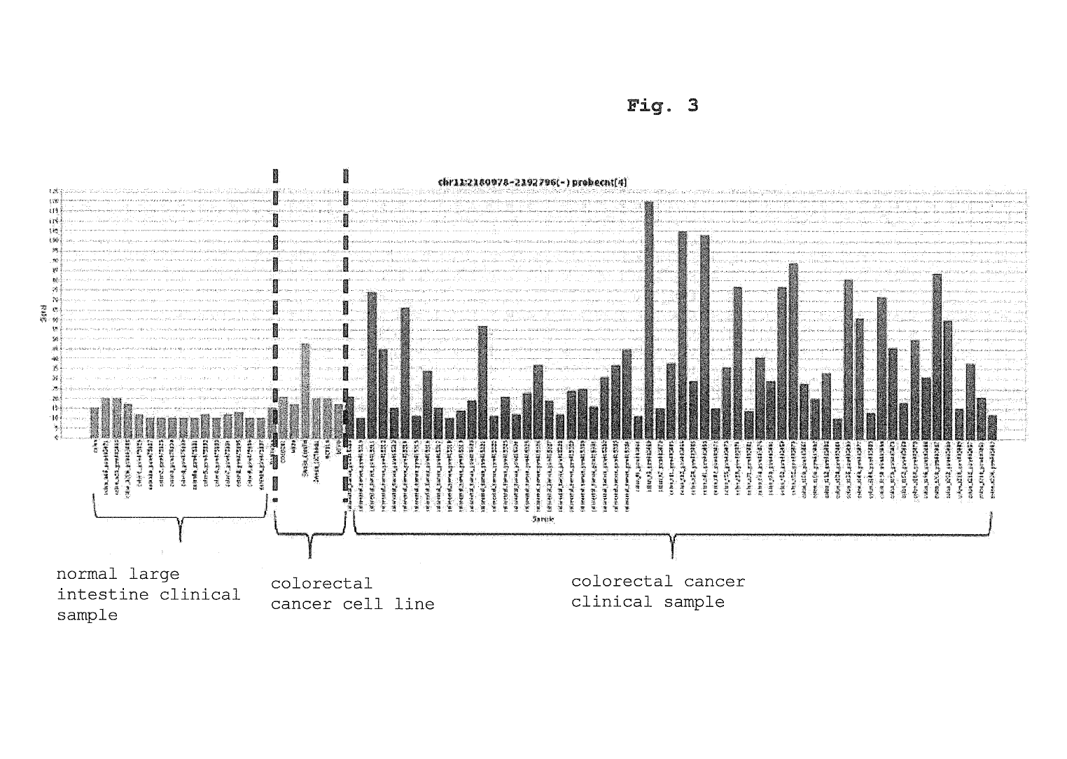

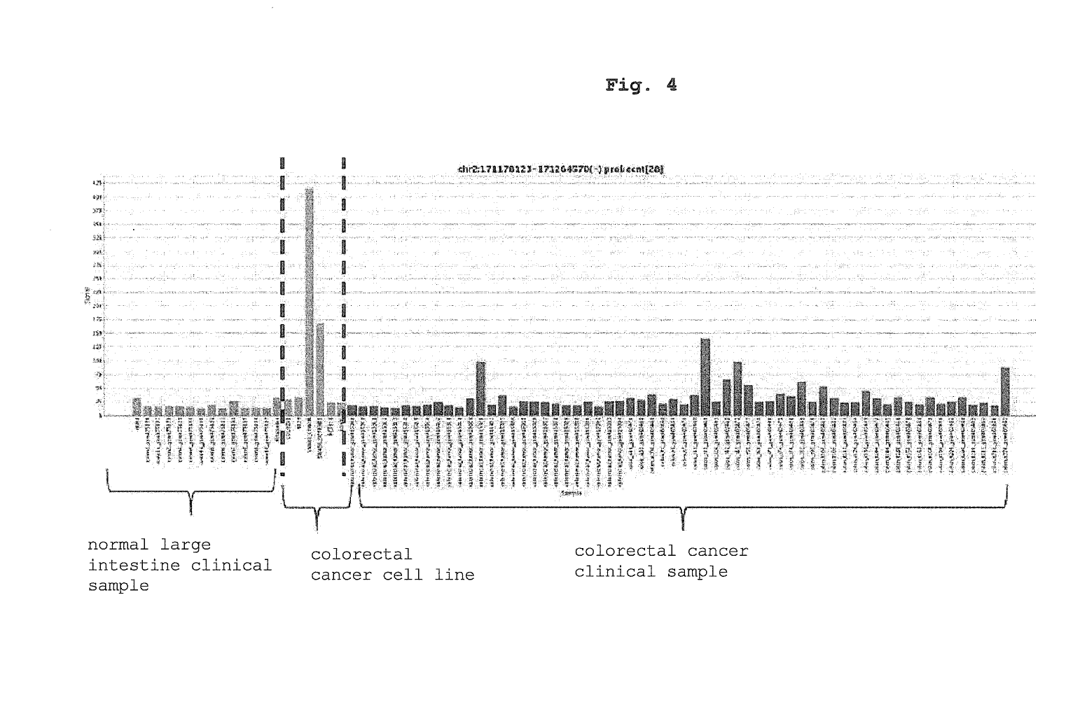

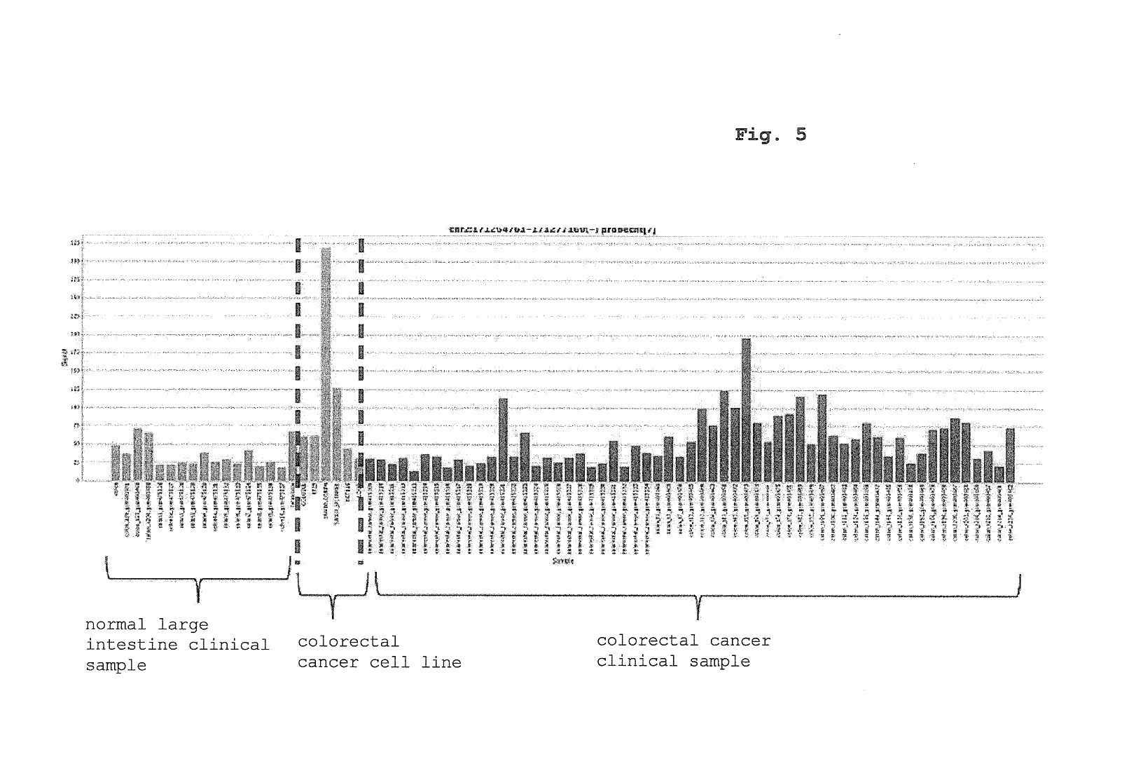

The data of public experimental results “GSE23768” and “Series GSE16125” using Human Exon 1.0 ST Array (Affymetrix, Inc.) were obtained from Gene Expression Omnibus (GEO) (National Center for Biotechnology and Information (NCBI)), and the expression of lncRNA8R, lncRNA9R, lncRNA12R and lncRNA13R in cancer cells and cancer tissues was analyzed. The geometric mean of signal values for 17 probes designed in the minus(−) strand of human chromosome region “chromosome 1: 3217233-3231768” of lncRNA8R obtained in Example 1, the geometric mean of signal values for 4 probes designed in the minus(−) strand of human chromosome region “chromosome 11: 2181184-2192608” of lncRNA9R, the geometric mean of signal values for 28 probes designed in the minus(−) strand of human chromosome region “chromosome 2: 171178123-171264570” of lncRNA12R and the geometric mean of signal values for 7 probes designed in the minus(−) strand of human chromosome region “chromosome 2: 171264761-171277160” of lncRNA13R were calculated. As a result, it was confirmed that all expressions were promoted in colorectal cancer samples (FIGS. 2-5).

### Example 4

### Suppressive Effect of Cell Growth by Suppressing Expression of lncRNA8R, lncRNA12R and lncRNA13R

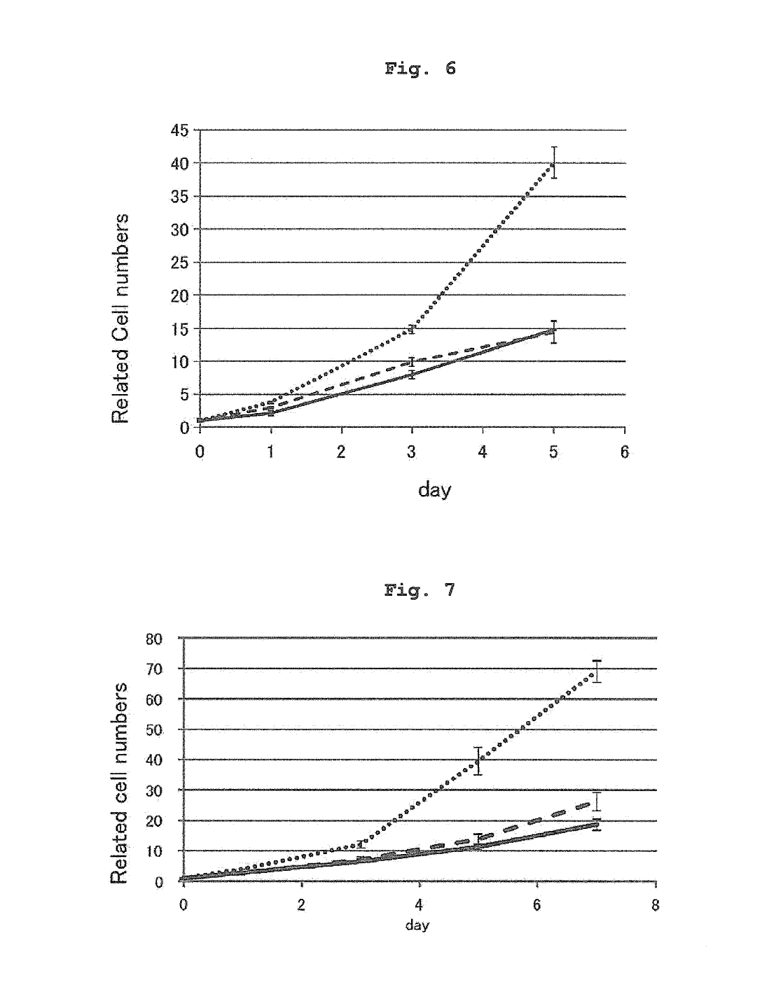

siRNA (Stealth RNAi(8R1, 8R2), Life Technologies) for lncRNA8R obtained in Example 1 was introduced into colorectal cancer cell line SW480 by a method similar to that in Example 1. The sequence of siRNA used and the sequence of its target lncRNA8R are shown in Table 2 (SEQ ID NOs: 24 and 47, 25 and 48). As control siRNA, Stealth RNAi Negative Control Medium GC duplex#2 (Life Technologies, 12935-112) was used. Cell proliferation was measured using Cell Counting Kit-8 (Dojindo Laboratories) from day 0 after siRNA introduction. As a result, the growth of colorectal cancer cells was suppressed by decreasing the expression of lncRNA8R (FIG. 6).

In addition, using lentivirus vector pLK0.1 puro lentivirus vector (Addgene), shRNAs (12R#16, 12R#17) for lncRNA12R obtained in Example 1 were each introduced into colorectal cancer cell line SW480 by a conventional method. The sequence of oligo DNA used to design shRNA used and the sequence of its target lncRNA12R are shown in Table 3 (SEQ ID NOs: 26 and 49, 27 and 50). Cell proliferation was measured using Cell Counting Kit-8 (Dojindo Laboratories) from day 0 after introduction. As a result, the growth of colorectal cancer cells was suppressed by decreasing the expression of lncRNA12R (FIG. 7).

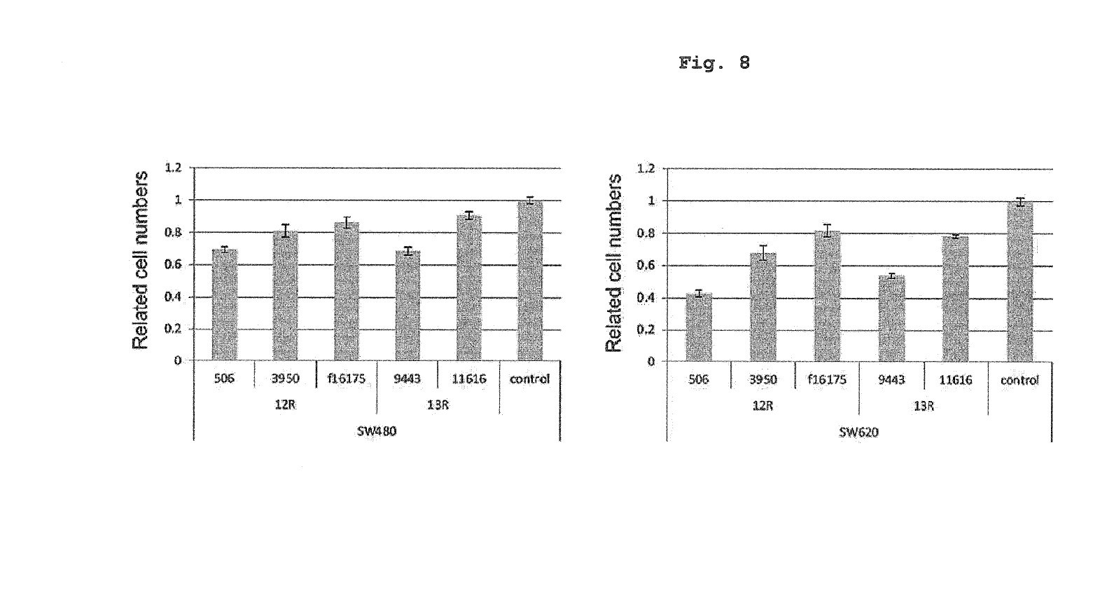

Furthermore, various siRNAs for lncRNA12R and lncRNA13R (custom synthesized siRNAs, GeneDesign Inc.) were each introduced into colorectal cancer cell line SW480 and colorectal cancer-derived lymph node metastasis cell line SW620 (1.2×10⁴ cells) to a final concentration of 50 nM by using Lipofectamine 2000 (Life Technologies) and according to the attached protocol. The target sequences of siRNA used are shown in Table 4 (SEQ ID NOs: 42-46). The control siRNA used was AllStars Negative Control siRNA (QUIAGEN, 1027281). After 72 hr of siRNA introduction, the viable cell number was measured using CellTiter-Glo Luminescent Cell Viability Assay kit (Promega Corporation), the anticellular activity was evaluated by calculating the survival rate relative to control siRNA. As a result, the growth of colorectal cancer cells was suppressed by decreasing the expression of lncRNA12R and lncRNA13R (FIG. 8).

From the above, a suppressive effect on the cell growth by the suppression of expression of lncRNA8R, lncRNA12R and lncRNA13R was confirmed.

TABLE 2

| SEQ ID | Sequence | |
|---|---|---|
| No. | 5′—3′ | Name |
| 24 | UUCUGGAUGUGGUUCAGUGGACUGG | 8R1 |
| 26 | AUAGGAGCGAAUGUGAACACUGUUC | 8R2 |
| 47 | CCAGUCCACUGAACCACAUCCAGAA | SR1target |
| 48 | GAACAGUGUUCACAUUCGCUCCUAU | SR2target |

TABLE 3

| SEQ ID | Sequence | |
|---|---|---|
| No. | 5′—3′ | Name |
| 26 | GCAAATCAGTGTTGGCCATCT | 12R#16 |
| 27 | GGTGTCTACATGGCAGCATAA | 12B#17 |
| 49 | GCAAAUCAGUGUUGGCCAUCU | 12R#16target |
| 50 | GGUGUCUACAUGGCAGCAUAA | 12R#17target |

TABLE 4

| SEQ ID | Sequence | |
|---|---|---|
| No. | 5′—3′ | Name |
| 42 | CAGUGGCUGGUAUUACAGGAA | 12R#506 |
| 43 | AAGGAAAAAGUUCUCCAUAAA | 12R#3950 |
| 44 | UAGGGAGAAGAUAAUCAGAUA | 12R#f16175 |
| 45 | CCCCCCCAGCAUGGAAAUAAA | 13R#9443 |
| 46 | UUCCAGUUUCAGAAAAGAUUA | 13R#11616 |

### Example 5

### Binding of Novel lncRNA to PRC2 in Cancer Cells

To show that novel lncRNA obtained in Example 1 binds to Polycomb Recessive Complex 2 (PRC2), RNA chromatin immunoprecipitation (RIP-ChIP) experiment using antibodies against EZH2 and SUZ12, which are the constituent components of PRC2, was performed. A cell extract was prepared from SW480 cell line, control IgG (Sigma-Aldrich Corporation, catalog No. A-6154), anti-EZH2 antibody (Active Motif, catalog No. 39933), and anti-SUZ12 antibody (Abcam plc, catalog No. ab12073) were used. The method followed Nature Protocol 2006 vol 1 NO12011.12.1.

cDNA was prepared from the obtained RNA by using SuperScript III (Invitrogen). Quantitative RT-PCR was performed using specific primers to each of lncRNA9R and lncRNA12R obtained in Example 1, and TUG1, MALAT1, HOTAIR, ACTB and SNORD15, which are known lncRNAs. The primer sequences used are shown in Table 1 (SEQ ID NOs: 18-21, 28-37). As a result, it was shown that lncRNA9R and lncRNA12R coprecipitate with SUZ12 antibody and EZH2 antibody and bind to PRC2 (FIG. 9).

Furthermore, to comprehensively clarify ncRNA binding to PRC2 in cancer cell line, RNA chromatin immunoprecipitation-sequencing (RIP-seq) experiment was performed using an antibody against SUZ12 which is the constituent component of PRC2. A nuclear extract was prepared from SW620 cell line, and control IgG (Sigma-Aldrich Corporation, catalog No. A-6154), and an anti-SUZ12 antibody (Abcam plc, catalog No. ab12073) were used. RIP method followed partly-altered Nature Protocol 2006 vol 1 NO12011.12.1.

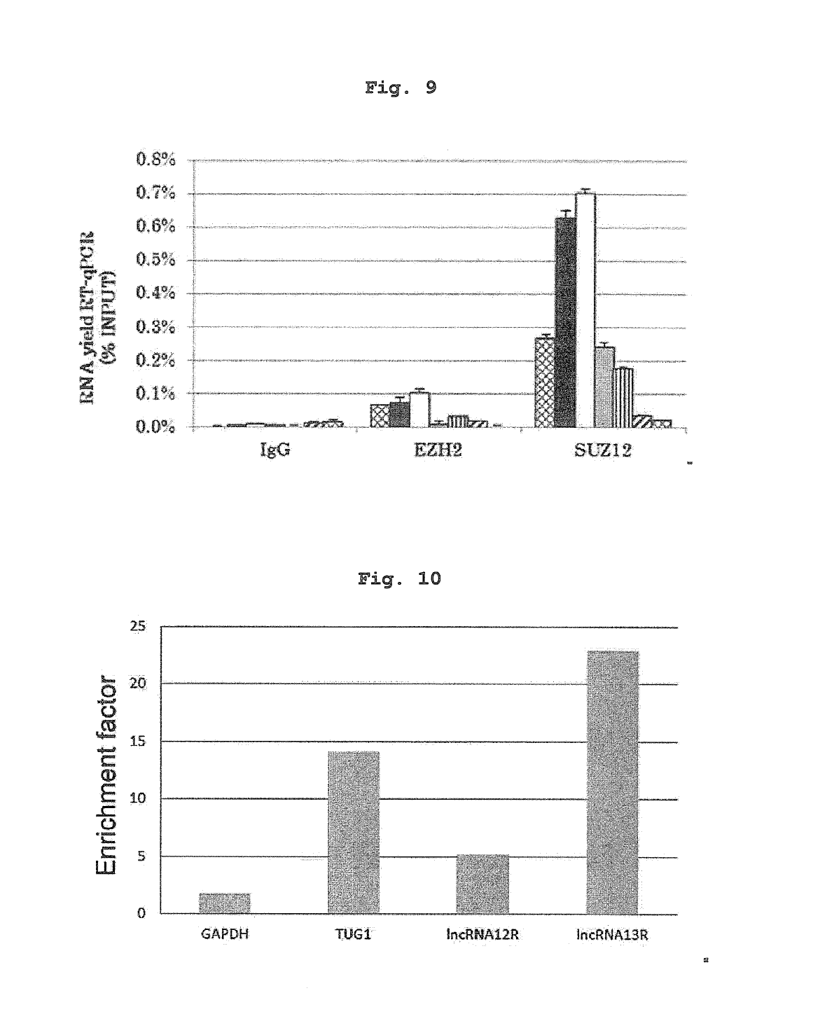

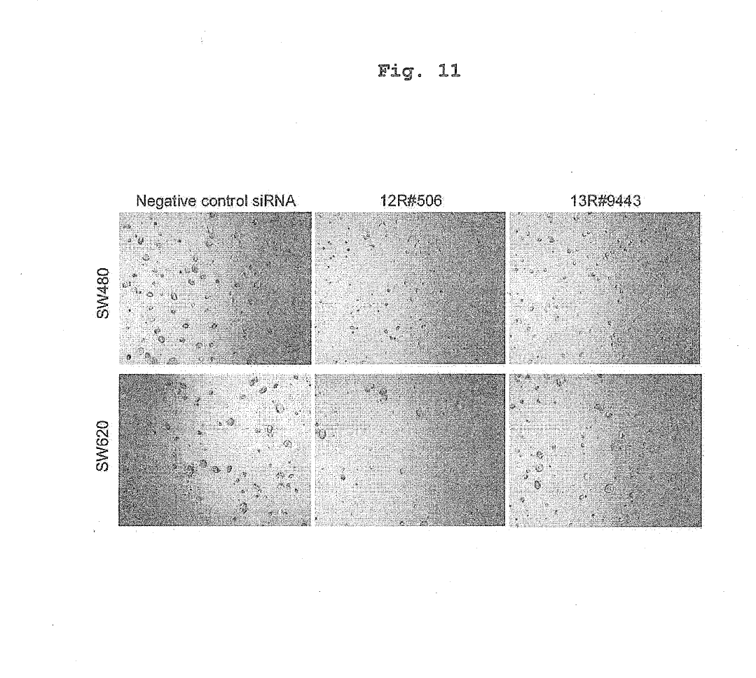

Sequencing library was prepared from the obtained RNA by using TruSeq RNA Sample Preparation Kits (Illumina, Inc.), and sequencing was performed using sequencer Hiseq2000 (Illumina, Inc.). After analysis by TOPHAT and normalization by the number of read mapped on mitochondria, the number of the read mapped in the lncRNA region was counted, and the ratio of the read number with anti-SUZ12 antibody to that with control IgG was calculated, based on which the bindability to PRC2 was evaluated. GAPDH (Glyceraldehyde_{—}3-phosphate_dehydrogenase gene) was used as the negative control and TUG1 (Taurine upregulated gene 1 gene) was used as the positive control. As a result, it was shown that lncRNA13R strongly binds to PRC2 (FIG. 10).

### Example 6

### Suppressive Effect of Colony Formability of Cancer Cells by Suppression of Expression of lncRNA12R and lncRNA13R

siRNAs for lncRNA12R and lncRNA13R (custom synthesized siRNAs, GeneDesign Inc.) were each introduced into colorectal cancer cell line SW480 and colorectal cancer-derived lymph node metastasis cell line SW620 (3×10⁵ cells) to a final concentration of 50 nM by using Lipofectamine 2000 (Life Technologies) and according to the attached protocol. The target sequences of siRNA used are shown in Table 4 (SEQ ID NOs: 42, 45). The control siRNA used was AllStars Negative Control siRNA (QUIAGEN, 1027281). After 24 hr from siRNA introduction, a soft agar medium and cells were mixed, 1.5×10³ cells were seeded again and, 8 days later, formed colonies were photographed using In Cell Analyzer 1000 (GE Healthcare Bio-Sciences Corp.). As a result, the colony formability was suppressed by introducing siRNA 12R#506, 13R#9443 into colorectal cancer cell line SW480, and siRNA 12R#506 into colorectal cancer-derived lymph node metastasis cell line SW620 (FIG. 11).

From the above, a suppressive effect of the suppression of expression of lncRNA12R and lncRNA13R on the colony formability of cancer cells was confirmed.

### Example 7

### Suppressive Effect on Migratory Ability of Cancer Cells by Suppression of Expression of lncRNA12R

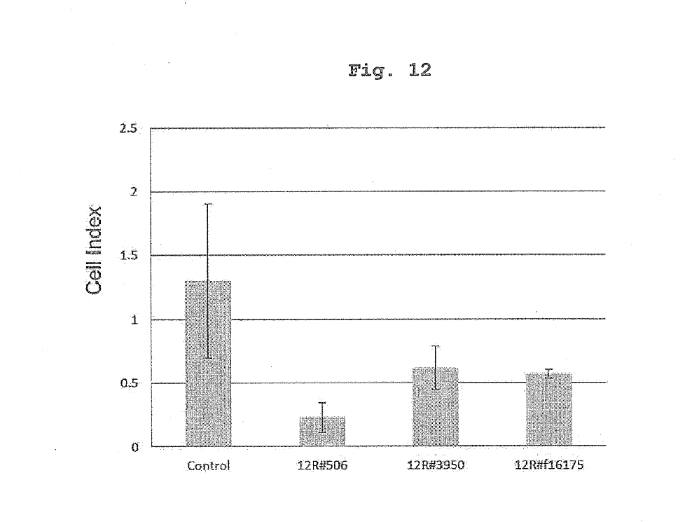

siRNA for lncRNA12R (custom synthesized siRNA, GeneDesign Inc.) was introduced into colorectal cancer cell line SW480 by a method similar to that in Example 6. The target sequences of siRNA used are shown in Table 4 (SEQ ID NOs: 42-44). The control siRNA used was AllStars Negative Control siRNA (QUIAGEN, 1027281). After 24 hr from siRNA introduction, the cells were suspended in a serum-free medium, and 4×10⁴ cells were seeded on an upper well of xCELLigence Real Time Cell Analyzer DP (Roche Diagnostics) CIM plate 16. The wells were connected by filling the lower wells with a medium containing serum, and the migratory ability was evaluated using the serum as an attractant. As a result, the migratory ability of the colorectal cancer cell line was suppressed by decreasing the expression of lncRNA12R by siRNA introduction (FIG. 12).

From the above, a suppressive effect of the suppression of expression of lncRNA12R on the migratory ability of cancer cells was confirmed.

### INDUSTRIAL APPLICABILITY

According to the present invention, novel lncRNA induced by β-catenin can be provided. By screening for a nucleic acid or substance that suppresses expression of the lncRNA, cancer can be diagnosed or treated.

This application is based on a U.S. provisional patent application No. 61/727,185 filed on Nov. 16, 2012, the contents of which are incorporated in full herein by reference.

1. An lncRNA consisting of a nucleotide sequence having not less than 80% identity with the nucleotide sequence shown in any of SEQ ID NOs: 1-15 and SEQ ID NOs: 38-41.

2. An lncRNA that hybridizes to a complementary strand of a nucleic acid consisting of the nucleotide sequence shown in any of SEQ ID NOs: 1-15 and SEQ ID NOs: 38-41 under stringent conditions.

3. An lncRNA consisting of the nucleotide sequence shown in any of SEQ ID NOs: 1-15 and SEQ ID NOs: 38-41.

4. A nucleic acid consisting of a nucleotide sequence complementary to the lncRNA of claim 1.

5. A double-stranded nucleic acid consisting of the lncRNA of claim 1, and a nucleic acid consisting of a nucleotide sequence complementary to the nucleotide sequence of the lncRNA.

6. A nucleic acid that suppresses expression of the lncRNA of claim 1.

7. The nucleic acid according to claim 6, which is selected from siRNA, antisense nucleic acid, shRNA or miRNA.

8. The nucleic acid according to claim 6, which is an siRNA targeting the nucleotide sequence shown in any of SEQ ID NOs: 42-50.

9. A vector expressing the lncRNA of claim 1.

10. A cell introduced with the of lncRNA of claim 1.

11. A cell introduced with the vector of claim 9.

12. A cell growth promoter or growth inhibitor comprising the lncRNA of claim 1 as an active ingredient.

13. A diagnostic drug or a therapeutic drug for a disease caused by abnormality in cell growth, which comprises the lncRNA of claim 1 as an active ingredient.

14. The diagnostic drug or therapeutic drug according to claim 13, wherein the disease is selected from gastrointestinal cancer, liver cancer, kidney cancer, lung cancer, skin cancer, breast cancer, uterine cancer, prostate cancer, urinary bladder cancer, or head and neck cancer.

15. A method of detecting expression of lncRNA, comprising using the lncRNA of claim 1.

16. A method of detecting a mutation of lncRNA, comprising using the lncRNA of claim 1.

17. A method of suppressing expression of lncRNA, comprising using the nucleic acid of claim 4.

18. A method of screening for a substance that suppresses expression or function of lncRNA, comprising using the lncRNA of claim 1.
